SEQUENCE LISTING (8 sequences)

SEQ ID NO: 1 - protein, 38 aa, synthetic construct
CLLGDFFRKSKEKIGKEFKRIVQRIKDFLRNLVPRTES

SEQ ID NO: 2 - protein, 13 aa, synthetic construct
CRWWRWWRRWWRR

SEQ ID NO: 3 - protein, 19 aa, synthetic construct
CWRRWWRRWWRWWRRWWRR

SEQ ID NO: 4 - protein, 35 aa, synthetic construct
CRWWRWWRRWWRKWWRWWRRWWRKWWRWWRRWWRR

SEQ ID NO: 5 - protein, 6 aa, synthetic construct
CLLGDF

SEQ ID NO: 6 - protein, 18 aa, synthetic construct
CLLGDFRWWRWWRRWWRR

SEQ ID NO: 7 - protein, 24 aa, synthetic construct
CLLGDFWRRWWRRWWRWWRRWWRR

SEQ ID NO: 8 - protein, 40 aa, synthetic construct
CLLGDFRWWRWWRRWWRKWWRWWRRWWRKWWRWWRRWWRR

(19) United States
(12) Patent Application Publication
(10) Pub. No.: US 2025/0276065 A1
(43) Pub. Date: Sep. 4, 2025

(54) Electro Magnetic Transducer Network in the Enhancement of Anti-infective Strategies

(71) Applicant: THE REGENTS OF THE UNIVERSITY OF CALIFORNIA, Oakland, CA (US)

(72) Inventors: Fabrizio Billi, Los Angeles, CA (US); Beth Lazzazera, Oakland, CA (US)

(21) Appl. No.: 18/858,988

(22) PCT Filed: Apr. 25, 2023
(86) PCT No.: PCT/US23/66182

§ 371 Date: Oct. 22, 2024

Related U.S. Application Data
(60) Provisional application No. 63/334,291, filed on Apr. 25, 2022.

(51) Int. Cl. A61K 41/00; A61K 31/43; A61K 31/431; A61K 31/545; A61K 31/7036; A61K 33/00; A61K 33/242; A61K 33/26; A61K 33/30; A61K 33/38; A61K 38/14; A61K 47/64; A61P 31/04

(52) U.S. Cl. CPC A61K 41/0052; A61K 31/43; A61K 31/431; A61K 31/545; A61K 31/7036; A61K 33/00; A61K 33/242; A61K 33/26; A61K 33/30; A61K 33/38; A61K 38/14; A61K 47/64; A61P 31/04

(57) ABSTRACT

The present invention provides a composition and a method of treating and preventing biofilm formation and/or eradicating established biofilms in persistent infections, such as in chronic wounds, surgical site infections (SSIs) across multiple specialties, including orthopedics and neurosurgery. In some embodiments, the present invention comprises introducing a composition into a biofilm, the composition comprising nanoparticles that can couple with electromagnetic radiation. In some embodiments, the composition of the present invention may further comprise an antibiotic to help with biofilm eradication/prevention.

### CROSS-REFERENCE TO RELATED APPLICATIONS

This application claims priority to, and benefit of, U.S. Provisional Application No. 63/334,291, filed on Apr. 25, 2022, the disclosure of which is incorporated herein by reference in its entirety.

### STATEMENT REGARDING FEDERALLY SPONSORED RESEARCH OR DEVELOPMENT

This invention was made with government support under Grant Number W81XWH-18-1-0123, awarded by the Department of Defense. The government has certain rights in the invention.

### INCORPORATION BY REFERENCE OF A SEQUENCE LISTING

The present application hereby incorporates by reference the entire contents of the XML filed named “206030-0247-00WO_SequenceListing.XXML” in XML format, which was created on Apr. 25, 2023, and is 8,192 bytes in size.

### BACKGROUND OF THE INVENTION

Biofilms are consortia of micro-organisms that form on various surfaces and are implicated in persistent infections, such as in chronic wounds, surgical site infections (SSIs) across multiple specialties, including orthopedics and neurosurgery. One of the typical properties of biofilm cells is their decreased sensitivity to antimicrobial agents (AMA) as compared to non-adherent cells. One of the important reasons for antimicrobial resistance of biofilms is hindered penetration of AMA through biofilms. If the rate of antibiotic penetration through a biofilm is decreased, the organisms may initially be exposed to a low concentration of the antibiotic and may have time to mount a defensive response. Hindered diffusion is caused by the fact that cells are often packed together in dense clusters of tens to hundreds of micrometers in size, due to which AMA cannot easily reach deep cell layers. Furthermore, cells are surrounded by a biofilm matrix composed of exopolysaccharides (EPS), proteins and extracellular DNA (eDNA). The biofilm matrix can hinder AMA diffusion due to physicochemical interactions of AMA with matrix constituents. A potential way to improve AMA diffusion to cells in a biofilm is by interfering with the biofilm structure so that cells are released from their protective environment.

To interfere with the biofilm structure, the most common approach is to treat the biofilms with pharmacological compounds. For instance, this can be achieved by interfering with quorum sensing or by degrading the EPS matrix (e.g., using the glycoside hydrolase dispersin B) or eDNA (e.g., using deoxyribonuclease I). However, matrix polymers are often stabilized (e.g., stabilization of eDNA with the protein IHF in Burkholderia cenocepacia biofilms) and cannot be easily degraded. Also, as the matrix composition may display quantitative and qualitative variations between different strains, matrix-degrading compounds cannot be broadly applied due to their high specificity. This is corroborated by the fact that in medical settings numerous microbial species may grow within the same biofilm, further increasing the biochemical heterogeneity of the matrix.

Thus, there is a need in the art to develop an anti-infective therapeutic in the setting of complex infections. The present invention meets this need.

### SUMMARY OF THE INVENTION

In one aspect, the present invention provides a method for disrupting a biofilm comprising: a) contacting a biofilm with a nanoparticle, wherein the nanoparticle comprises: one or more metals; and one or more targeting ligands on the surface of the nanoparticle; b) interacting the one or more targeting ligands with one or more selected from the group consisting of: the biofilm matrix, a cell membrane of a bacterium in the biofilm, and a cell wall of a bacterium in the biofilm; c) activating the nanoparticles by exposing them to an electromagnetic field; and d) disrupting the biofilm via the activated nanoparticles. In some embodiments, the one or more metals are one or more selected from the group consisting of: gold, silver, titanium, iron, zinc, and oxides thereof. In some embodiments, the one or more targeting ligand is selected from the group consisting of a lectin, a quorum sensing (QS) signaling molecule, and combinations thereof. In some embodiments, the one or more targeting ligands are one or more selected from the group consisting of: concanavalin-A, LL-37 (SEQ ID NO:1), WR12 (SEQ ID NO:2), WR18 (SEQ ID NO:3), WR34 (SEQ ID NO:4), LL-37-Linker-WR12 (SEQ ID NO:6), LL-37-Linker-WR18 (SEQ ID NO:7), and LL-Linker-WR34 (SEQ ID NO:8).

In some embodiments, the biofilm is located within a subject. In some embodiments, the method further comprises administering an effective dose of at least one first therapeutic agent to the subject. In some embodiments, the at least one first therapeutic agent is one or more selected from the group consisting of an antibiotic, an antiseptic, and combinations thereof. In some embodiments, the antibiotic is selected from the group consisting of: oxacillin, vancomycin, cephalexin, gentamicin, augmentin, amikacin, and combinations thereof. In some embodiments, at least one first therapeutic agent is administered to the subject by at least one method selected from the group consisting of: intra-respiratory, nasal, topical, oral, intravenous, intraperitoneal, intramuscular, transmucosal, buccal, rectal, vaginal, sublingual topically, and combinations thereof.

In some embodiments, the at least one nanoparticle comprises at least one second therapeutic agent. In some embodiments, the at least one second therapeutic agent is selected from the group consisting of: anti-viral agents, anti-bacterial agents, anti-biofilm agents, chemotherapeutic agents, anti-inflammatory agents, antiseptics, anesthetics, analgesics, pharmaceutical agents, small molecules, peptides, nucleic acids, and combinations thereof.

In some embodiments, the composition is administered to the subject by at least one method selected from the group consisting of: intra-respiratory, nasal, topical, oral, intravenous, intraperitoneal, intramuscular, transmucosal, buccal, rectal, vaginal, sublingual topically, and combinations thereof.

In some embodiments, the electromagnetic field is one selected from the group consisting of: a static electromagnetic field, a time-varying electromagnetic field, and a pulsed electromagnetic field, and a combinations thereof. In some embodiments, the pulsed electromagnetic filed has a pulse length ranging between 0.01 seconds to 24 hours. In some embodiments, the electromagnetic field has a frequency ranging between 1 to 800 kHz. In some embodiments, the electromagnetic field has an amplitude ranging between 0.5 to 2 mT. In some embodiments, electromagnetic field has a waveform selected from the group consisting of: a square waveform, a triangular waveform, and combinations thereof. In some embodiments, the electromagnetic field is a high frequency electromagnetic field. In some embodiments, the electromagnetic field is a low frequency electromagnetic field.

In some embodiments, the administration of the at least one first therapeutic agent occurs at least partially simultaneously with at least one of: administration of the nanoparticles and positioning in electromagnetic field. In some embodiments, the administration of the at least one first therapeutic agent occurs before administration of the nanoparticles and positioning in electromagnetic field. In some embodiments, the administration of the at least one first therapeutic agent occurs after administration of the nanoparticles and positioning in electromagnetic field.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A/and FIG. 1B, depicts representative graphs demonstrating that PEMF inhibits S. epidermidis biofilm formation and planktonic cell growth. FIG. 1A depicts a representative graph demonstrating the mean absorbance obtained from CV assay for total biofilm biomass. FIG. 1B depicts a representative graph demonstrating the mean colony forming units per milliliter (CFU/ml) of planktonic S. epidermidis cells. Samples were treated for the indicated length of PEMF exposure (8 hour PEMF exposure group was exposed to PEMF for four hours, removed from exposure for the subsequent 4 hours, then re-exposed for four additional hours). For FIG. 1A and FIG. 1B, data are presented as mean±standard deviation; n=36; *, p<0.05.

FIG. 2 depicts a representative graph demonstrating that PEMF potentiates the disruptive effect of antibiotics on S. epidermidis biofilm formation as observed by CV assay for total biofilm biomass. Samples were treated for the indicated length of PEMF exposure (8 hour PEMF exposure group was exposed to PEMF for four hours, removed from exposure for the subsequent 4 hours, then re-exposed for four additional hours). In the indicated samples, oxacillin or vancomycin was added to the culture media. Data are presented as mean±standard deviation; n=9, *, p<0.001 relative to no-exposure control; **, p<0.001 relative to theoretical additive effect calculated from separately administered PEMF and antibiotic treatment.

FIG. 3 depicts a representative graph demonstrating AlamarBlue cell viability quantification of pre-formed S. epidermidis biofilms when treated with PEMF and PEMF combined with oxacillin or vancomycin at varying durations of PEMF exposure (8 hour PEMF exposure group was exposed to PEMF for four hours, removed from exposure for the subsequent 4 hours, then re-exposed for four additional hours). Data are presented as mean±standard deviation; n=9, *, p<0.001 relative to no-exposure control; **, p<0.001 relative to theoretical additive effect calculated from separately administered PEMF and antibiotic treatment.

FIG. 4, comprising FIG. 4A through FIG. 4F, depicts representative scanning electron microscopy (SEM) images demonstrating that PEMF reduces the amount of surface area covered by S. epidermis biofilm. Shown are SEM image of S. epidermidis grown on a cell-culture treated, polystyrene, 24-well plate. FIG. 4A, FIG. 4B, and FIG. 4C are of S. epidermidis ATCC 14990, and FIG. 4D, FIG. 4E, and FIG. 4F are of S. epidermidis ATCC 35984. FIG. 4A and FIG. 4D show the control, which was S. epidermidis that was neither exposed to PEMF nor vancomycin. FIG. 4B and FIG. 4E show S. epidermidis after 24 hours of PEMF exposure. FIG. 4C and FIG. 4F show S. epidermidis after 24 hours of PEMF exposure and simultaneous vancomycin treatment.

FIG. 5 depicts a schematic representation of the disruption of orthopedic biofilm-based infections by a combination of functionalized iron-core (magnetic) nanoparticles (briefly functionalized nanoparticles, fNPs) and pulsed electromagnetic field (PEMF).

FIG. 6 depicts representative effects of fNPs and PEMF on biofilm eradication.

FIG. 7 depicts representative effects of non-functionalized nanoparticles (NPs) and PEMF on biofilm eradication.

FIG. 8 depicts a schematic representation of an external, non-contact electromagnetic stimulating device coupled with wound dressings to deliver targeted functionalized nanoparticles for sustained antimicrobial capability and promote wound healing.

FIG. 9 depicts representative effects of various treatments on biofilm eradication.

FIG. 10 depicts a schematic representation of a workflow of an ex vivo burn wound model.

FIG. 11 depicts a schematic representation of a workflow of a pig skin wound model.

FIG. 12 depicts a representative diagram of a functionalized magnetic nano particle (fMNP) based wound dressing.

FIG. 13 depicts a schematic representation of an electromagnetic acoustic transducer (EMAT). Alternating current feeds the induction coil, causing electromagnetic oscillations, which in turn induce eddy currents on the surface of the metal alloy. The induced eddy current interferes with the permanent magnetic field, creating ultrasonic waves directly on the surface of the test object.

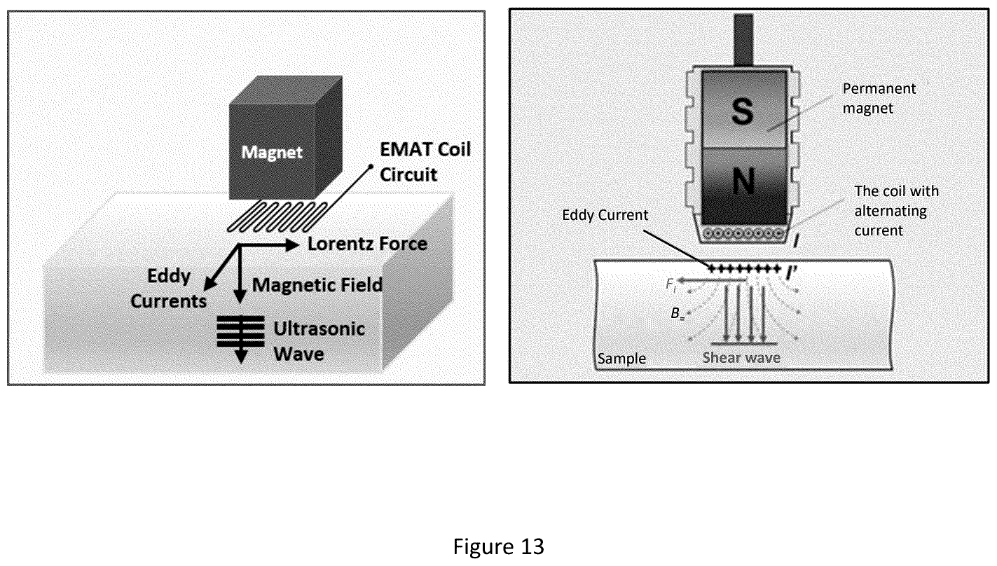

FIG. 14, comprising FIG. 14 A and FIG. 14B, depicts representative influence of the lift-off distance h on Lorentz force. FIG. 14A depicts the simulated effect using a computer model. FIG. 14B depicts representative in vitro results.

FIG. 15 depicts images of a representative apparatus for exposure of bacteria cultures to EMF. The wires form the coil that delivers the EMF at the tray.

FIG. 16 depicts images a representative coil-embedded mat. A coil that is inserted in the mat is placed over the mat (left) for reference; the mats are placed in the incubator (middle). A schematic representation of a well plate placed on the mat for testing is also depicted (right).

FIG. 17 depicts a schematic representation of soft tissue phantom interposed between the EMF mat and the well plates.

FIG. 18 depicts representative results obtained for PEMF inhibiting S. epidermidis biofilm formation on cell-culture treated plastic 6-well plates. Significant inhibition was seen at all PEMF exposure times measured.

FIG. 19 depicts representative results obtained for PEMF inhibiting S. epidermidis planktonic cell growth on cell-culture treated 6-well plates. Significant inhibition was seen at 12 and 24 hours of exposure.

FIG. 20 depicts representative results obtained for eradication of a pre-formed S. epidermidis biofilm by PEMF and PEMF+oxacillin on cell-culture treated plastic 6-well plates. Significant eradication was seen at all time points, and significant empirical synergism was seen between PEMF, and oxacillin as compared to their theoretical additive effect.

FIG. 21 depicts representative alamarBlue Fluorescence data obtained for S. epidermidis biofilm eradication by PEMF on metal alloy discs.

FIG. 22 depicts representative AlamarBlue fluorescence data obtained for S. epidermidis biofilm eradication by PEMF, PEMF+vancomycin, PEMF+concanavalin A (Con-A) fNPs, and PEMF+vancomycin+Con-A fNPs on cell-culture treated plastic 24-well plates.

FIG. 23 depicts a representative image of 3D osteoblast/osteocyte ring cultures visualized by fluorescence.

FIG. 24 depicts a representative image of dendritic extension after matrix demineralization in a 3D bone model.

FIG. 25 depicts representative results of quasi-real-time mineralization monitoring in a 3D bone model via intravital ARS fluorescence of human-adipose-derived stem cells (hADSCs) driven by BMP9.

FIG. 26 depicts representative images of elemental analysis of bone mineralization in a 3D bone ring model via EDX/scanning electron microscopy examining calcium (top) and phosphorus (bottom).

FIG. 27 depicts representative effects of bacteria on mineral content of a 3D bone ring model with hADSCs. Bacteria were added to the model on day 21, after which a drastic reduction in ARS fluorescence was observed, suggesting bacteria are responsible for demineralization of the bone ring.

FIG. 28 depicts a schematic representation of low (LD) and high density (HD) nanoparticles, as well as Very High Focal Density (VHFD) nanoparticles resulting from conjugation of dendrons as depicted.

FIG. 29 depicts a schematic representation of a representative pulsed electromagnetic field (PEMF) device for animal experiments.

FIG. 30 depicts a schematic representation of a timeline for a pilot animal study on the efficacy of targeted magnetic nanoparticles (fMNPs) and a pulsed electromagnetic field (PEMF) on biofilm degradation.

FIG. 31 depicts a schematic representation of a timeline for an in-depth animal study on the efficacy of targeted magnetic nanoparticles (fMNPs) and a pulsed electromagnetic field (PEMF) as well as the impact of antibiotics (ABX) on biofilm degradation.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of compositions and methods of treating/disrupting biofilms. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates generally to compositions and methods to destroy, disrupt, or inhibit biofilm formation. In some embodiments, the invention is used to treat and/or prevent biofilms and infectious diseases caused by biofilms. Examples of where the invention is useful for treating and/or preventing biofilms include, but are not limited to, surgical site infections, burn infections, orthopedic implant infections, etc. In some embodiments, the invention is used to target bacteria in planktonic state. In some embodiments, the invention is used to treat and/or prevent infectious diseases caused by bacteria in planktonic state. In some embodiments, the invention may be used to treat orthopedic surgical or trauma sites by allowing penetration of therapeutic agents into osteocyte-lacuno canaliculi networks and killing hiding bacteria that have adopted a dormant small colony variant (SCV) phenotype. In some embodiments, the invention may be used to treat and/or prevent osteomyelitis. In some embodiments, the invention may be used to disrupt or kill viruses. In some embodiments, the invention may be used to treat cancer.

The present invention is not limited to the treatment or prevention of biofilms in medical settings, but also encompasses the treatment or prevention of biofilms in environmental, commercial, and industrial settings.

In some embodiments, the present invention may be used in veterinary field. In some embodiments, the present invention may be used in any animal including but not limited to horses (more specifically racehorses), dogs, cats, etc.

### Nanoparticles

In some embodiments, the present invention provides nanoparticles that inhibit the formation of biofilms. In some embodiments, the nanoparticles inhibit further accumulation of biofilm. In some embodiments, the nanoparticles disrupt existing biofilms. In some embodiments, the nanoparticles eliminate existing biofilms. In some embodiments, the nanoparticles promote the disruption or disassembly of existing biofilms. In some embodiments, promoting the disruption or disassembly of existing biofilms allows for easier mechanical biofilm disruption. In some embodiments, the composition weakens an existing biofilm, allowing for easier mechanical biofilm disruption.

In some embodiments, the nanoparticle is capable of binding to a biofilm and/or to sites at risk for biofilm formation and accumulation. In some embodiments, the nanoparticles are capable of binding to a bacterial component of a biofilm.

In various embodiments, the nanoparticles can react with electromagnetic radiation. In some embodiments, the nanoparticles are magnetic or electromagnetic nanoparticles (MNPs). In some embodiments, the nanoparticles are metal nanoparticles, nanoparticles with core-shell structures, or electro-ceramic nanocomposites. Examples of suitable nanoparticles include, but are not limited to, gold nanoparticles (AuNPs), silver nanoparticles (AgNPs), titanium nanoparticles (TiNPs), iron nanoparticles (FeNPs), iron oxide nanoparticles (FeONPs), zinc oxide nanoparticles (ZnONPs), graphene oxide based nanoparticles with a metal shell or core, carbon nanotubes with a metal shell or core, and carbon dots with a metal shell or core. Different surface charges may be applied to the nanoparticle. For example, the structure may be anionic, neutral, or cationic.

In some embodiments, the nanoparticles are FeONPs. Iron oxides are capable of generating reactive oxygen species by reacting with hydrogen peroxide via the Fenton reaction, which in turn may contribute to antimicrobial activity. In some embodiments, the nanoparticles are ZnONPs.

In some embodiments, the nanoparticles are functionalized. In some embodiments, the particles have a functionalized surface. Nanoparticles may have one or more surface functionalizations. In some embodiments, the nanoparticles are functionalized for one or more purpose. Examples of purposes of functionalization include, but not limited to, improving colloidal stability, obtaining a certain surface charge, coupling of antimicrobial agents, and targeting. Examples of surface functionalizations include, but are not limited, to polyethylene amine (PEI), polyethylene glycol, polysaccharides, lipids, lectins, antibodies, peptides, amino acids, and aptamers.

In some embodiments, the nanoparticles are functionalized by any means known in the art. Examples of means by which nanoparticles can be functionalized include, but are not limited to, N-hydroxy succinimide coupling, maleimide coupling, alkyne-azide coupling, Diels-Alder coupling, thiol-ene coupling, and thiol-Michael coupling.

In some embodiments, nanoparticles are coated with a self-generated extracellular matrix composed of exopolysaccharides, proteins, and DNA.

In some embodiments, the nanoparticles are functionalized with one or more targeting molecules that aid in selective binding of the nanoparticle to bacteria or biofilms. In some embodiments, the nanoparticles targeting molecules are one or more proteins or peptides. In some embodiments, the targeting molecule is a lectin that binds sugars. In some embodiments, the lectin is one found in the exopolysaccharides of a biofilm matrix. In some embodiments, the lectin is Wheat Germ Agglutinin (WGA). In some embodiments, targeting molecule is a quorum sensing molecules. In some embodiments, the quorum-sensing molecule is the autoinducing peptide (AIP) of S. aureus. In some embodiments, the targeting molecule is an antimicrobial peptide. In some embodiments, the antimicrobial peptide facilitates targeting of the nanoparticle as well as killing of bacteria and/or destruction of biofilms. In some embodiments, the antimicrobial peptide disrupts the membrane structure and generates transmembrane peptide-dependent channels that depolarize the membrane. In some embodiments, the antimicrobial peptide is one or more selected from the group consisting of concanavalin-A (Con-A), LL-37 (SEQ ID NO:1), WR12 (SEQ ID NO:2), WR18 (SEQ ID NO:3), WR34 (SEQ ID NO:4), WR12 with an LL-37 linker (SEQ ID NO:5 and SEQ ID NO:2), WR18 with an LL-37 linker (SEQ ID NO:5 and SEQ ID NO:3), and WR34 with an LL-37 linker (SEQ ID NO:5 and SEQ ID NO:4).

In some embodiments, the nanoparticles are labeled with a fluorescent dye to allow visualization after administration. In some embodiments, the nanoparticles are labeled with at least one Alexa Fluor fluorescent dye.

In some embodiments, the nanoparticles further comprise at least one therapeutic agent. In some embodiments, the nanoparticles comprise a combination of two or more therapeutic agents.

The present invention is not limited to any particular therapeutic agent, but rather encompasses any suitable therapeutic agent that can be embedded within or coupled to the nanoparticle. Exemplary therapeutic agents include, but are not limited to, anti-viral agents, anti-bacterial agents, anti-biofilm agents, chemotherapeutic agents, anti-inflammatory agents, antiseptics, anesthetics, analgesics, pharmaceutical agents, small molecules, peptides, nucleic acids, and the like. In some embodiments, the therapeutic agent comprises an anti-biofilm agent, including but not limited to apigenin and derivatives thereof; myricetin and derivatives thereof, flavonoids including flavones, flavonols, dihydroflavonols, flavonones, and derivatives thereof, farnesol and derivatives thereof; terpenoids including terpenes, terpinols, diterpenic acids, diterpenes, triterpenes, and derivatives thereof, biofilm degrading enzymes including mutanase, dextranase, and amyloglucosidade-glucose oxidase; and EPS-synthesizing enzyme inhibitors including Rose Bengal, Perborate, meta-periodate, sorbitol, xylitol, 1-deoxynojirimycin, flavonoids, polyphenols, proanthocyanidins, tannins, and coumarins.

In another embodiment, the at least one therapeutic agent comprises an antibacterial agent, including but not limited to chlorhexidine and derivatives thereof, members of the bisbiguanide class of inhibitors, povidone iodine, hydrogen peroxide, a hydrogen peroxide-donor, a peroxide donor, a superoxide donor, doxycycline, minocycline, clindamycin, doxycycline, metronidazole, essential oil extracts, menthol, thymol, eucalyptol, methyl salicylate, metal salts, zinc salts, copper salts, stannous salts, phenols, triclosan, quaternary ammonium compounds, cetylpyridinium chloride, surfactants, sodium lauryl sulphate, delmopinol, and natural products (phenols, phenolic acids, quinones, alkaloids, lectins, peptides, polypeptides, indole derivatives, flustramine derivatives, carolacton, halogenated furanones, oroidin analogues, agelasine, ageloxime D).

In some embodiments, the nanoparticles retain the at least one therapeutic agent until triggered to release the at least one therapeutic agent, thereby providing targeted release of the therapeutic agent only when and where it is most needed. In some embodiments, the targeted release of the therapeutic agent minimizes off-target effects. In some embodiments, the targeted release of the therapeutic agent creates a microenvironment of high concentration of the therapeutic agent, thereby reducing the required dose. Examples of release triggers include, but are not limited to, temperature, pH, biomolecule recognition, and the like.

In some embodiments, the composition may comprise a plurality of different nanoparticles, wherein each of the different nanoparticles comprise a different therapeutic agent. For example, In some embodiments, the composition comprises a first nanoparticle, comprising one or more anti-biofilm agents, and a second nanoparticle, comprising one or more antibiotics. In another embodiment, the composition comprises a first nanoparticle, comprising an anti-biofilm agent, a second nanoparticle, comprising a broad-spectrum antibiotic, and a third nanoparticle, comprising an antiseptic. In some embodiments, each of the different nanoparticles is configured for different drug delivery characteristics, thereby allowing different therapeutic agents to be delivered at different times, as necessitated by the particular disorder or treatment.

In some embodiments, a nanoparticle provided herein is self-assembled. In certain embodiments, the nanoparticle is self-assembled or is capable of being self-assembled in an aqueous medium. In some embodiments, the nanoparticle is self-assembled or is capable of being self-assembled in human serum. In some embodiments, the nanoparticle is self-assembled or is capable of being self-assembled in a membrane or membrane-like environment to form amphipathic, alpha-helical antiparallel dimers and tetramers.

In some embodiments, a nanoparticle provided herein self-assembles at any suitable concentration. In certain embodiments, a nanoparticle provided herein self-assembles (e.g., has a critical assembly concentration (CAC), or the minimum concentration at which a nanoparticle forms) of about 2 μg/mL, about 5 μg/mL, about 8 μg/mL, about 10 μg/mL, about 20 μg/mL, about 25 μg/mL, about 30 μg/mL, about 40 μg/mL, about 50 μg/mL, about 60 μg/mL, about 70 μg/mL, about 80 μg/mL, about 90 μg/mL, about 100 μg/mL, or greater. In certain embodiments, a nanoparticle provided herein may self assembles at any suitable concentration below 2 μg/mL.

In some embodiments, the nanoparticles provided herein is of any suitable size. In some embodiments, the size of the nanoparticles is adjusted to meet specific needs by adjusting the degree of polymerization of the core sections, shell sections, additional sections, or a combination thereof. In some embodiments, the nanoparticle has an average diameter of between about 0.1 nm and about 10 m. In some embodiments, the nanoparticle has an average diameter of between about 1 nm and about 1 μm. In some embodiments, the nanoparticle has an average diameter of between about 5 nm and about 500 nm. In some embodiments, the nanoparticle has an average diameter of between about 10 nm and about 250 nm. In some embodiments, the nanoparticle has an average diameter of between about 25 nm and about 250 nm. In some embodiments, the nanoparticle has an average diameter of between about 50 nm and about 250 nm. In some embodiments, the nanoparticle provided herein has an average diameter of about 60 nm to 200 nm. In some embodiments, the nanoparticle has an average diameter of about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 300 nm, about 350 nm, about 400 nm, about 450 nm, about 500 nm, about 600 nm, about 700 nm, about 800 nm, about 900 nm, or about 1,000 nm.

### Compositions

In one aspect, the present disclosure provides compositions that comprise one or more nanoparticles of the present invention. In some embodiments, the composition comprises one or more nanoparticles that inhibit the formation of, inhibit accumulation of, disrupt, or eliminate biofilms. Thus, in some embodiments, the composition targets biofilms or locations where biofilms are prone to form.

In some embodiments, the composition comprises at least two different nanoparticles. In some embodiments, the at least two different nanoparticles are two or more different kinds of nanoparticles selected from the group consisting of gold nanoparticles (AuNPs), silver nanoparticles (AgNPs), titanium nanoparticles (TiNPs), iron nanoparticles (FeNPs), iron oxide nanoparticles (FeONPs), zinc oxide nanoparticles (ZnONPs), graphene oxide based nanoparticles with a metal shell or core, carbon nanotubes with a metal shell or core, and carbon dots with a metal shell or core. In some embodiments, the at least two different nanoparticles comprise different targeting molecules. In some embodiments, the at least two different targeting molecules are selected from the group consisting concanavalin-A (Con-A), LL-37 (SEQ ID NO:1), WR12 (SEQ ID NO:2), WR18 (SEQ ID NO:3), WR34 (SEQ ID NO:4), WR12 with an LL-37 linker (SEQ ID NO:5 and SEQ ID NO:2), WR18 with an LL-37 linker (SEQ ID NO:5 and SEQ ID NO:3), and WR34 with an LL-37 linker (SEQ ID NO:5 and SEQ ID NO:4).In some embodiments, at least one of the at least two different nanoparticles is fluorescently labeled. In some embodiments, at least two or more nanoparticles are fluorescently labeled. In some embodiments, all nanoparticles are fluorescently labeled. In some embodiments the at least two different nanoparticles comprise different therapeutic agents. In some embodiments, the at least two different nanoparticles comprise therapeutic agents selected from the group consisting of anti-viral agents, anti-bacterial agents, anti-biofilm agents, chemotherapeutic agents, anti-inflammatory agents, antiseptics, anesthetics, analgesics, pharmaceutical agents, small molecules, peptides, and nucleic acids.

In some embodiments, the composition comprises nanoparticles at any suitable concentration. In some embodiments, the concentration of nanoparticles is less than about 0.0001 μg/mL. In some embodiments, the concentration of nanoparticles is greater than about 1 g/mL. In some embodiments, the concentration of nanoparticles is between about 0.0001 μg/mL and about 1 g/mL. In some embodiments, the concentration of nanoparticles is between about 0.001 μg/mL an about 100 mg/mL. In some embodiments, the concentration of nanoparticles is between about 0.01 μg/mL and about 10 mg/mL. In some embodiments, the concentration of nanoparticles is between about 0.1 μg/mL and about 1 mg/mL. In some embodiments, the concentration of nanoparticles, is between about 1 μg/mL and about 100 μg/mL. In some embodiments, the concentration of nanoparticles is about 0.0001 μg/mL, about 0.001 μg/mL, about 0.01 μg/mL, about 0.1 μg/mL, about 1 μg/mL, about 10 μg/mL, about 100 μg/mL, about 1 mg/mL, about 10 mg/mL, about 100 mg/mL, or about 1 g/mL.

In certain embodiments, the composition of the invention is a solution, foam, paste, gel, candy, gum, dissolvable substrate or film, tablet, lozenge, or the like, which a nanoparticle of the invention can be incorporated into. For example, the composition may be of any form which allows its application onto a surface having a biofilm, or at risk for developing a biofilm.

In some embodiments, the nanoparticles may be embedded in a gel. Examples of suitable gels include, but not limited, agarose gel, polyethylene glycol (PEGylated) fibrin gels, chitosan-based hydrogel, etc. In some embodiments, the nanoparticles are embedded in a 2% agarose hydrogel.

In some embodiments, the composition of the invention is coated upon implantable materials to prevent the formation and/or accumulation of biofilm on the implantable material. In some embodiments, the composition is coated on an implant. Examples of implants include, but are not limited to, orthopedic implants (e.g., plates, screws, artificial joints, etc.), tissue engineered substrates, pacemakers, heart pumps, artificial valves, insulin pumps, breathing tubes, central line catheters, and indwelling catheters.

In some embodiments, the composition comprises nanoparticles in an aqueous solution.

### Methods

In some embodiments, the present invention provides methods of treating and/or preventing infection in a subject. In some embodiments, the method of preventing infection comprises eliminating, reducing the amount of, preventing the formation of, or preventing the accumulation of biofilms. In some embodiments, the method comprises the step of contacting the biofilm, with a composition or nanoparticle of the present invention. In some embodiments, the method of treating and/or preventing infection comprises eliminating or reducing the number of planktonic bacteria that contribute, or may contribute, to an infection.

In some embodiments, the method comprises administering the composition or nanoparticle to a subject. In some embodiments, the administration is systemic. In some embodiments, the administration is local. In some embodiments, the composition or nanoparticle is administered through injection, infusion, perfusion, inhalation, ingestion, transdermal absorption, and absorption through a mucous membrane.

In some embodiments, the method comprises administering a composition or nanoparticle to a location where a biofilm is prone to form. In some embodiments, the location is a location within a subject's body. In some embodiments, the location is a surgical site, burn site, orthopedic implant site, pacemaker implant site, or heart plump implant site. In some embodiments, the composition or nanoparticle is administered through injection, infusion, perfusion, inhalation, ingestion, transdermal absorption, and absorption through a mucous membrane.

In some embodiments, the location is the surface of an implant. Examples of implants include, but are not limited to, orthopedic implants (e.g., plates, screws, artificial joints, etc.), tissue engineered substrates, pacemakers, heart pumps, artificial valves, insulin pumps, breathing tubes, central line catheters, and indwelling catheters. In some embodiments, composition or nanoparticle of the invention is introduced to the implant by any means known in the art. In some embodiments, the composition or nanoparticle is introduced to the implant prior to implantation into a subject by any suitable means known in the art. In some embodiments, the composition or nanoparticle is introduced to the implant by injecting, infusing, perfusing, saturating, or coating the implant with the composition or nanoparticle. In some embodiments, the composition or nanoparticle is introduced to the implant after implantation. In some embodiments, the composition or nanoparticle is introduced to the implant by administration of the composition or nanoparticle to the subject who has received the implant by any suitable means. In some embodiments, the composition or nanoparticle is administered through injection, infusion, perfusion, inhalation, ingestion, transdermal absorption, and absorption through a mucous membrane.

In some embodiments, the disclosure provides methods of eliminating, reducing the amount of, preventing the formation of, or preventing the accumulation of biofilms on an environmental, commercial, or industrial surface. In some embodiments, the method comprises introducing to the surface a composition or nanoparticle of the present disclosure by any suitable means known in the art. In some embodiments, the composition or nanoparticle is introduced to the surface by spraying, painting, soaking, brushing, dusting, adsorbing, or adhering the composition or nanoparticle to the surface.

In some embodiments, biofilm comprises one or more bacteria. In some embodiments, the one or more bacteria is Gram-positive. In some embodiments, the one or more bacteria is Gram-negative. In some embodiments, the biofilm comprises one or more Gram-positive bacteria and one or more Gram-negative bacteria. In some embodiments, the biofilm comprises one or more fungi. In some embodiments, the biofilm comprises one or mor archaea. In some embodiments, the biofilm comprises one or more selected from the group consisting of Staphylococcus epidermidis, Staphylococcus aureus, Streptococcus spp, Pseudomonas aeruginosa, Burkholderia cepacia, Candida spp, Escherichia coli, Streptococcus mutans, Rubus fruticosus, Shewanella oneidensis, Saccharomyces cerevisiae, Bacillus anthracis, Bacillus circulans, Micrococcus luteus, Pseudomonas fluorescens, Salmonella enteritidis, Serratia marcescens, Hordeum vulgare, Mycobacterium tuberculosis, Ervinia carotovora, Streptomyces scabies, Haemophilus spp., Bordetella pertussis, Coxiella burnetii, Klebsiella pneumonia, Mycoplasma pneumonia, Chlamydophila pneumonia, Legionella pneumophila, Moraxella catarrhalis, Yersinia pestis, Heliobacterium pylori, and Alternaria solani. In some embodiments, the biofilm comprises Pseudomonas aeruginosa.

In some embodiments, biofilm may comprise an aerobic and facultative or anaerobic microorganism known to associates with wound infection. In some embodiments, the microbe is one or more selected from the group consisting of coagulase-negative staphylococci, Micrococcus sp., and Staphylococcus aureus.

In some aspects, the present invention is partly based upon the surprising discovery that biofilm formation and cell growth are decreased by the length of exposure to pulsed electromagnetic fields (PEMF) and the viability of preformed biofilms is also be decreased by the length of exposure to PEMF. Accordingly, in some embodiments, the method comprises positioning the subject in an electromagnetic field. In some embodiments, the method comprises positioning a portion of the subject in an electromagnetic field. In some embodiments, the method comprises positioning a targeted site on a subject in an electromagnetic field. In some embodiments, PEMF is configured to affect biofilm formation and strength by interfering with the attractive electrostatic forces between cells and the surface to which they adhere.

In some embodiments, the electromagnetic field is any suitable electromagnetic field. In some embodiments, the electromagnetic field includes one or more selected from the group consisting of a static electromagnetic field, a time-varying electromagnetic field, and a pulsed electromagnetic field. In some embodiments, the pulsed electromagnetic field is a switched electromagnetic field. In some embodiments, the electromagnetic field is switched between about every 0.001 seconds and about every 24 hours. In some embodiments, the electromagnetic field is switched between about every 0.01 seconds and about every 12 hours. In some embodiments, the electromagnetic field is switched between about every 0.1 seconds and about every hour. In some embodiments, the electromagnetic field is switched between about every 1 second and about every 1 minute. In some embodiments, the electromagnetic field is switched about every 0.001 seconds, about every 0.01 seconds, about every 0.1 seconds, about every 0.25 seconds, about every 0.5 seconds, about every 0.75 seconds, about every 1 second, about every 2 seconds, about every 3 seconds, about every 4 seconds, about every 5 seconds, about every 6 seconds, about every 7 seconds, about every 8 seconds, about every 9 seconds, about every 10 seconds, about every 12 seconds, about every 14 seconds, about every 16 seconds, about every 18 seconds, about every 20 seconds, about every 25 seconds, about every 30 seconds, about every 35 seconds, about every 40 seconds, about every 45 seconds, about every 50 seconds, about every 55 seconds, about every 1 minute, about every 2 minutes, about every 3 minutes, about every 4 minutes, about every 5 minutes, about every 6 minutes, about every 7 minutes, about every 8 minutes, about every 9 minutes, about every 10 minutes, about every 12 minutes, about every 14 minutes, about every 16 minutes, about every 18 minutes, about every 20 minutes, about every 25 minutes, about every 30 minutes, about every 35 minutes, about every 40 minutes, about every 45 minutes, about every 50 minutes, about every 55 minutes, about every 1 hour, about every 2 hours, about every 3 hours, about every 4 hours, about every 5 hours, about every 6 hours, about every 8 hours, about every 10 hours, about every 12 hours, about every 14 hours, about every 16 hours, about every 18 hours, or about every 24 hours.

In some embodiments, the electromagnetic field has a frequency of between about 0.01 kHz and about 10,000,000 kHz. In some embodiments, the electromagnetic field has a frequency of between about 0.1 kHz and about 1,000,000 kHz. In some embodiments, the electromagnetic field has a frequency between about 1 kHz and about 100,000 kHz. In some embodiments, the electromagnetic field has a frequency between about 10 kHz and about 100,000 kHz. In some embodiments, the electromagnetic field has a frequency between about 100 kHz and about 100,000 kHz. In some embodiments, the electromagnetic field has a frequency between about 1,000 kHz and about 100,000 kHz. In some embodiments, the electromagnetic field has a frequency between about 10,000 kHz and about 100 kHz. In some embodiments, the electromagnetic field has a frequency between about 1 kHz and about 800 kHz. In some embodiments, the electromagnetic field has a frequency of about 0.01 kHz, about 0.1 kHz, about 1 kHz, about 2 kHz, about 3 kHz, about 4 kHz, about 5 kHz, about 10 kHz, about 20 kHz, about 25 kHz, about 40 kHz, about 50 kHz, about 75 kHz, about 100 kHz, about 125 kHz, about 150 kHz, about 200 kHz, about 250 kHz, about 500 kHz, about 750 kHz, about 1,000 kHz, about 1,500 kHz, about 2,000 kHz, about 2,500 kHz, about 5,000 kHz, about 10,000 kHz, about 20,000 kHz, about 50,000 kHz, about 100,000 kHz, about 250,000 kHz, about 500,000 kHz, about 1,000,000 kHz, about 5,000,000 kHz, or about 10,000,000 kHz. In some embodiments, the electromagnetic field may be a high frequency electromagnetic field. In some embodiments, the electromagnetic field may be a low frequency electromagnetic field.

In some embodiments, the subject or targeted area is subjected to the electromagnetic field for one or more cycles. In some embodiments, each cycle has a suitable duration known to one skilled in the art. In some embodiments, cycles have the same duration. In some embodiments, the cycles have varying durations. In some embodiments, at least one cycle is between about 0.01 seconds and about 24 hours. In some embodiments, the at least one cycle is between about 0.1 seconds and about 12 hours. In some embodiments, the at least one cycle is between about 1 second and about 8 hours. In some embodiments, the at least one cycle is between about 10 seconds and about 4 hours. In some embodiments, the at least one cycle is between about 1 minute and about 1 hour. In some embodiments, the at least one cycle is about 0.01 seconds, about 0.1 seconds, about 1 second, about 2 seconds, about 3 seconds, about 4 seconds, about 5 seconds, about 6 seconds, about 7 seconds, about 8 seconds, about 9 seconds, about 10 seconds, about 12 seconds, about 14 seconds, about 16 seconds, about 18 seconds, about 20 seconds, about 25 seconds, about 30 seconds, about 35 seconds, about 40 seconds, about 45 seconds, about 50 seconds, about 55 seconds, about 1 minute, about 2 minutes, about 3 minutes, about 4 minutes, about 5 minutes, about 6 minutes, about 7 minutes, about 8 minutes, about 9 minutes, about 10 minutes, about 12 minutes, about 14 minutes, about 16 minutes, about 18 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, or about 24 hours.

In some embodiments, the electromagnetic field has an amplitude from between about 0.05 mT and about 20 mT. In some embodiments, the amplitude from between about 0.5 and about 2 mT. In some embodiments, the electromagnetic field has an amplitude of about 0.05 mT, about 0.1 mT, about 0.15 mT, about 0.2 mT, about 0.25 mT, about 0.3 mT, about 0.35 mT, about 0.4 mT, about 0.55 mT, about 0.6 mT, about 0.65 mT, about 0.7 mT, about 0.75 mT, about 0.8 mT, about 0.85 mT, about 0.9 mT, about 0.95 mT, about 1.0 mT, about 1.1 mT, about 1.2 mT, about 1.3 mT, about 1.4 mT, about 1.5 mT, about 1.6 mT, about 1.7 mT, about 1.8 mT, about 1.9 mT, about 2.0 mT, about 2.25 mT, about 2.5 mT, about 2.75 mT, about 3.0 mT, about 3.5 mT, about 4.0 mT, about 4.5 mT, about 5.0 mT, about 6.0 mT, about 7.0 mT, about 8.0 mT, about 9.0 mT, about 10 mT, about 12 mT, about 14 mT, about 16 mT, about 18 mT, or about 20 mT.

In some embodiments, the electromagnetic field has any waveform known of skill to one in the art. In some embodiments, the electromagnetic field has a waveform of one or more selected from the group consisting of a sine waveform, a modified sine waveform, a square waveform, a triangular waveform, a sawtooth waveform, a rectangular waveform, and combinations thereof.

In some aspects, the present invention is partly based upon the surprising discovery that the combination of nanoparticles with PEMF mechanically disrupts biofilms by decreasing the viability of the bacterial cells. Accordingly, in some embodiments, the present invention provides a method of treating and/or preventing an infection. In some embodiments, the infection is associated with one or more selected from the group consisting of a biofilm and planktonic bacteria. In some embodiments, the method comprises the steps of administering a composition or nanoparticle of the present disclosure to a subject and positioning the subject in an electromagnetic field. In some embodiments, the method comprises administering an effective amount of a composition or nanoparticle of the present disclosure to a subject and positioning the subject in an electromagnetic field. In some embodiments, the method comprises administering a therapeutically effective amount of a composition or nanoparticle of the present disclosure to a subject and positioning the subject in an electromagnetic field.

In some embodiments, exposing of the biofilm or bacteria to the electromagnetic field and the administration of the composition or nanoparticle occur independent of one another with respect to time. In some embodiments, exposing of the biofilm to the electromagnetic field and the administration of the composition and nanoparticle occur at least partially simultaneously. In some embodiments, the exposing of the biofilm to the electromagnetic field and the administration of the composition or nanoparticle occur substantially simultaneously. In some embodiments, the exposing of the biofilm to the electromagnetic field occurs after administration of the composition or nanoparticle.

In some embodiments, the exposing of the biofilm to the magnetic field and the administration of the composition or nanoparticle independently occur at any suitable temperature, such as about −100° C. to about 100° C. In some embodiments, the exposing of the biofilm to the magnetic field occurs at a temperature of about −100° C., about −90° C., about −80° C., about −70° C., about −60° C., about −50° C., about −40° C., about −30° C., about −20° C., about −10° C., about 0° C., about 10° C., about 15° C., about 20° C., about 25° C., about 30° C., about 35° C., about 36° C., about 37° C., about 38° C., about 39° C., about 40° C., about 45° C., about 50° C., about 60° C., about 70° C., about 80° C., about 90° C., or about 100° C. In some embodiments, the administration of the composition or nanoparticle occurs at a temperature of about −100° C., about −90° C., about −80° C., about −70° C., about −60° C., about −50° C., about −40° C., about −30° C., about −20° C., about −10° C., about 0° C., about 10° C., about 15° C., about 20° C., about 25° C., about 30° C., about 35° C., about 36° C., about 37° C., about 38° C., about 39° C., about 40° C., about 45° C., about 50° C., about 60° C., about 70° C., about 80° C., about 90° C., or about 100° C. In some embodiments, the exposing of the biofilm to the magnetic field and the administration of the composition occur at 37° C.

In some embodiments, an intermittent or superimposed directional magnetic field may be used to position the nanoparticles in the targeted area. In some embodiments, intermittent or superimposed directional magnetic field may be used to hold the nanoparticles in position at the target area, thereby reducing risk associated with systemic dispersion.

In some embodiments, the method of the present invention further comprises administering an effective amount of one or more therapeutic agents to the subject. In some embodiments, the one or more therapeutic agents comprise one or more antimicrobial agent. In some embodiments, the one or more antimicrobial agents are one or more selected from the group consisting of one or more antiseptics, one or more antibiotics, one or more antibacterials, one or more antifungals, one or more anti-acanthmoebics, and one or more chemotherapeutics. In some embodiment, one or more antibiotics may be administered. The antibiotics are selected based on their known effectiveness against the microorganisms known to associate with infection or biofilm formation or against the microorganisms identified from the subject. Examples of widely used antibiotics include, but are not limited to, oxacillin, vancomycin, cephalexin, gentamicin, augmentin and amikacin.

In some embodiments, one or more antiseptics are used. Examples of antiseptics include, but are not limited to, alcohols, quaternary ammonium compounds, chlorhexidine, diguanides, antibacterial dyes, chlorine, hypochlorites, inorganic iodine compounds, metals, peroxides, permanganates, halogenated phenol derivatives, and quinolone derivatives.

The antimicrobial agents may be administered by any route known to one skilled in the art. Examples of routes of administration include, but are not limited to, intra-respiratory, nasal, topical, oral, intravenous, intraperitoneal, intramuscular, transmucosal, buccal, rectal, vaginal, and sublingual.

In some embodiments, the at least one therapeutic agent is administered by any suitable route known to one skilled in the art. Examples of routes of administration include, but are not limited to, intra-respiratory, nasal, topical, oral, intravenous, intraperitoneal, intramuscular, transmucosal, buccal, rectal, vaginal, or sublingual.

In some embodiments, the administration of the at least one therapeutic agent occurs at any suitable time with respect to administration of the nanoparticles and positioning in electromagnetic field. In some embodiments, the administration of the at least one therapeutic agent occurs at least partially simultaneously with at least one of administration of the nanoparticles and positioning in electromagnetic field. In some embodiments, the administration of the at least one therapeutic agent occurs before administration of the nanoparticles and positioning in electromagnetic field. In some embodiments, the administration of the at least one therapeutic agent occurs after administration of the nanoparticles and positioning in electromagnetic field.

In some embodiments, the at least one therapeutic agent is administered to a subject as frequently as is required. In some embodiments, the at least one therapeutic agent is administered several times daily, as once a day, once every two days, once every three days, once every four days, once every five days, once every 6 days, once a week, once every two weeks, once every three weeks, once every four weeks, once a month, once every two months, once every three months, once every four months, once every five months, once every six months, once every seven months, once every eight months, once every nine months, once every 10 months, once every 11 months, or once a year. The frequency of the dose will be readily apparent to the skilled artisan and will depend upon any number of factors, including, but not limited to, age of the subject, the weight of the subject, the sex of the subject, the type of infection, and the severity of the infection. In some embodiments, the dose of the at least one therapeutic agent is any suitable range known to one skilled in the art.

In some embodiments, the method of the present invention is performed in any suitable location. In some embodiments, the method is performed in vitro. In some embodiments, the method is performed in vivo.

In some embodiments, the present invention provides a method of treating and/or preventing an infection comprising the steps of administering an effective amount of at least one therapeutic agent to a subject and positioning the subject in an electromagnetic field.

In some embodiments, the administration of the at least one therapeutic agent occurs at any suitable time with respect to administration of the nanoparticles and positioning in electromagnetic field. In some embodiments, the administration of the at least one therapeutic agent occurs at least partially simultaneously with at least one of administration of the nanoparticles and positioning in electromagnetic field. In some embodiments, the administration of the at least one therapeutic agent occurs before administration of the nanoparticles and positioning in electromagnetic field. In some embodiments, the administration of the at least one therapeutic agent occurs after administration of the nanoparticles and positioning in electromagnetic field.

### System

In another aspect, the present invention provides a system configured to disrupt a biofilm mass. In some embodiments, the system comprises a composition having at least one composition or nanoparticle of the disclosure and a device configured to irradiate a subject with an electromagnetic field to activate the at least one nanoparticle or composition and disrupt the biofilm mass. In some embodiments, the system further comprises at least one therapeutic agent. In some embodiments, the at least one therapeutic agent is a part of the composition. In some embodiments, the at least one therapeutic agent is administered separately.

In some embodiments, the device configured to irradiate the subject with electromagnetic radiation comprises one or more Helmholtz coils, a signal generator, and a power supply. In some embodiments, the system comprises a support for the one or Helmholtz coils. In some embodiments, the support is a shelved rack. In some embodiments, the shelved rack is acrylic. In some embodiments, the system comprises an acrylic shelved rack with wrap-around Helmholtz coils, a signal generator, and a power supply, wherein the power supply acts as the energy source to supply an adequate intensity electromagnetic field to the subject to produce the desired effect, while the signal generator allows for customized control of the magnetic field applied to the subject, and the wrap-around Helmholtz coils act to physically produce the field, and are arranged so the field is uniform within a defined area. Examples of parameters the signal generator controls include, but are not limited to, selection between an array of waveforms, selection between a pulsed vs. static field, custom frequency, and amplitude selection.

In some embodiments, the device further comprises a digital oscilloscope configured to allow monitoring of the signal waveform.

In some embodiments, the device maintains the magnetic nanoparticles in a localized area by continuously exerting a very mild and constant magnetic field, thereby avoiding dispersion of the nanoparticles systemically, reducing dosage and increasing efficiency and efficacy.

In some embodiments, the device is positioned external to the targeted site. In some embodiments, the device is wearable and positioned to produce an electromagnetic field only around the targeted site.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out exemplary embodiments of the present invention, and are not to be construed as limiting in any way the remainder of the disclosure.

### Example 1: Pulsed Electromagnetic Fields Eradicate Staphylococcus epidermidis Biofilms and Synergistically Enhance the Anti-Biofilm Efficacy of Antibiotics

Staphylococcus epidermidis is a Gram-positive bacterium implicated in a multitude of clinical infections and brings about significant issues for the healthcare field. Specifically, S. epidermidis contributes to a high percentage of burn wound infections and is the leading cause of surgical-site infections (SSIs) across multiple specialties, including orthopedics and neurosurgery (Hubab, M. et al., 2020, J Burn Care Res, 41(6):1207-1211; López Pereira, P. et al., 2017, Br J Neurosurg, 31(1):10-15). S. epidermidis-based SSIs are the most recurrent cause of nosocomial sepsis (Nguyen, T. H. et al., 2017, Front Cell Infect Microbiol, 7:90) and is the most frequent cause of death in post-operative hospitalized surgical patients (Otto, M., 2017, Future Microbiol., 12(12):1031-1033). S. epidermidis is the also leading cause of medical device infections (Oliveira, W. F. et al., 2018, J Hosp Infect, 98(2):111-117; Perez, K. et al., 2018, Infect Immun, 86(10)). Specifically, S. epidermidis is a major contributor to periprosthetic joint infections (PJI), (Morgenstern, M. et al., 2016, J. Orthop. Res., 34(11):1905-1913) which are, in turn, the leading cause of revision in total knee arthroplasty (TKA) (Koh, C. K. et al., 2017, Clin Orthop Relat Res, 475(9):2194-2201). Said revisions cost the healthcare industry over $450 million annually, (Parvizi, J. et al., 2010, J Arthroplasty, 25(6 Suppl):103-107) and are an average of $60,000 more expensive than revision due to aseptic loosening, the second leading cause of TKA failure (Parvizi, J. et al., 2010, J Arthroplasty, 25(6 Suppl):103-107). The current practice for a TKA revision due to PJI is a two-stage procedure requires two separate, heavily invasive operations—the first being particularly invasive because of the involved heavy debridement of the infected tissue (Gehrke, T. et al., 2015, Bone Joint J, 97-b(10 Suppl A):20-29). This procedure also has a high mortality risk resulting from related S. epidermidis-based sepsis. This highlights the need effective novel treatment, vastly improving the morbidity and mortality of countless patients undergoing a TKA revision due to S. epidermidis infection.

The propensity of S. epidermidis to form biofilms, agglomerated groups of cells adhering to a surface and one another, encased in a self-produced extracellular polymeric substance (EPS), (Paharik, A. E. et al., 2016, Microbiol Spectr, 4(2); Buttner, H. et al., 2015, Front Cell Infect Microbiol, 5:14) is what makes it a particularly dangerous pathogen. Biofilms tend to form on surfaces such as bones and medical implants, causing infections like osteomyelitis and PJI, respectively (Paharik, A. E. et al., 2016, Microbiol Spectr, 4(2); Buttner, H. et al., 2015, Front Cell Infect Microbiol, 5:14; Morgenstern, M. et al., 2016, J Orthop Res, 34(11):1905-1913). Biofilm formation further serves to worsen S. epidermidis-based infections because once fully established, small groups of aggregated cells may break off and colonize other parts of the body, spreading the infection (Paharik, A. E. et al., 2016, Microbiol Spectr, 4(2)). The most significant problem posed by S. epidermidis biofilm formation is its intrinsic ability to resist antibiotic treatment and the host's natural immune response (Davies, D., 2003, Nat Rev Drug Discov, 2(2):114-122; Ma, Y. et al., 2012, Antimicrob Agents Chemother, 56(11):5923-5937). The primary mechanism by which biofilms resist antibiotic treatment is limiting the antibiotic's ability to penetrate into the EPS that makes up the biofilm (Hall, C. W. et al., 2017, FEMS Microbiol Rev, 41(3):276-301). Cells deeply encased in a biofilm also become less metabolically active, making them less susceptible to both antibiotic and immunity-based therapies. Biofilm-derived antibiotic resistance has a devastating effect on patient outcomes; Morgenstern et al. showed that biofilm-forming S. epidermidis isolates led to a 24% lower infection cure rate than non-biofilm forming isolates, (Morgenstern, M. et al., 2016, J Orthop Res, 34(11):1905-1913) further highlighting the need for a novel treatment by which to combat S. epidermidis biofilm infections.

The present study investigated high-frequency pulsed electromagnetic fields (PEMF) as a potential treatment to S. epidermidis biofilms. PEMF is an emerging, but poorly understood treatment technique throughout the medical field, having been shown to improve bone fracture healing and as a possible treatment for cancer, rheumatoid arthritis, and osteoarthritis, among other biomedical applications (Vadali, M. Et al., 2016, Cancer Med, 5(11):3128-3139; Ross, C. L. et al., 2019, Front Immunol, 10:266; Yang, X. Et al., 2020, Phys Ther, 100(7):1118-1131; Mohajerani, H. Et al., 2019, J Stomatol Oral Maxillofac Surg, 120(5):390-396). Moreover, Faveri et al. showed that miniaturized electromagnetic devices inhibited polymicrobial biofilm formation on dental implants (Faveri, M. et al., 2020, Biofouling, 36(7):862-869). This antimicrobial potential, along with PEMF's non-invasive nature, makes it a strong candidate to be an eventually successful S. epidermidis biofilm infection treatment. PEMF has also been shown to stimulate the proliferation of osteoblast-like cells (Miyamoto, H. et al., 2019, Bioelectromagnetics, 40(6):412-421), enhance osteogenesis (Benya, P. D. et al., 2021, PLoS One, 16(2):e0244223), and significantly improve wound healing (Patino, O. et al., 1996, J Burn Care Rehabil, 17(6 Pt 1):528-531). Thus, a PEMF-based treatment could simultaneously exhibit the intended anti-biofilm effect while also stimulating bone growth and wound healing.

The present study hypothesized that: (1) S. epidermidis biofilm formation and planktonic cell growth would be decreased by the length of exposure to PEMF, (2) the viability of preformed S. epidermidis biofilms would also be decreased by the length of exposure to PEMF, and (3) the antibiotic efficacy in treating biofilm infections would be enhanced by the length of exposure to PEMF. The expectation for hypotheses one and two was that PEMF would affect biofilm formation and strength by interfering with the attractive electrostatic forces between S. epidermidis cells and the surface to which they adhere. This potential disruption of the biofilm stability and structure by PEMF may open pores in the biofilm matrix, allowing antibiotics to penetrate the matrix layer and directly affect bacterial cells, leading to hypothesis three. This rationale is further supported by studies from Pickering et al. (Pickering, S. A. et al., 2003, J Bone Joint Surg Br, 85(4):588-593), Matewele (Matewele, P. et al., 2010, J Microbiol Methods, 83(2):275-276), and Esfahani et al. (Esfahani, A. et al., 2019, J Appl Microbiol, 126(1):87-101), which suggest PEMF has the potential to augment the efficacy of alternative antimicrobial therapies.

These hypotheses were investigated using an in vitro approach, in which S. epidermidis biofilms were grown on plastic cell-culture treated plates and exposed to varying durations of PEMF. PEMF not only significantly inhibited S. epidermidis biofilm and planktonic cell growth, but also eradicated preformed biofilms. Moreover, when oxacillin or vancomycin were simultaneously administered at their minimum inhibitory concentration (MIC), the data showed that PEMF synergistically enhanced the biofilm eradication ability of both antibiotics, further suggesting PEMF's ability to alter S. epidermidis biofilm strength and structure.

The materials and methods employed in these experiments are now described.

### Materials

Experiments were performed initially on S. epidermidis strain ATCC 14990 and further tested on S. epidermidis strain ATCC 35984, which is known to be a particularly strong biofilm former (Okajima, Y. et al., 2006, Invest Ophthalmol Vis Sci, 47(7):2971-2975). Trypticase soy broth (TSB) (30 g l⁻¹ in water, autoclaved at 121° C. for 15 minutes; BD Bacto TSB from Fisher Scientific) was used for in vitro growth of bacterial cell cultures, and 50% glucose was added to the medium at a final concentration of 0.25%. Sterile Falcon 6-well, clear, flat-bottom, tissue culture-treated, cell culture plates (Corning; Lot #1059012) and sterile CELLSTAR cell culture 24-well, PS, clear, tissue culture-treated plates (Greiner; Lot #E100501A) were used for S. epidermidis cell plating and biofilm growth (6-well plates used for crystal violet assay experiments; 24-well plates used for alamarBlue assay experiments).

Crystal violet (CV) stain (0.41% w/v in 12% ethanol; Thermo Fisher Scientific, Lot #V70290) and alamarBlue cell viability reagent (Invitrogen, Lot #2263437) (used as 5.0% alamarBlue in TSB) were used during biofilm growth and viability quantification, both of which are established measures of S. epidermidis biofilm viability (Esfahani, A. et al., 2019, J Appl Microbiol, 126(1):87-101; Hamon, M. A. et al., 2001, Mol Microbiol, 42(5):1199-1209; Karau, M. J. et al., 2020, Spine Deform, 8(4):553-559; Chopra, R. et al., 2017, Sci Rep, 7(1):7520; Müller, C. et al., 2010, Langmuir, 26(6):4136-4141; Hartvig, R. A. et al., 2011, 27(6):2634-2643; Petrova, O. E. et al., 2012, J Bacteriol, 194(10):2413-2425; Nakanishi, E. Y. et al., 2021, Sustainability, 13(11):5836; Pettit, R. K. et al., 2005, Antimicrob Agents Chemother, 49(7):2612-2617). Wash buffer was used to rinse medium and planktonic S. epidermidis cells from all plated wells to ensure only biofilm-adhered cells remained before quantification (Hamon, M. A. et al., 2001, Mol Microbiol, 42(5):1199-1209).

Oxacillin (Sigma Aldrich, Source #1B190146, Batch #0000089022) and vancomycin (Sigma Aldrich, Lot #048M4087V) were the two antibiotics used to test potential synergism with PEMF treatment.

The PEMF apparatus was custom made in the laboratory and was previously described in Ghalayani Esfahani et al. 2019 (Esfahani, A. et al., 2019, J Appl Microbiol, 126(1):87-101). Briefly, the PEMF apparatus consisted of an acrylic shelved rack with wrap-around Helmholtz coils, a signal generator, and a power supply. The signal had a characteristic frequency of 40 kHz and was characterized by a square waveform for all trials. The intensity of the magnetic field was measured at various points on the rack with a Gaussian probe, and the cell culture plates were positioned where the field had a constant intensity. A digital oscilloscope (TBS1000C, Tektronix, Beaverton, OR) was used to monitor the signal waveform. All plates not under PEMF treatment were kept in a 37° C. incubator, identical temperature to the PEMF incubator.

### Biofilm Growth

S. epidermidis biofilms were grown by initially incubating freezer stock S. epidermidis cell scrapings in TSB media at 37° C. for 24 hours with shaking. Subsequently, the cells were diluted 1:100 into TSB media containing 0.25% glucose and then aliquoted, in 3 ml or 1 ml aliquots, into 6-well or 24-well plates, respectively. To test the ability of PEMF to disrupt pre-formed biofilms, inoculated plates were incubated for 24 hours to allow biofilm formation to occur. Negative controls of only TSB media containing 0.25% glucose (i.e., no cells) were also included for all plates to ensure no contamination occurred.

### Biofilm Quantification

Crystal Violet (CV) assay was utilized to quantify biofilm formation in vitro as previously described (Esfahani, A. et al., 2019, J Appl Microbiol, 126(1):87-101). After incubation of the biofilm in the plates with or without treatment, the media and any non-adhered cells were removed. The well of the plates were then washed with Wash Buffer three times to ensure only biofilm-adhered cells remained. Next, wells were incubated with CV at room temperature for 15 minutes. The CV stain was then removed, and wells were washed twice with water. An 80% ethanol/20% acetone solution was then added and allowed to incubate for 20 minutes at room temperature to solubilize the dye. Absorbance readings were thereafter obtained at 570 nm and recorded for each well (BMG LABTECH, FLUOstar Omega plate reader).

AlamarBlue was utilized to measure the viability of biofilm cells. The alamarBlue assay was performed by removing the growth media and washing all wells with Wash Buffer as stated above for the CV assay. A 5% alamarBlue solution in TSB media containing 0.25% glucose was next added to the wells. Fluorescence readings were then obtained 30 minutes after the addition of alamarBlue. For this assay, a plate reader was set to well-scanning, bottom reading optic mode. The gain was set at 1100, and fluorescence was read at 544/590 nm (excitation/emission). Samples underwent 10 seconds of shaking at 100 rpm directly prior to each reading.

### Planktonic Cell Quantification

Colony-forming units per ml (CFU/ml) counts were used to quantify the number of planktonic S. epidermidis cells post-treatment. Planktonic cells were first diluted serially, and 20 μl aliquots from the 10⁻² to 10⁻⁵ dilution were then spread on Luria Broth (LB) plates and allowed to grow overnight before CFU counts were taken the following day. CFU/ml was then calculated for each using Equation (1) as follows: CFU/ml=(number of colonies×dilution factor)/volume.

### Determination of Antibiotic Minimum Inhibitory Concentrations (MIC)

To test possible synergism between PEMF exposure and antibiotic treatment, the MIC of both oxacillin and vancomycin for the specific S. epidermidis strain used was determined. S. epidermidis were first incubated in TSB media at 37° C. for 24 hours with shaking. The culture was subsequently diluted 1:100 into TSB media containing 0.25% glucose, and 100 μl aliquots of the diluted cells was added to 96-well cell culture plates. The antibiotic being tested was then serially diluted down the plate, resulting in twelve final concentrations to be tested, ranging from 500 μg/ml to 0.25 μg/ml. After 24 hours, OD₆₀₀ readings of the wells were taken. The MIC of each antibiotic was determined to be the lowest concentration to show inhibition of planktonic S. epidermidis cell growth.

### Biofilm Fixation and SEM Imaging

The structure of the S. epidermidis biofilms were determined using scanning electron microscope (SEM). Biofilms were grown and treated on tissue culture-treated plastic slides cut from 24-well plates and placed within the 24-well cell culture plates before bacterial cultures were inoculated. These slides were cut so SEM imaging could be performed on an equivalent surface to that used in all quantitative experiments without disturbing the biofilm sample during the imaging process. Fixation was performed by initially washing all slides twice in phosphate-buffered saline (PBS), then incubating them in 2.5% glutaraldehyde for 48 hours. At the conclusion of the 48-hour fixation period, slides were rinsed in PBS again for two cycles of 10 minutes each. This step was followed by a 1-hour post-fixation in OsO₄ (osmium tetroxide). The samples were then dehydrated using an ascending ethanol series where samples were incubated in 25% ethanol, 50% ethanol, 75% ethanol, and then finally 97% ethanol for 30 minutes each. All samples were then dried overnight, sputter-coated, and imaged in the SEM.

### Reproducibility and Statistical Analyses

To confirm the reproducibility of results, all experiments were run identically at least three times with similar outcomes before results were analyzed and reported. All groups were compared using 1-Way ANOVA with Tukey post-hoc tests on SPSS statistics, with statistical significance defined as p≤0.05. One major comparison of interest was the theoretical additive effect of PEMF and antibiotic treatment vs. the empirical effect of simultaneous PEMF and antibiotic treatment. The theoretical additive effect was calculated based on the effect of the antibiotic treatment alone and PEMF exposure alone. Equation (2), a simple probability calculation, was used as such: P(A)+(1−P(A))*P(B); with A representing the percent biofilm reduction by PEMF alone and B representing reduction by antibiotic alone. The data arising from this calculation was compared to the empirical data collected from simultaneous PEMF and antibiotic treatment experimental groups to determine if statistically significant synergism had occurred.

The results of these experiments are now described.

### Pemf Inhibits Biofilm Formation and Planktonic Cell Growth

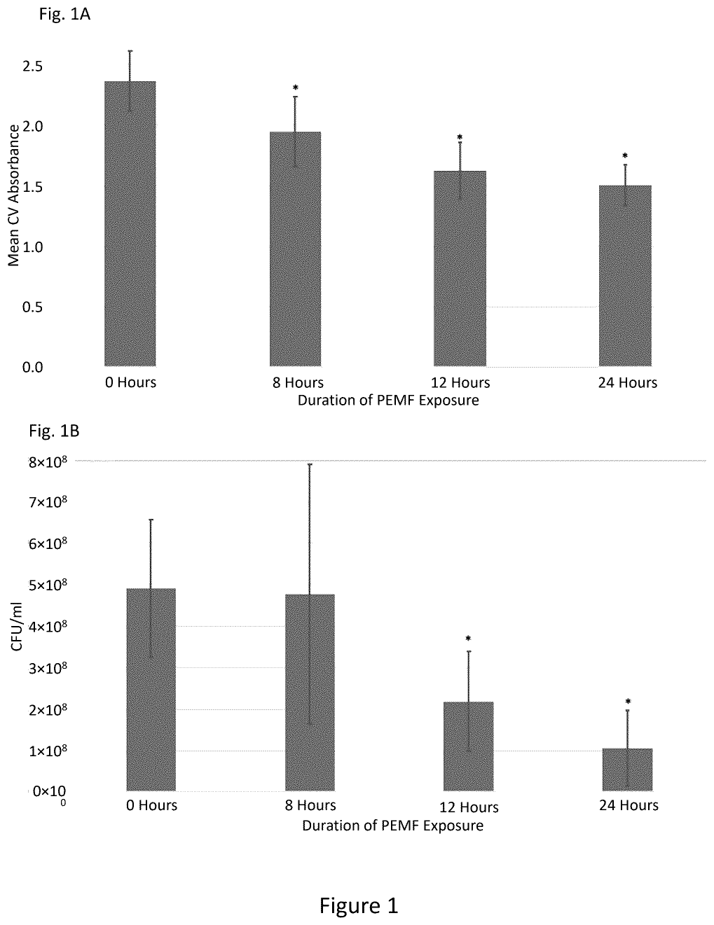

The initial hypothesis tested was that PEMF would disrupt S. epidermidis surface adhesion and biofilm formation. S. epidermidis cells were inoculated into conditions that support biofilm formation and they were exposed to PEMF for varying durations (4 hours×2 cycles, 12 hours consecutively, or 24 hours consecutively) or received no PEMF treatment. The degree of biofilm formation was quantified using a Crystal Violet Assay, PEMF exposure significantly inhibited both S. epidermidis biofilm formation (n=36, p<0.001, F=23.066, df=3) (FIG. 1A and FIG. 1). PEMF significantly inhibited biofilm formation at all exposure durations, inhibiting formation by up to 35% at 24 hours of exposure (p<0.001). These results indicate that PEMF inhibits the ability of S. epidermidis to form biofilms.

To determine whether PEMF was able to specifically disruption biofilm formation or disrupted the ability of cells to grow, the impact of PEMF on planktonic cell growth is measured. (n=36, p<0.001, F=8.618, df=3). Planktonic cell growth was inhibited by 60% after 12 hours (p=0.029) and 80% after 24 hours (p=0.001) of PEMF exposure. These data indicate that PEMF is toxic to S. epidermidis cells and its ability to disrupt biofilm formation is likely related to this cellular toxicity activity.

PEMF Eradicates Pre-Formed S. epidermidis Biofilms

Most infection patients do not show symptoms or undergo treatment until a biofilm has formed at the infected site, thus, PEMF's ability to eradicate a preformed S. epidermidis biofilm was tested. It was hypothesized here that PEMF would weaken bacterial cells' affinity to adhere to a surface and one another, breaking down the biofilm EPS and reducing total biomass. To test this hypothesis, S. epidermidis cells were grown as described above, except the cells were then allowed to grow for 24 hours untreated at 37° C. to form a full biofilm before being exposed to PEMF. The amount of biomass was measured using the CV assay.

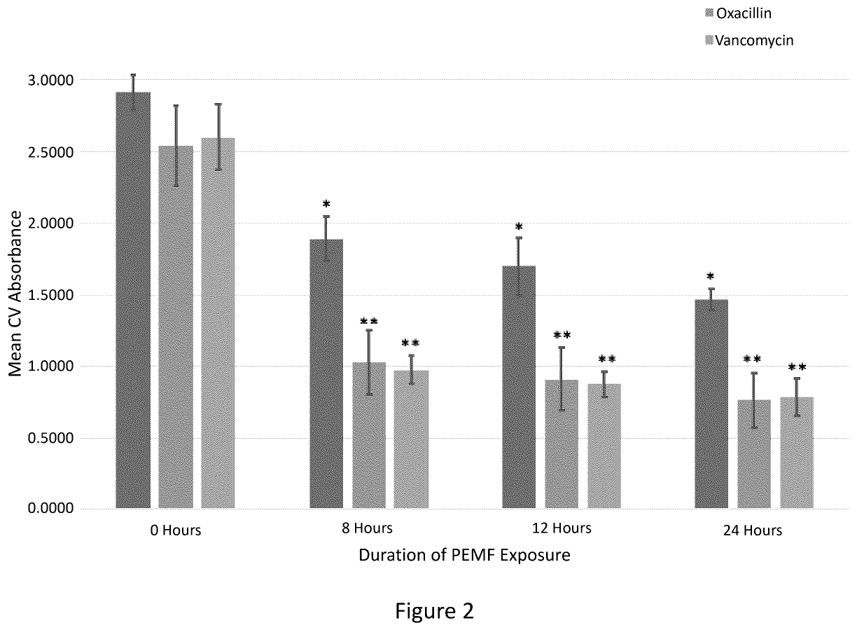

As hypothesized, PEMF significantly eradicated preformed S. epidermidis biofilms at all three exposure durations (FIG. 2). The 24-hour PEMF exposure group showed the largest, 53% biofilm reduction (p<0.001) compared to the sample that received no PEMF treatment and 66% when quantified by alamarBlue assay.

PEMF Enhances the Efficacy of Antibiotics Against S. epidermidis Biofilms

Based on PEMFs ability to break down preformed biofilms, it was further hypothesized that PEMF would enhance the efficacy of antibiotics by creating more direct access for antibiotics to S. epidermidis cells. This was tested by adding antibiotics, oxacillin, and vancomycin, at each PEMF exposure duration, using pre-determined antibiotic minimum inhibitory concentrations. The amount of biofilm was again quantified by CV assay and the amount of viable cells in the biofilm was determine by alamarBlue assay.

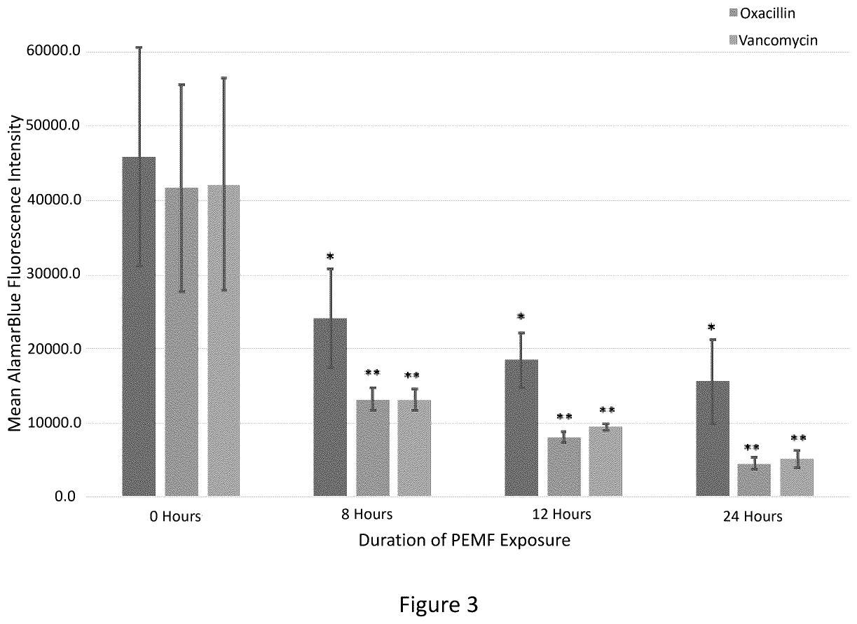

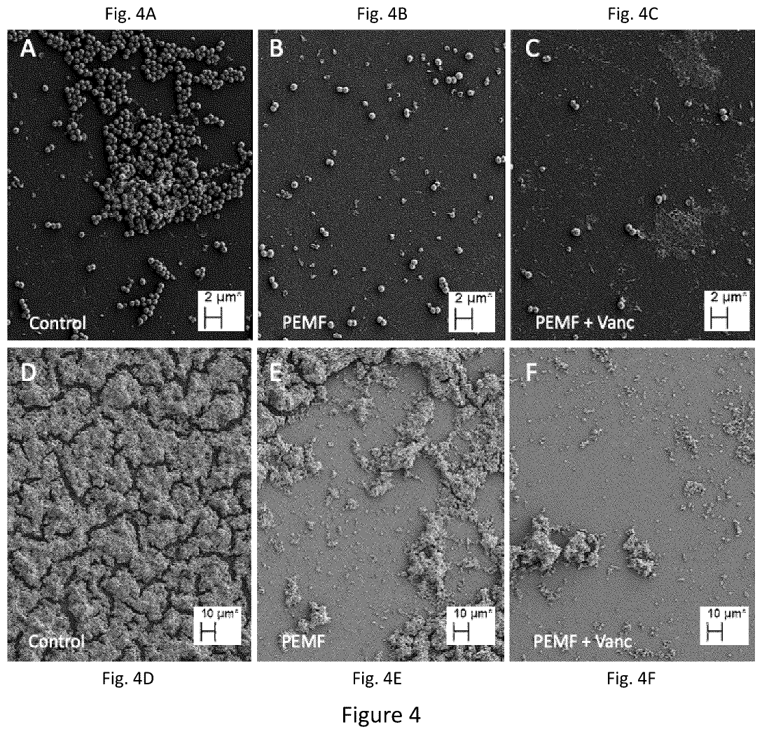

Neither oxacillin nor vancomycin (FIG. 2, FIG. 3) alone significantly eradicated the S. epidermidis biofilm. This empirically combined treatment of PEMF exposure and antibiotics proved to be significantly synergistic as compared to the theoretical additive effect at all time points for both oxacillin and vancomycin (oxacillin trials: p<0.001 n=54, F=33.640, df=5; vancomycin trials: p<0.001 n=54, F=33.715, df=5). Furthermore, the largest mean biofilm reduction seen compared to the 48-hour control group was the PEMF (24-hour exposure duration) and oxacillin simultaneous treatment group, showing a 74% reduction of total biomass on average, quantified by CV assay, and a 89% reduction in viable biofilm adhered cells, quantified by alamarBlue assay (as seen in FIG. 2 and FIG. 3). These results were further confirmed through SEM imaging. SEM results comparing staphylococcal samples with and without PEMF treatment or with a combination of PEMF and vancomycin are shown in FIG. 4A through FIG. 4F.

### Discussion

S. epidermidis remains a major contributor to many devastating clinical infections, particularly surgical site infections and orthopedic implant infections, such as PJI and spinal implant infections (Morgenstern, M. et al., 2016, J Orthop Res, 34(11), 1905-1913; Karau, M. J. et al., 2020, Spine Deform, 8(4):553-559). Biofilm formation significantly increases the morbidity and mortality of such infections by increasing bacterial resistance to antibiotics and the host immune response (Paharik, A. E. et al., 2016, Microbiol Spectr, 4(2); Buttner, H. et al., 2015, Front Cell Infect Microbiol, 5:14; Morgenstern, M. et al., 2016, J Orthop Res, 34(11), 1905-1913). As there is currently no FDA-approved treatment to eradicate established biofilm infections, a novel solution would provide massive value to surgeons and patients alike. This study successfully demonstrated a novel approach to eradicating such infections, using PEMF to eradicate the biofilm on its own and to work synergistically with multiple antibiotics.

The data revealed that PEMF can not only inhibit S. epidermidis biofilm formation and eradicate preformed biofilms, but when combined with antibiotic exposure it can eradicate up to 89% of an established Staphylococcal biofilm. The ability of PEMF to eradicate biofilms carries much more weight towards an eventual clinical solution than does biofilm inhibition, as clinical infections are more than likely have already formed a full biofilm by the time a patient presents with symptoms. Much of the importance of this novel approach lies in its non-invasive nature, which, if integrated clinically, would greatly decrease both cost and patient discomfort compared to the current two-stage revision technique (Zimmerli, W. et al., 2004, N Engl J Med, 351(16):1645-1654; Lenguerrand, E. et al., 2019, Lancet Infect Dis, 19(6):589-600). Similar success has been shown using alternating magnetic field therapy by Chopra et al. in 2017 and Wang et al. in 2021; however, important differences are notable. Both previous studies used an alternating magnetic field to eradicate biofilms by generating heat in a metal implant, essentially burning the bacteria (Chopra, R. et al., 2017, Sci Rep, 7(1):7520; Wang, Q. et al., 2021, NPJ Biofilms Microbiomes, 7(1):68). While successful, the heat produced may also affect surrounding tissue and be detrimental to the osseointegration of an orthopedic implant. On the other hand, the presented study produced similar eradication effects while using a low enough frequency pulsed magnetic field to avoid any concerns about heat being produced and damaging tissue.

The mechanism by which PEMF eradication S. epidermidis is currently unknown. It is suspected that much of the effect is due to prolonged PEMF exposure disrupting electrostatic interactions between bacteria and the growth surface, and between adjacent Staphylococcal cells. This hypothesis is supported by work done by Muller et al. and Hartvig et al., among others, proving that electrostatic interactions play a major role in biofilm attachment and strength (Müller, C. et al., 2010, 26(6):4136-4141; Hartvig, R. A. Et al., 2011, Langmuir, 27(6):2634-2643; Petrova, O. E. et al., 2012, J Bacteriol, 194(10):2413-2425; Nakanishi, E. Y. et al., 2021, Sustainability, 13(11):5836). Further, much of biofilm-based antibiotic resistance is caused by the biofilm matrix and structure essentially blocking antibiotics' access to bacterial cells, physically limiting their capability to have any effect on the cells causing infection (Davies, D., 2003, Nat Rev Drug Discov, 2(2):114-122; Ma, Y. et al., 2012, Antimicrob Agents Chemother, 56(11):5923-5937; Hall, C. W. et al., 2017, FEMS Microbiol Rev, 41(3):276-301). Thus, the synergistic effect of PEMF and antibiotics was likely due to the weakened electrostatic forces, resulting in weak biofilm surface attachment, opening pores in the biofilm, or weakening the structure enough to give antibiotics direct access to S. epidermidis cells, inducing bactericidal and biofilm eradication effects.

With such promising results, the limitations of this study are important to note. This study was performed using only cell-culture-treated polystyrene plates, limiting its direct generalizability to the various metal alloys used in orthopedic implants. However, because cell-culture treated plates were used and it has been well-established that Staphylococcal biofilms grow well on multiple plastic and metal surfaces, it was assumed that this model is well representative of PEMFs interaction with biofilm on a variety of surfaces. Additionally, only two strains of S. epidermidis were tested in the presented study. While it is encouraging that PEMF and antibiotic treatment showed similar results on both strains, it is important to test this treatment on other clinically relevant bacteria, such as Staphylococcus aureus and Pseudomonas aeruginosa, along with multispecies biofilms. Finally, specific limitations exist with any in vitro infection model, as both biofilm growth and PEMF presumably act differently when exposed to a flow state and natural immune factors in human and animal blood. While such challenges must be addressed en route to any in vivo or clinical studies, the presented data still makes a strong case for PEMF's potential as an anti-biofilm therapy.

Despite these limitations, it is believed that this data establishes PEMF as a viable method by which to prevent and eradicate staphylococcal biofilm infections. As stated, a clinical therapy derived from this technique would be a major improvement over the current two-stage revision process for surgeons and patients alike. Moreover, PEMFs shown ability to act synergistically in conjunction with antibiotics suggests its potential to do the same with other antimicrobial and antibiofilm therapies, such as magnetic nanoparticles, synthetic antibodies, antimicrobial peptides, and a multitude of other therapies currently being tested, cementing its importance for inclusion in future research in the field.

### Example 2: Control of Periprosthetic Joint Infection

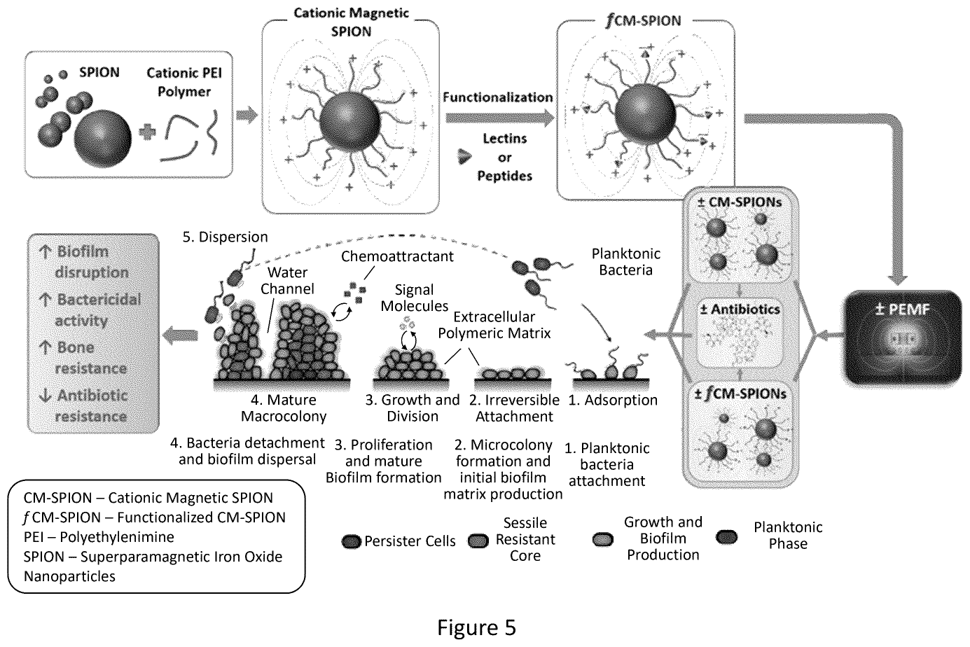

The proposed study aims at engineering a novel, non-invasive method to disrupt orthopedic biofilm-based infections by using a combination of functionalized iron-core (magnetic) nanoparticles (briefly functionalized nanoparticles, fNPs) and pulsed electromagnetic field (PEMF) (FIG. 5).

### BACKGROUND

Prosthetic joint infection (PJI) is a serious complication of hip and knee replacement. Studies project that PJI will dramatically increase over the next two decades compared with other modes of orthopedic implant failure, with some anticipating over 60% of all orthopedic implants will need revisions due to infection (Kurtz, S. M. et al., 2012, J Arthroplasty, 27(8):61-65; Kurtz, S. M. et al., 2008, J Arthroplasty, 23(7):984-991).

Bacterial adhesion to the orthopedic implant is the first and most important step in PJI. It is a complex process influenced by environmental factors, bacterial properties, material surface properties, and the presence of serum or tissue proteins. Properties of the substrate, such as the chemical composition of the material, surface charge, hydrophobicity, surface roughness, and specific proteins at the surface, are all considered important in the initial cell attachment process. If bacterial adhesion occurs before tissue regeneration, host defenses often cannot prevent surface colonization for certain bacterial species capable of forming a protective biofilm layer that shields the bacteria from the immune responses and antibiotic treatment (Arciola, C. R. et al., 2018, Nat Rev Microbiol, 16(7):397-409; Yan, J. et al., 2019, Elife, 8). These biofilms of bacterial cells encased in an extracellular polymeric matrix are particularly prone to form on implanted medical devices, (Limoli, D. H. et al., Microbiol Spectr, 3(3):3-3; Toler-Nielsen, T. 2015, Microbiol Spectr, 3(2):3-2; Chang, H-J. et al., 2018, ACS Synth Biol, 7(1):166-175) as these abiotic surfaces lack endogenous mechanisms to resist bacterial adherence and biofilm formation (Zimmerli, W. et al., 2017, APMIS, 125(4):353-364). At present, treatment of biofilm infections on devices, such as orthopedic implants, requires invasive and often complex surgeries, extraction of the infected implant (Trampuz, A. et al., 2006, Injury, 37(2):S59-S66; Antony, S. et al., 2016, Infect Disord Drug Targets, 16(1):22-27) followed by extensive antibiotic prophylaxis, and eventually reoperation and insertion of a new implant.

Among all pathogens in orthopedic implant infections, Staphylococcus epidermidis and aureus are the most common and comprise up to two-thirds of cases (Zimmerli, W. et al., 2017, APMIS, 125(4):353-364; Ribeiro, M. et al., 2012, Biomatter, 2(4):176). They are the principal causative agents of two major types of infection affecting bone: septic arthritis and osteomyelitis, which involve the inflammatory destruction of joint and bone.

### Preliminary Studies:

It has been previously shown that a Pulsed Electromagnetic Field (PEMF) could decrease the ability of Staphylococcus aureus and epidermidis to form biofilms (Esfahani, A. et al., 2019, J Appl Microbiol, 126(1):87-101). This inhibition was observed in combination with surfaces that release gallium, a toxic metal that substitutes iron. However, gallium has a relatively short time frame in which to act, and there is a need to develop methods to disrupt biofilms once they have formed on indwelling medical devices.

### Hypothesis and Rationale:

It was hypothesized that a) the combination of fNPs with PEMF mechanically disrupt the biofilm by decreasing the viability of the bacterial cells, b) the functionalization of NPs surface with lectin increases the ability of the nanoparticles to bind the biofilm, thus increasing the efficacy and efficiency of the PEMF-guided mechanical disruption, and c) the functionalization with quorum sensing (QS) signaling molecules increases the specificity of binding to specific bacteria strain further increasing the efficacy and efficiency of the disruption.

The general idea was that magnetic nanoparticles (NPs) placed in oscillation by the magnetic field have a mechanical-disrupting effect on the biofilm. This effect per se could be sufficient to detach the biofilm from the infected surface, break it down to make it more susceptible to the native immune system and antibiotics, and finally allow the surface to be colonized and coated by other cells (for example, osteoblast) that would further prevent any further biofilm formation over the surface of the device. Due to their small size, iron oxide nanoparticles may penetrate deep into the biofilm. The magnetic field allows the manipulation of movement of the nanoparticles by exerting mechanical force and generating heat to disrupt the integrity of biofilm structures. Furthermore, the surface of NPs can be easily modified to increase electrostatic or covalent binding with antibiotics, peptides, proteins, and bacteria. In addition, iron oxides could generate reactive oxygen species by reacting with hydrogen peroxide via Fenton reaction, which in turn may contribute to the antimicrobial activity. Besides having the effect of placing the particles in oscillation, PEMF may also increase the penetration of the NPs in the biofilm by electroporation.

Biofilms are coated with a self-generated extracellular matrix composed of exopolysaccharides, proteins, and DNA (Kurtz, S. M. et al., 2008, J Arthroplasty, 23(7):984-991; Okshevsky M. et al., 2015, Crit Rev Microbiol, 41(3):341-352; Okshevsky M. et al., 2015, Curr Opin Biotechnol, 33:73-80; Erskine, E. et al., 2018, J Mol Biol, 430(20):3642-3656; Limoli, D. H. et al., 2015, Microbiol Spectr, 3(3)). Coating the NPs with a lectin that binds sugars, such as those in the exopolysaccharides of the biofilm matrix, should increase the concentrations of the nanoparticles at the biofilm (Neu, T. R. et al., 1999, 310:145-152; Breitenbach, Barroso Coelho LC, 2018, J Appl Microbiol, 125(5):1238-1252). To further increase the specificity, these NPs can be coated with quorum sensing molecules, such as the autoinducing peptide (ATP) of Staphaureus (20-24)(Vasquez, J. K. et al., 2019, ACS infectious diseases 5(4):484-492; Vasquez, J. K. et al., 2017, ChemBioChem 18(4):413-423; Tan, Li et al., 2018, Frontiers in microbiology 9:55; Jenul, C. et al., 2018, Microbiol Spectr 6:GPP3-0031-2018; Murray, E. J. et al., 2018, Humana Press 89-96. These signaling peptides interact with membrane receptors, allowing NPs to target the bacterial cells' surface. A cocktail of nanoparticles capable of targeting the most common bacteria causing medical-relevant biofilms can also be created. It is envisioned that a medical setting where modified NPs would be injected to the site of the infected implant, and then the patient would wear a PEMF device, resulting in a disrupted biofilm and infection. The device may also allow the magnetic nanoparticles to remain localized by continuously exerting a very mild and constant magnetic field. This would avoid dispersion of the nanoparticles systemically and would increase efficiency and efficacy. Current commercially available apparatus do not allow for administration of constant electromagnetic field in combination with the pulsed magnetic field. A well-developed animal model for implanted device-based infection is the next step to testing the utility of modified NPs and PEMF. A three-phase study is designed to verify the above disclosed hypothesis:

Phase I: In this phase, the effect of PEMF in inducing mechanical disruption of S. epidermidis and aureus is studied biofilm when applied in combination with fNPs in vitro.

Phase II: The study in Phase I is expanded to a larger animal model that allows following the regression of the infection in quasi-real-time.

Phase III: The use of a cocktail of fNPs capable of targeting the most common bacteria strains causing medical-relevant biofilms is used first in vitro and in the animal model.

### Phase I Results

A series of experiments were conducted on Staphylococcus epidermidis and aureus. Staph epidermidis cells were grown, diluted, and aliquoted onto cell culture plates. Biofilm was formed by letting the bacteria grow for 48 hrs. in the tissue culture wells. Planktonic cell growth was measured using serial dilutions and subsequently determining CFU/mL counts. Biofilm assay was performed through AlamarBlue cell viability reagent and obtaining fluorescence readings at 544/590 excitation/emission (ex/em). Magnetic nanoparticles functionalized with a lectin, Concanavalin-A, were purchase from Sigma Aldrich. Vancomycin was used as an antibiotic.

Biofilm-containing plates were exposed to different conditions and specifically:
1) No treatment
2) Vancomycin alone
3) PEMF alone
4) PEMF+Vancomycin
5) fNPs only (5 different fNPs concentration)
6) PEMF+fNPs (5 different fNPs concentration)
7) PEMF+fNPs+Vancomycin (5 different fNPs concentration)
8) NPs only (5 different fNPs concentration)
9) PEMF+NPs (5 different fNPs concentration)

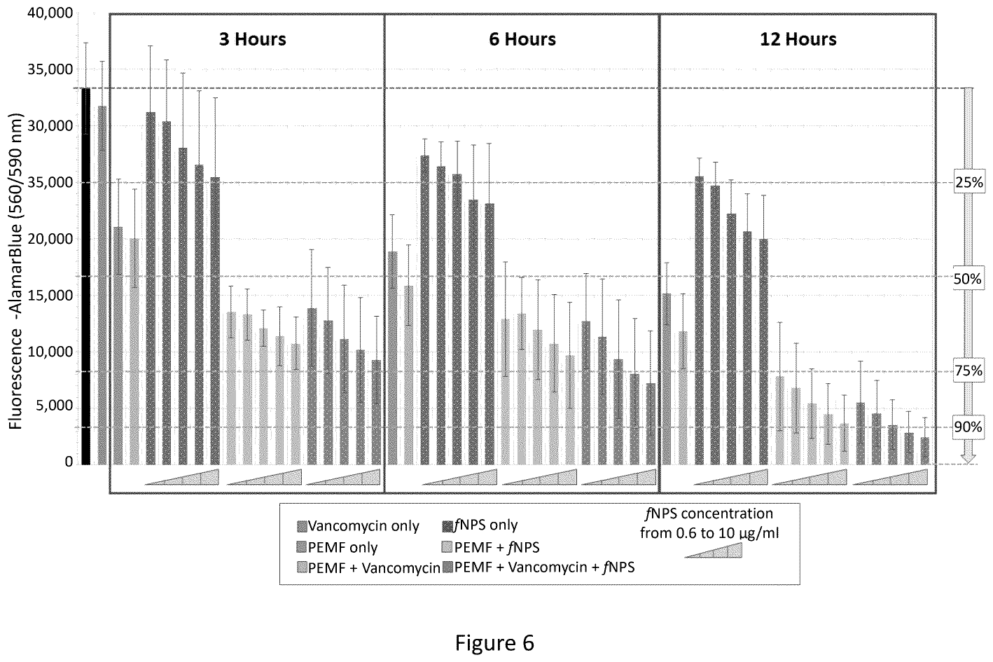

The NPs in the functionalized and non-functionalized groups had the same composition and size distribution. PEMF exposure was delivered at 37° C. and various periods: 0 hrs, 6 hrs, and 12 hrs. The results are summarized in FIG. 6.

FIG. 6 summarizes the data with functionalized nanoparticles and PEMF. In this case, the effect of fNPS+PEMF reaches levels above 90%. Note also that the addition of Vancomycin does not increase much the eradication. Also, notice how ineffective the antibiotic is on biofilm eradication (only a 5% increase) when used alone. PEMF alone instead has already an interesting effect. It is also believed that given the increase in eradication with time, by increasing the power of the PEMF signal, more than 90% eradication can be reached even at three hours exposure time. This could have implications in future clinical studies.

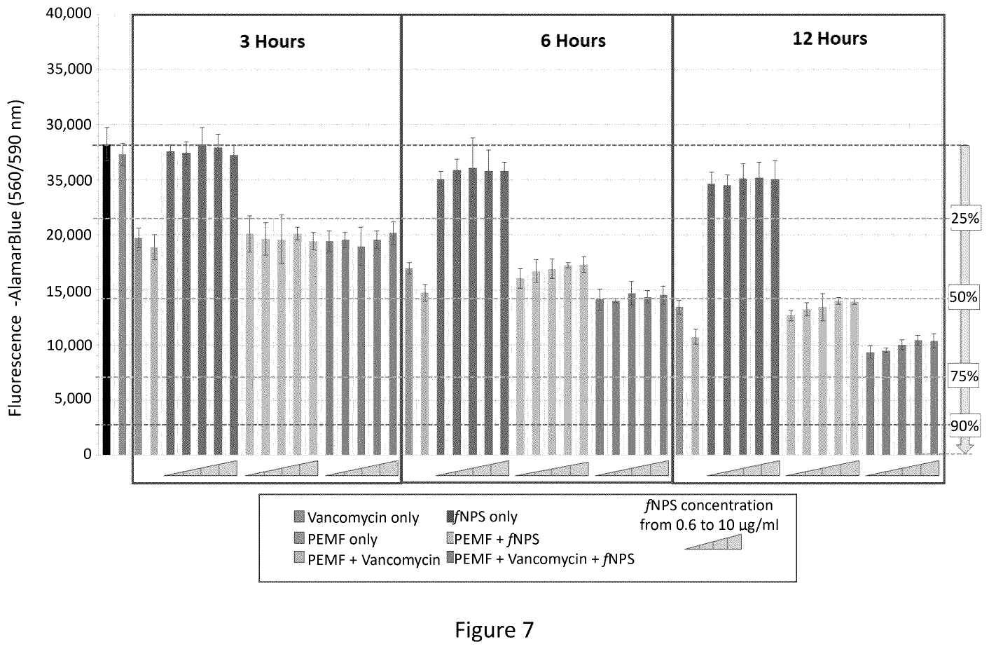

The data obtained using non-functionalized magnetic nanoparticles had the scope to demonstrate the importance of functionalization. As reported in FIG. 7, the effect of NPs+PEMF is comparable to the effect of PEMF alone, suggesting that the presence of the magnetic nanoparticles per se is not conducive to an increase in biofilm eradication.

### Example 3: A Novel Therapeutic for the Treatment of Burn Infection

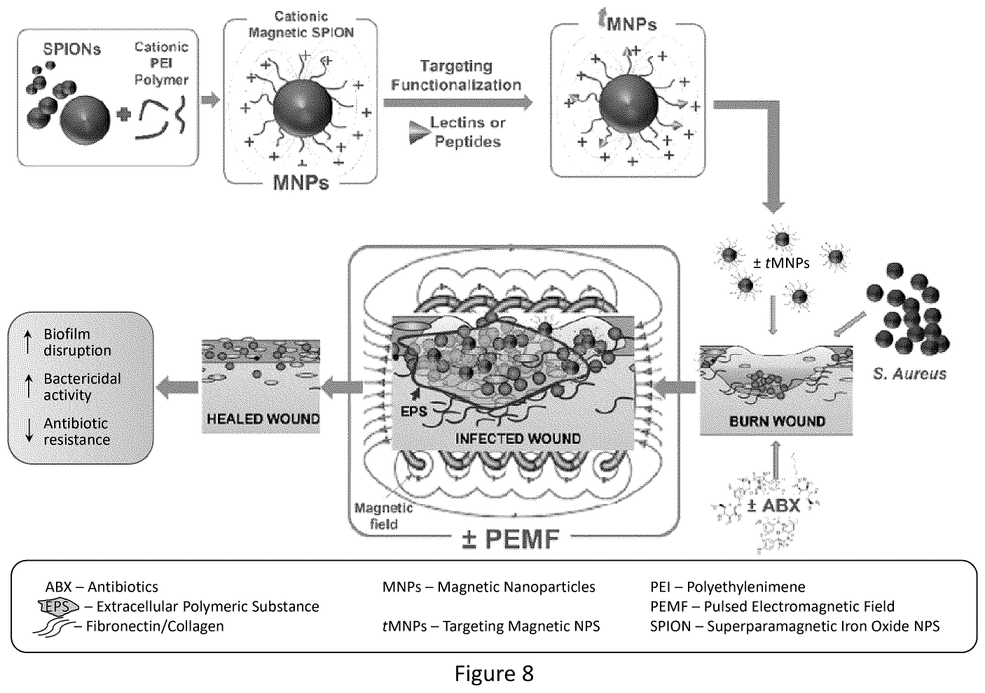

This study aims at developing a novel anti-infective therapeutic in the setting of complex combat-related burns, explicitly addressing the FY21 MBRP IDA Focus Area. It is envisioned that applications of such therapeutic at any point along the continuum of care, allows a high degree of flexibility and intervention. An external, non-contact electromagnetic stimulating device is coupled with wound dressings to deliver targeted functionalized nanoparticles for sustained antimicrobial capability and promote wound healing. A synopsis of this study is reported in FIG. 8.

### Background

Management of wartime burn casualties can be very challenging. Unlike other types of injury, burn wounds induce metabolic and inflammatory alterations that predispose the patient to various complications. In the military, burn wounds frequently occur in a polytrauma setting with other injuries related to blunt force and penetration, hemorrhaging, radiation, and blast-related injuries, in addition to other injuries which produce an additional burden to the body's innate immune response. Common to all these injuries is a high susceptibility to colonization and infection by bacteria. Hence, infection is one of the leading clinical complications associated with burn wound care and a leading cause of morbidity and mortality among burn wound patients (Branski, L. K. et al., 2009, Surg infect (larchmt) 10(5):389-397; Calum, H. et al., 2017, Curr Protoc Mouse Biol 7(2):77-87; Akers, K. S. et al., 2019, Open Forum Infect Dis, 6(s):S300-S300), with almost 61% of deaths being caused by infection (Gomez, R. et al., 2009, J Am Coll Surg 208(3):348-354). Infection is also associated with increased autograft failure and prolonged treatment regimens, recently exacerbated by the emergence of multidrug resistance (MDR) bacteria strains. Staphylococcus aureus (SA) and its methicillin-resistant variants (MRSA) are the most common pathogens infecting burn wounds among gram-positive strains (Norbury, W. et al., 2016, Surg Infect (larchmt) 17(2):250-255). Among gram-negative strains, Pseudomonas aeruginosa (PA) and Acinetobacter are the most common. The spectrum of infecting strains has recently broadened due to aggressive therapies and the survival of more severely affected patients with increasing antimicrobial resistance strains becoming predominant (Robben, P. M. et a., 2021, Surg Infect (Larchmt), 22(1):103-112). Gram-positive bacteria possess a thick (20-80 nm) cell wall as outer shell of the cell. In contrast, gram-negative bacteria have a relatively thin (<10 nm) layer of cell wall but harbor an additional outer membrane with several pores and appendices. These differences in the cell envelope confer different properties to the cell, in particular responses to external stresses, including heat, UV radiation, and antibiotics (Mai-Pronchnow, A. et al., 2016, Sci Rep 6(1):38610). Each of these bacteria also forms biofilms, surface-associated cells enclosed in an extracellular polymeric substance (EPS), which are inherently resistant to antibiotics.

A clinical need exists for a therapeutic to decrease burn wound infections with limited or no recourse to antibiotics, therefore minimizing induced antibiotic resistance.

### Preliminary Data

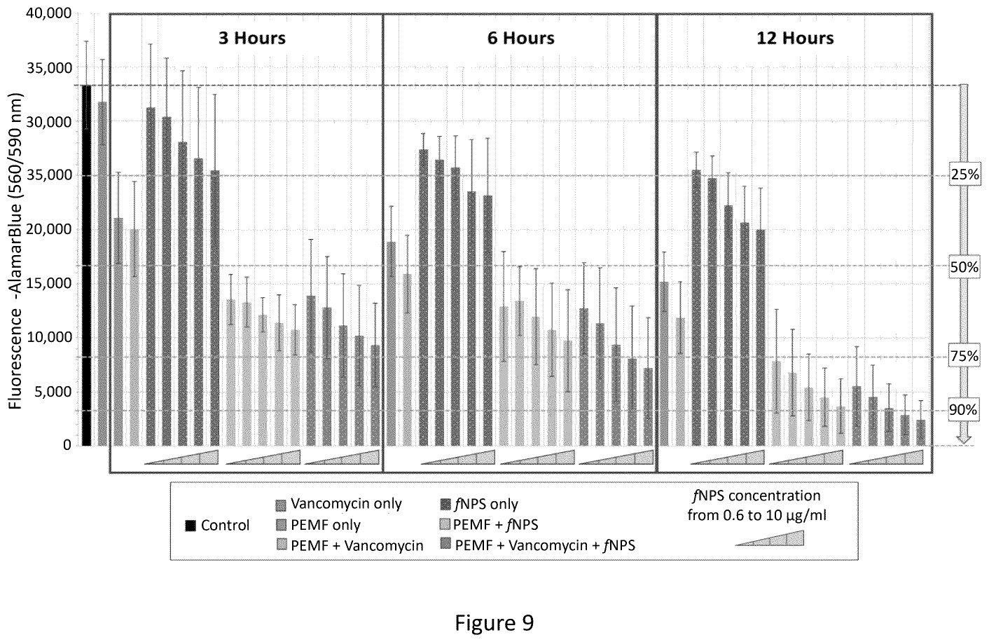

Unpublished data in the laboratory have demonstrated the efficacy of combining a pulsed electromagnetic field (PEMF) and targeted magnetic nanoparticles (t-MNPs) to increase biofilm eradication and bactericidal activity. Using Staphylococcus epidermidis (SE), the biofilm eradication increased over 80% when PEMF was administered at a frequency of 50 kHz and a magnetic field amplitude of 1.2 mT along with t-MNPs functionalized with Concanavalin-A (Con-A) that target components of the bacterial membrane, cell wall, and extracellular matrix. Importantly, the percentage of eradication only slightly increased when Vancomycin (Vanc) or Oxacillin (Ox) was added to the nanoparticles indicating that the presence of antibiotics was not required to achieve a high degree of biofilm eradication (FIG. 9).

Based on these exciting results, a novel approach is designed to treat burn wound infections and healing. Such an approach combines a non-contact external device to administer PEMF and a wound dressing as a carrier to deliver a mixture of biofilm-targeting magnetic nanoparticles functionalized with lectins or peptides and antibiotics to increase antimicrobial activity. PEMF per se has been shown to decrease inflammation, increase collagen deposition and maturation, and promote fibroblast differentiation into myofibroblast via the TGFβ pathway (Cheing, G L-Y. et al., 2014, Bioelectromagnetics 35(3):161-169; Yuan, J. et al., 2018, Cell Physiol Biochem 46(4):1581-1594), which may also contribute to a faster healing of the wound and reduced hypertrophic tissue scar formation (Moffett, J. et al., 2015, J Inflamm Res. 8:59). Moreover, PEMF has been reported to increase angiogenesis, enhance collagen deposition, and increase tensile strength in diabetic wound models (Choi, H. M. C. et al., 2018, PLoS One 13(1):e0191074). In bone tissue, PEMF has been shown to increase mineralization and collagen distribution (Benya, P. D. et al., 2021, PLoS One).

### Objective

The primary objective of this study is to extend the bactericidal efficacy of the novel application of PEMF in combination with t-MNPs to bacterial biofilms in acute burns in the skin during in vitro culture (FIG. 8). It is expected that these treatments are differentially more effective in this skin model, where the complex architecture and molecular composition of the skin and the burn tissue generates unique niches for the deposition of bacterially produced protective biofilm. Further, these treatments are more effective due to ease of administering the nanoparticles and the ability to keep them in position and keeping the desired concentration.

The second objective is to demonstrate that the above aim can be achieved with minimal or no use of antibiotics.

The efficacy of the treatment in vitro first on burn wounds with monomicrobial infections and then on polymicrobial infections using SA, MRSA, and PA is demonstrated. This work proceeds in two phases corresponding to Specific Aims 1 and 2 below.

### Hypothesis and Rationale

It was hypothesized that a) PEMF decreases biofilm formation and increases its degradation by interfering with the electrostatic attraction forces existing between the bacteria cells, their EPS, and the wound substratum, and by affecting the mobility and amount of solubilized ions; b) PEMF provokes mechanical actuation of t-MNPs that physically interact with the biofilm and disrupt it, decreasing the viability of the bacterial cells; c) t-MNPs with antimicrobial peptides (AMPs) augment the specificity or diversity of nanoparticle binding to bacteria, further increasing bacterial cell membrane damage and disruption; d) exposure to a), b), c) or their combination may dramatically increase the sensitivity of bacteria to a variety of antibiotics; e) static magnetic field superimposed on PEMF keeps the t-MNPs localized at the wound site avoiding systemic dispersion and consequent decrease in efficacy; f) PEMF facilitates cytoprotection by preventing apoptosis and reducing inflammatory cell migration, which stabilizes the local cellular environment, thus likely promoting the subsequent rapid growth of healthy new epithelium; g) PEMF in combination with t-MNPs produce a drastic reduction and containment of infections possibly decreasing the spreading and resurgence of secondary infections to adjacent tissues including bone (osteomyelitis); e) static magnetic field superimposed on PEMF keeps the t-MNPs localized at the wound site avoiding systemic dispersion and consequent decrease in efficacy; f) PEMF facilitates cytoprotection by preventing apoptosis and reducing inflammatory cell migration, which stabilizes the local cellular environment, thus likely promoting the subsequent rapid growth of healthy new epithelium; g) PEMF in combination with t-MNPs produce a drastic reduction and containment of infections possibly decreasing the spreading and resurgence of secondary infections to adjacent tissues including bone (osteomyelitis).

### Specific Aims

The above-stated hypothesis is verified and reached through the following Specific Aims:
SA-1: produce infected burn wounds in an ex-vivo porcine skin model.
1a. compare with treatments already demonstrated with biofilm on plastic.
1b. evaluate the efficacy of different PEMF/t-MNP therapies on biofilm in SA-infected wounds.
SA-2: evaluate the effectiveness of the most successful treatments from SA-1.
2a. determine effectiveness on polymicrobial infections.
2b. evaluate complete eradication of bacterial infection and the progress of wound repair.
2c. determine the efficacy of t-MNP/PEMF treatment when applied through a wound dressing.

In SA-1a, the skin model is established using short-term (4 days) high throughput assays using bioluminescent SA and the effective treatments already demonstrated with biofilm on plastic, namely PEMF, ConA-MNP, or WGA-MNP as a targeted MNP, and Vanc as a contributing antibiotic.

In SA-1b, the use of MNPs conjugated with molecules that target (bind to) different components of the biofilm or interfere with bacterial metabolism by different mechanisms is screened. Highly effective treatments are further evaluated with fluorescent bacteria and selected histologic and immunofluorescent techniques to determine bacterial and t-MNP skin penetration, viability, and resistance to treatment.

Finally, SA-2 addresses the effectiveness of the most successful treatments from SA-1 in combination with single species bacteria and polymicrobial infections. These experimental designs also extend the culture period to 8 days to determine if successfully treated cultures contain surviving bacteria that can regenerate biofilm after cessation of treatment and whether treatment enhances recovery of skin tissue biology. In addition, the efficacy of these treatments applied by wound dressing delivery is determined.

### Research Strategy

### Burn Wound Model

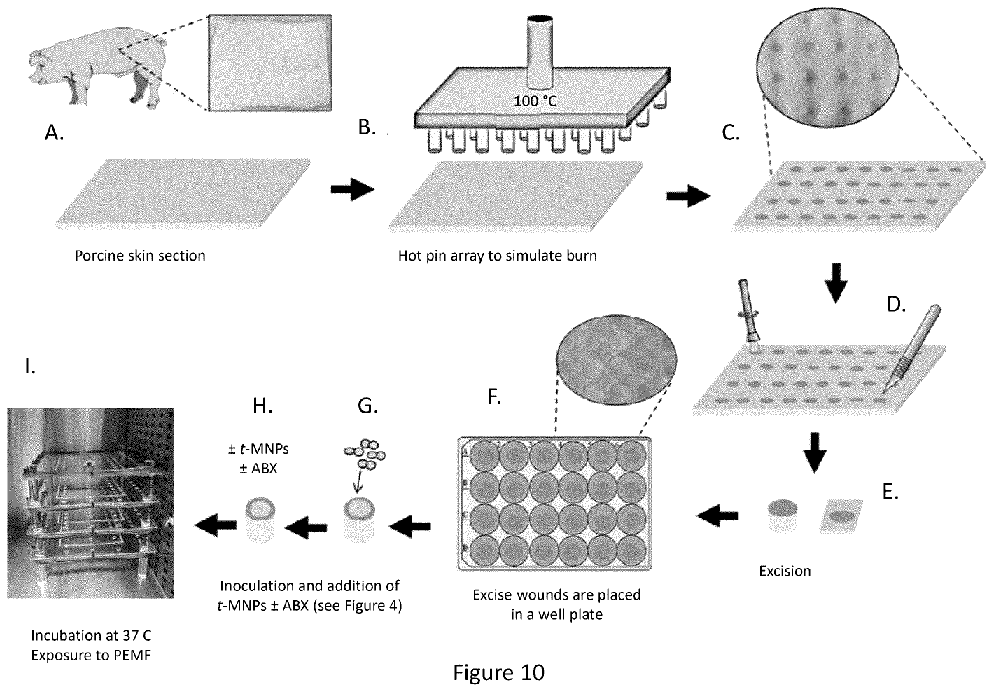

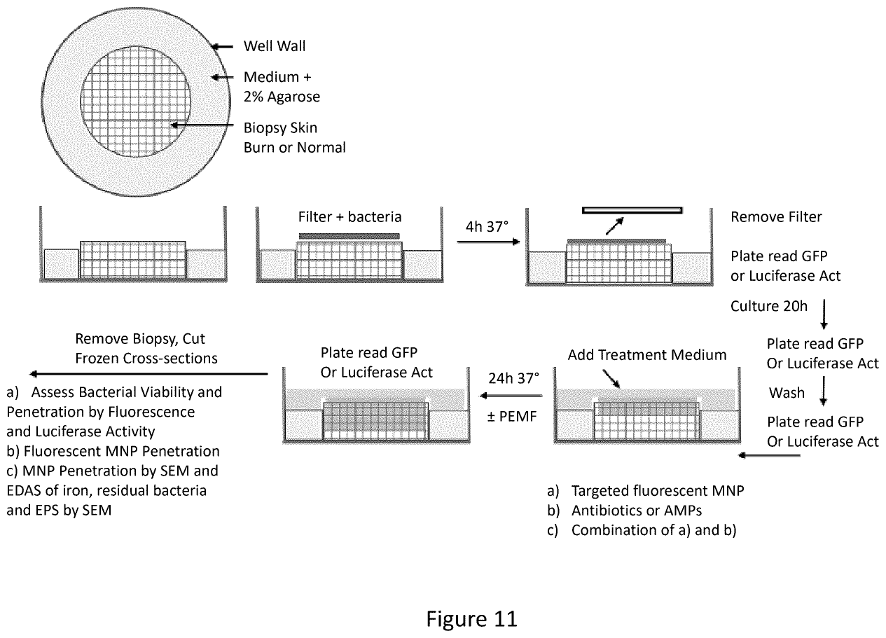

An ex-vivo porcine wound model as described elsewhere is used (Alves, D. R. et al., 2018, Front Cell Infect Microbiol 8(JUN):196). This model uses porcine skin to generate high-throughput ex vivo burns using a burn wound array device and supports studies of biofilm formation and a range of methods for monitoring bacterial growth, biofilm formation, and degradation. Burn wounds are produced with an array of twenty four 5 mm diameter brass pins that rests in a temperature-controlled heated block (FIG. 10). The device generates an array of consistent partial thickness burn injuries on the skin. Porcine skin is obtained by an authorized supplier (Sierra for Medical Science, Inc., Whittier, CA). Areas of the skin are initially prepared by shaving and surface disinfecting in 70% ethanol solution. After production, individual wounds are excised using a punch biopsy and transferred into the wells of tissue culture plates. Individual wounds are inoculated with test organisms to cause infection and wound biofilm formation. A schematic of inoculation, the culture protocol, treatments, and outcome measures is presented in FIG. 11.

Preparation of t-MNPs

The approach reported by Wang et al. (Wang, X. et al., 2018, J Mater Sci 53(9):6433-6449) was modified so that fluorescence can be incorporated into MNPs before the attachment of the targeting molecule. Thus, it is not modified by labeling, which is especially important for LL-37 and Vanc. Briefly, iron-oxide nanoparticles are modified sequentially with citrate, coated with deacylated chitosan/acrylic acid, reacted with 1/10 molar ratio of Alexa Fluor-NHS in appropriate colors, and coated with PEG dicarboxylic acid. Targeting molecules (WGA and Vanc) are then linked through their amines to the carboxyls using EDC and NHS (Xie, H-Y et al., 2007, Bioconjug Chem 18(6):1749-1755), except for LL-37. LL-37 undergoes self-assembly in a membrane or membrane-like environment to form amphipathic, alpha-helical antiparallel dimers and tetramers, the latter capable of forming transmembrane channels that contribute to its antibacterial effects (Sancho-Vaello, E. et al., 2020, Sci Rep 10(1):17356). Peptide-peptide interactions between the interior cores of the peptides are responsible for this essential behavior (Sancho-Vaello, E. et al., 2020, Sci Rep 10(1):17356). LL-37 (purchased from Peptide 2.0 (www.peptide20.com)) is synthesized with an N-terminal sulfhydryl-reactive maleimide so that LL-37 can be coupled to the carboxyl MNPs functionalized with 2-aminoethanethiol. This results in an oriented display without compromising the peptide core amines/residues needed for dimer and tetramer interaction or channel formation. Conjugation with MNP is performed at 6 and 50 g/ml peptide concentrations, the latter allowing preassembly of multimers (Gao, Y. et al., 2020, ACS Appl Bio Mater 3(11):7696-7705). Such LL-37-MNPs are tested in the standard skin model at different concentrations alone or in the presence of 0.2-6 g/ml soluble LL-37, the latter to enable in situ assembly of dimers and tetramers on the MNP while still allowing transduction of the PEMF signal. Coupling efficiency for WGA and LL-37 is determined by the difference between the soluble concentrations at the beginning and the end of the reaction. For Vanc, the fluorescent target Fluorescein-Lys-D-Ala-D-Ala is used to measure immobilized functional Vanc (Hassan M.M. et al., 2017, Bioconjug Chem 28(2):353-361). t-MNPs size distribution is characterized via dynamic laser scatter diffraction (DLS) and Scanning Electron Microscopy (SEM) (Gibson, A. L. F. et al., 2017, J Cutan Pathol 44(12):998-1004). The process is started with 60-200 nm t-MNPs, and the size is adjusted based on initial results. The choice of targeting molecule serves to increase bacterial specificity or broaden reactivity, both to extend efficacy.

### Pemf Conditions

PEMF treatments characterized by a magnetic field amplitude from 0.5 to 2.0 mT and at least two different pulse frequencies (e.g., 5 kHz and 50 kHz), and a triangular waveform at different exposure times (e.g., 4, 12, and 24 hours) are used, utilizing a custom PEMF device already in use in the lab (FIG. 10).

### Outcome Measures

Live bacterial content is measured by bioluminescence in a BMG Omega plate reader using a top optic with well scanning and the total intensity recorded. This is the primary determinant of bacterial killing/survival associated with each treatment. Each biopsy is then fixed, divided in half and half processed for paraffin sectioning, and the other half embedded in OCT for frozen sections. Paraffin sections is stained by H&E, gram stain, EVG (Elastin Von Gieson) for collagen architecture/thermal denaturation, and fluorescence for MNP penetration. Frozen sections are stained for LDH activity for live cells, procollagen-C-terminus antibodies for live fibroblasts, fluorescent MNP, and Ki-67 as a proliferation marker. To validate the results, the experiments are repeated using GFP-expressing SA (strain RN4220 containing the green fluorescent protein (GFP) reporter plasmid pSGFPS1, NR-51163, from BEI Resources/NIAID) and the distribution of living bacteria on paraffin and frozen sections using a fixation insensitive goat antibody to GFP (Abcam, ab6673).

### Specific Aim 1

To produce infected burn wounds in an ex vivo porcine skin model. SA-1a. To compare with treatments already demonstrated with biofilm on plastic.

### Rationale

The primary goal is to optimize the in vitro skin burn wound model with bacterial infection for investigation of the bactericidal benefits of the application of PEMF with targeted MNPs. The intent in this aim is to focus predominantly on the viability of the infecting bacteria during and after a variety of treatments shown to enhance bacterial killing in the preliminary study with bacteria on plastic substrates. In this wound model, the bacterial environment is much more complex and conducive to the deposition of biofilm that serves as a protective envelop in the face of a variety of antibiotic treatments.

### Approach

The standard model is derived from Alves et al. (Alves, D. R. et al., 2018, Front Cell Infect Microbiol, 8(June)) and is performed in 24-well plates as described in the diagram (FIG. 11). Twelve experimental conditions are tested in two plates derived from adjacent skin biopsies: Control skin (CS), Burn skin (BS), CS+infection, BS+Infection, BS+the treatments specified in Table 1.

The bacteria is bioluminescent SA Xen36 from Perkin Elmer. Wounding is performed using a 24-pin array (5 mm diameter pins) heated to a consistent temperature of 100° C. (Liu, A. et al., 2021, Burns 47(3):611-620) with exposure for 5, 15, and 30 sec to identify a deep partial thickness wound assessed by viability with LDH and EVG for collagen damage on frozen sections (Gibson, A. L. F. et al., J Cutan Pathol 44(12):998-1004). Cell death to only 80% depth is chosen so that necrotic tissue does not provide a direct path through the complete skin sample. 7 mm biopsies is taken concentric with the burn sites allowing a 1 mm rim of normal skin around each burn site biopsy and established in the wells surrounded but not covered by 2% LGT agarose in Dulbecco's Modified Eagle's Medium/Ham's F12 (3:1), 2% fetal calf serum+supplements (Coolen, N. A. et al., 2008, Wound Repair Regen 16(4):559-567). In a subset of experiments focused on histology and fluorescence/immunofluorescence, the biopsies are shifted relative to the burn site so that 50% of the biopsy is burned tissue with immediately adjacent 50% non-burn tissue so that burn-dependent gradients of response can be followed. Cultures are for four hours with filter stabilized bacteria on the top of the biopsies to establish and localize the infection, followed by filter removal and 20h of culture to provide bacterial proliferation and biofilm formation. This is followed by a 24h treatment period and washing to remove loosely adherent planktonic bacteria. These experiments are repeated using different PEMF conditions, antibiotic (Vanc) concentrations, and t-MNP concentrations/sizes to establish the most instructive experimental format. It is apparent from FIG. 9 that the t-MNP effects are both dose and duration-dependent; thus, the extension of these parameters are evaluated.

TABLE 1
Treatments

a. Antibiotics only
b. PEMF only
c. PEMF + Antibiotic
d. PEMF + MNP
e. PEMF + MNP + Antibiotic
f. PEMF + t-MNP
g. PEMF + t-MNP + Antibiotic

### Anticipated Results

It is expected that the results are supportive and extend the beneficial effects of PEMF and PEMF/t-MNP on bacterial cell death previously obtained on plastic. In particular on PEMF alone and in combination with t-MNP. Indeed, PEMF penetrates tissue well while antibiotics less so and are subject to hindrance due to biofilm. The effects of PEMF alone are due to its interaction with bacteria intrinsic targets in the same way that PEMF stimulates some mammalian cells through L-type voltage-gated ion channels and A2A and A3 adenosine receptors (Petecchia, L. et al., 2015, Sci Rep 5(1):13856; Mediero, A. et al., 2015, FASEB J. 29(4):1577-1590; Vincenzi, F. et al., 2013, PLoS One, 8(5):e65561). Thus, PEMF appears to engage new bactericidal pathways.

Thus, increasing the power, altering the frequency, and the duration of PEMF may provide effects deeper in the wound than the conditions used on biofilm on plastic and decrease the need or concentration of synergizing antibiotics. Learning the penetration of WGA-MNP in the skin wound allows optimizing the MNP size to gain better penetration with and without PEMF.

### Potential Pitfalls and Alternative Approaches

If the t-MNPs do not penetrate the biofilm-filled wound to the bottom of the biofilm, its benefit are not uniformly realized. Making smaller t-MNP is a planned solution for this that supplements another useful alternative. The application of an intermittent or superimposed directional magnetic field to drive the t-MNP deeper in the wound supplement the increase in diffusion caused by PEMF and the transduction of its effects through the t-MNP to the bacteria.

SA-1b. To evaluate the efficacy of different therapies on biofilm reduction in SA-infected wounds.

### Rationale

SA-1a establishes the validity of the burn wound model and its application with testing PEMF/t-MNP bactericidal effects using the pair of t-MNP (Wheat Germ Agglutinin, WGA) and antibiotic (Vanc). The goal of SA1b is to screen alternative pairs for synergistic bacterial killing.

### Rationale for Choice of Targeting Molecules

### Overall Strategy Exemplified by Vanc

Hassan et al. (Hassan, M. M. et al., 2017, Bioconjug Chem, 28(2):353-361) have clearly demonstrated that high local density of immobilized Vanc arrayed on the surface of 200 nm MNP dramatically reduced the MIC (10-100-fold) relative to soluble Vanc. This is likely due to a fixed closer location between cell wall binding sites. The increased effectiveness was also capable of converting a Vanc-resistant strain to Vanc-sensitive. It was suggested that the benefits of immobilization and close proximity membrane interactions might be a generalizable paradigm for other conventional antibiotics. This concept is used with the MNP targeting molecules and expect even further enhancements when these MNPs are driven by PEMF. In the case of Vanc, its increased D-Ala-D-Ala binding-dependent blockage of peptidoglycan synthesis and crosslinking in the cell wall may also enhance the intracellular availability/effectiveness of other tested antibiotics.

### LL-37

The human cathelicidin LL-37 is an antimicrobial peptide (AMP) that binds to both gram-positive and gram-negative bacteria's outer, inner, and transmembrane domains to disrupt the membrane structure and eventually generate transmembrane peptide-dependent channels that depolarize the membrane and lead to bacterial death (16). LL-37 is used both as an antibiotic and as means to target MNPs to membrane components to transduce the effects of PEMF, alone or in the presence of other conventional antibiotics that may synergize with LL-37 (Kim, E. Y. et al., 2017, Eur J Med Chem, 136:428-441; Zhou, Y. et al., 2013, Exp Ther Med, 6(4):1000-1004; Xia, Y. et al., 2021, PLOS Pathog, 17(9):e1009909; Shurko, J. F. et al., 2018, J Antibiot (Tokyo), 71(11):971-974; Le, J. et al., 2016, Eur J Clin Microbiol Infect Dis, 35(9):1441-1447; Dosler, S. et al., 2014, Peptides, 62:32-37; Ridyard, K. E. et al., 2021, Antibiotics, 10(6):650) and lead to their greater availability/effectiveness at target sites. These two approaches are also expected to be synergistic since binding of LL-37 conjugated to MNP and in the presence of PEMF may dramatically increase the integration of LL-37 and its disruption of membranes in addition to increasing the penetration of the coupled LL-37 through the biofilm. Increased penetration and killing by LL-37 MNP is also assessed in the presence of WGA-MNP and PEMF for its capacity to recognize and affect biofilm polysaccharide intercellular adhesin (PIA) and peptidoglycan. Enhancement of LL-37 antibiotic capacity by its oriented high-density immobilization on MNP and its capacity to synergize with other antibiotics, especially under the influence of PEMF is expected.

LL-37 provides additional advantages for application to skin burn wounds. In addition to its antimicrobial effects, it is also anti-biofilm and anti-adhesion (Dean, S. N. et al., 2011, BMC Microbiol, 11(1):114). It also suppresses the proinflammatory response of fibroblasts and macrophages by inhibiting the production of nitrite, IL6, TNF-α, and MCP-1.34 LL-37 stimulates wound closure by enhancing re-epithelization, angiogenesis, and fibroblast proliferation (Dean, S. N. et al., 2011, BMC Microbiol, 11(1):114; Carretero, M. et al., 2008, J Invest Dermatol, 128(1):223-236). It exhibits an acceptable cellular specificity profile being non-toxic to mammalian cells at effective antimicrobial concentrations, and this should improve with PEMF/t-MNP synergy. Stability to proteolysis in the in vivo environment can be enhanced by partially or completely substituting D-amino acids for L-amino acids (Ridyard, K. E. et al., 2021, Antibiotics, 10(6):650; Dean, S. N. et al., 2011, BMC Microbiol, 11(1):114; Kim, E. Y. et al., 2017, Eur J Med Chem, 136:428-441). Although there is adequate evidence that some bacteria can become resistant to AMPs (Ridyard, K. E. et al., 2021, Antibiotics, 10(6):650), the use of LL-37 and conventional antibiotics in synergistic combinations can block this resistance (Ridyard, K. E. et al., 2021, Antibiotics, 10(6):650).

### WGA

This carbohydrate-binding lectin possesses a low intrinsic antibacterial character. WGA binds N-acetyl glucosamine oligosaccharides on peptidoglycan of the bacterial cell wall (Biari, K. et al., 2019, Nat Prod Commun, 14(5):1934578X1984924) and extracellular Polysaccharide Intercellular Adhesin (PIA) (Formosa-Dague, C. et al., 2016, ACS Nano, 10(3):3443-3452), a polysaccharide of the extracellular matrix or EPS. Wheat Germ Agglutinin (WGA) bound to MNP serves to transduce the effects of PEMF and lead to disruption of the EPS and/or cell wall and facilitation of transport of soluble or MNP bound antibiotics to their target sites.

### Approach

SA-1b is performed in a two-phase manner, first by truncating the standard experimental design from SA-1a to just include the assays up to the bioluminescence measurements to determine the degree of bacterial killing by each of the potential synergistic pairs. Secondly, based on these results, promising pairs are evaluated in the extended detail of SA-1 because t-MNP concentration, antibiotic concentration, and PEMF characteristics may differ from those determined in SA-1. A table of pairs is shown in Table 2. It is already known that some of these pairs have shown synergy in the literature, but it is the first time to see if these or other pairs are more synergistic in the presence of targeted MNPs and the driver PEMF. In addition, pairs of t-MNPs are also tested (i.e., WGA-MNP/LL-37-MNP; WGA-MNP/Vanc-MNP; LL-37-MNP/Vanc-MNP) because the effects of both targeting molecules in the pair may be increased by this presentation and make it more difficult for the bacteria to survive the coupled damage. Initial experiments are performed with antibiotic concentrations of 0.2, 1, and 5×MIC of the soluble form so that there is assay space to detect enhancement and also detect no response. Reevaluation depends on these results. Large potential enhancements with PEMF and t-MNPs are possible, and as a part of the experimental variables (see Table 1), it is also determined if a particular t-MNP in the presence of PEMF yields complete bacterial killing alone.

TABLE 2
Pairs indicated by ± mean that only the t-MNP is tested so that the
same soluble molecule is not competing for binding with the t-MNP. See
Preparation of t-MNPs for exception with LL-37-MNP. All antibiotics are
purchased from Sigma Aldrich. Each targets a different bacterial process.

| | | MNP Targeting Molecule → | | |
|---|---|---|---|---|
| | | WGA | LL-37 | Vanc |
| | | Bacterial Target → | | |
| | | | Membrane surface, | Division |
| | | Glucosamine EPS/matrix | transmembrane | septum of |
| | | Peptidoglycan/Cell wall | channel | cell wall |
| Synergizing | Vancomycin | + | + | ± |
| Antibiotic | LL-37 | + | ± | + |
| | Rifampicin | + | + | + |
| | Trimethoprim | + | + | + |
| | Penicillin | + | + | + |

### Anticipated Results

The relative bactericidal effectiveness of WGA-MNP, LL-37-MNP, and Vanc-MNP in transducing the effects of PEMF to the bacteria in the biofilm burn wound alone and in the presence of 4 different antibiotics is determined. Each MNP has a different targeting profile, and it is expected that one or more result in extensive bacterial death, alone or in combination with another antibiotic. It is expected that the combinations of t-MNP and antibiotics is superior to the use of antibiotics alone.

### Potential Pitfalls and Alternative Approaches

A potential difficulty is that simply replicating the conditions derived in SA-1a with the array of treatments to be tested in this Specific Aim only provides a starting point for optimization. Conditions of PEMF and concentrations of t-MNP may be different for each t-MNP and antibiotic combination because the mechanism of the bacterial target disruption is different, effects downstream of the PEMF target may also be sensitive to PEMF, and the antibiotics exhibit different mechanisms. This has been taken into account by efficiently screening treatment conditions initially with the straightforward bioluminescence assay of bacterial survival.

### Specific Aim 2

To evaluate the effectiveness of the most successful treatments from SA-1. The primary goals are a) to determine whether successful pairs from SA-1b or modifications of these pairs are successful in treating polymicrobial infections in this skin model (these are most likely in the clinic). b) To determine if any identified treatment is entirely successful in eradicating the bacterial infection and stimulates wound repair. c) Determine the efficacy of t-MNP/PEMF treatment when applied through a wound dressing.

SA-2a. To determine effectiveness on polymicrobial infections.

### Rationale

Although the prevalent strain infecting burn wound is SA, the polytrauma nature of combat burn wounds make them susceptible to colonization from different bacteria strains. In particular, it is not uncommon for gram-negative strains to be present, the most prevalent being PA. Therefore, the effect of the novel therapeutic on the presence of composite SA, MRSA, and PA biofilms is tested.

### Approach

A polymicrobial three-part infection with bioluminescent SA (IVISbrite Staphylococcus aureus Xen36, Perkin Elmer), MRSA (IVISbrite™ Staphylococcus aureus Xen31), and PA (IVISbrite™ Pseudomonas aeruginosa Xen41) as individual bacteria or a mixture is evaluated in the same two-phase experimental strategy presented in SA-1b using successful synergistic pairs and the t-MNP antibiotics LL-37 and Vanc alone. Wound penetration analysis use MRSA (MW2)-GFP38 and PA-GFP (ATCC 10145GFP). In addition, the combination of three simultaneously immobilized AMPs (HNP-1, hBD-1, and LL-37) are tested which has been shown to completely eradicate similar polymicrobial infections and demonstrated the advantage of utilizing peptide anchorage to increase bacterial membrane stress for killing (Gao, Y. et al., 2020, ACS Appl Bio Mater, 3(11):7696-7705). All three are mixed and immobilized to make a composite t-MNP that is tested alone or in combination with a synergistic antibiotic, as selected in SA-1b.

### Anticipated Results

It is expected that the combination of t-MNP/PEMF alone or in combination with a conventional antibiotic is effective against the proposed polymicrobial infection. The most likely successful treatments is LL-37-MNP alone or in combination because of its known activity against both gram-positive and gram-negative bacteria and its membrane disruption mechanism of action, which is expected to facilitate target access to other antibiotics.

### Potential Pitfalls and Alternative Approaches

A limitation is that vancomycin is ineffective against gram-negative bacteria because it does not penetrate the outer cell membrane to access its target peptidoglycan. By pairing in the presence of PEMF with LL-37-MNP (and perhaps WGA-MNP), membrane disruption may permit at least soluble vancomycin (perhaps Vanc-MNP) to gain access to its Lys-D-Ala-D-Ala peptidoglycan target in gram-negative as well as gram-positive bacteria cell walls to cause cell death. Moreover, the possibility of using multiple soluble antibiotics for a polymicrobial infection and/or using mix of functional nanoparticles would undoubtedly broaden the effectiveness of these combinations in a more diverse polymicrobial infection.

### SA-2b

To evaluate eradication of bacterial infection and the progress of wound repair.

### Rationale

For evaluation of eradication, the culture period is extended so that treated biopsies can be subsequently cultured in the absence of treatment to allow the growth of any residual bacteria detected by bioluminescence. This is a stringent test of the treatment effectiveness and allows extended or modified treatments to be tested for complete eradication. This extended culture is also the best opportunity to evaluate whether treatment facilitates/modifies the repair process compared to uninfected burn wounds; benefits of treatment to repair in addition to bacterial killing strengthen the case for clinical application.

### Approach

The groups experience with constructing, culturing, and transplanting human skin equivalents to nude mice (Lee, Y-S et al., 2016, Wound Repair Regen, 24(2):302-316) is advantageous to extend the duration of the burn wound model in culture to follow the completeness of bacterial killing when uniform medium-based delivery of t-MNPs is compared to topical wound dressing delivery. Markers of tissue repair in these two situations are also evaluated. In these 8-day cultures, the biopsies are supported on two layers of coarse nylon mesh to facilitate feeding from below and enable culture at the air/liquid interface. No surrounding agarose are present so that medium changes can occur every other day, and the medium only contact the biopsy at its edges. Medium is Dulbecco's Modified Eagle's Medium/Ham's F12 (3:1), 2% fetal calf serum supplemented with 1 M hydrocortisone, 1 μM isoproterenol, 0.1 M insulin, 1.0×10−5 M L-carnitine, 1.0 ×10−2 M L-serine, 1 M DL-α-tocopherol, 130 g/mL ascorbic acid, and a lipid supplement (containing 25 μM palmitic acid, 15 M linoleic acid, 7 M arachidonic acid, and 24 M bovine serum albumin) (Coolen, N. A. et al., 2008, Wound Repair Regen, 16(4):559-567). The infection is done as above and treatment begins at 24 h and continue for 24h before bioluminescence measurement, washing and measurement again. Immediately following this, the biopsies are transferred to a new 24 well plate. Bioluminescence is measured prior to every subsequent feeding, either without or with continued treatment. Without treatment, it is determined if the biopsy can be recolonized by a small number of protected bacteria. If no bacteria reappeared, this would indicate that the treatment was capable of killing bacteria regardless of their skin/biofilm environment. Alternatively, the infection period is increased from 24 to 48h, before starting treatment for 24 and 48h, to determine if treatment could eradicate a more established biofilm, a situation often encountered in the clinic. As part of these longer studies, it is determined if particular treatment protocols facilitate wound repair. Immunofluorescent imaging for increased proliferation using expression of Ki-67 (Anti-Ki67 antibody (ab15580), Abcam) is coupled to markers of fibroblasts (Anti-Procollagen type I C-peptide (MO 11) TaKaRa) and keratinocytes (Anti-Cytokeratin 17 (ab238808), Abcam) (Coolen, N. A. et al., 2008, Wound Repair Regen, 16(4):559-567). Recovering cell viability is determined with LDH (Gibson, A. L. F. et al., 2017, J Cutan Pathol, 44(12):998-1004). In addition, progress toward wound closure is measured by the degree of re-epithelialization with H&E staining. In each case, the biopsies is displaced by 50% relative to the burn site so that the interface between normal and burn tissue can be evaluated using these markers. This arm of the experimental design is warranted because both PEMF (Cheing, GL-Y. et al., 2014, Bioelectromagnetics, 35(3):161-169; Yuan, J. et al., 2018, Cell Physiol Biochem, 46(4):1581-1594; Moffett, J. et al., 2015, J Inflamm Res, 8:59; Choi, H.M.C. e al., 2018, PLoS One, 13(1):e0191074), and LL-37 (Dean, S. N. et al., 2011, BMC Microbiol, 11(1):114; Carretero, M. et al., 2008, J Invest Dermatol, 128(1):223-236; Song, D. W. et al., 2016, Acta Biomater, 39:146-155; Duplantier, A. J. et al., 2013, Front Immunol, 4(JUL):143) exhibit independent stimulation of wound repair.

### Anticipated Results

A stringent test of complete bacterial eradication is developed in the presence of complex wound architecture and protective biofilm formation. This is important in the clinical situation so that recurrent infection does not occur or spread. The 8-day culture is expected to provide adequate treatment time and subsequent treatment-free culture to expose any protected bacteria to detection by sensitive bioluminescence assay. If none reappear, that provides a strong support for clinical evaluation of the tested treatment.

### Potential Pitfalls and Alternative Approaches

Two potential pitfalls are present. The first is the viability of the skin biopsies over the 8-day culture period. Important changes (lifting to the air/liquid interface, frequent medium changes, and appropriate supplementation of the culture medium) are made that have led to even longer time (21 days) culture success of similar biopsies (Coolen, N. A. et al., 2008, Wound Repair Regen, 16(4):559-567). The second potential difficulty is the inadvertent contamination of the treated and then treatment (antibiotic)-free cultures. This is addressed by transfer to new culture plates at each feeding and thorough biopsy washing before feeding and transfer.

SA-2c—To determine the efficacy of t-MNP/PEMF treatment when applied through a wound dressing.

### Rationale

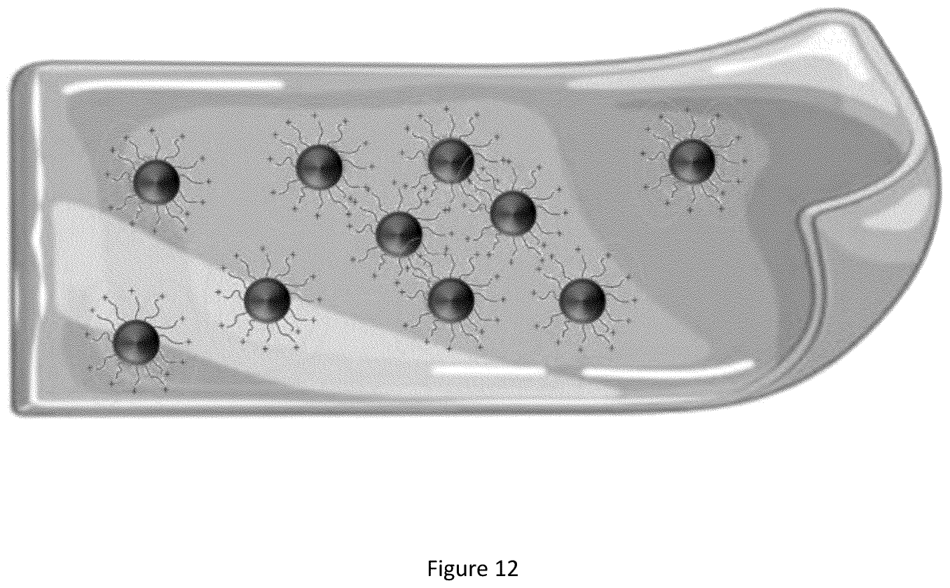

A wound dressing that can deliver t-MNP (FIG. 12) is significant to clinical application of the t-MNP/PEMF treatment efficiency. Moreover, systemically administered antibiotics can encounter difficulties reaching damaged skin tissue due to compromised blood circulation, making them ineffective for reducing bacterial counts in granulation wounds. Therefore, should the use of antibiotics be required, their dispersion inside the wound dressing allows direct contact to the burn site.

### Approach

Thet et al. (Thet, N. T. et al., 2016, ACS Appl Mater Interfaces, 8924):14909-14919) have utilized a 2% agarose hydrogel dressing to localize a bacteria-sensitive detection reagent for early detection and localization of skin infections. The 2% agarose was patterned with an array of wells that contained 0.7% agarose and the detection reagent. This approach is tested with t-MNP contained in the 0.7% agarose wells to allow more rapid access to the underlying burn/control biopsy while maintaining the structural integrity of the dressing with the 2% agarose. Sheets of dressing (28×28 mm) is cast with 3D-printed molds and 2% agarose in culture medium to be 3 mm thick with arrayed 1 mm square×1.5 mm deep wells and 0.7 mm thick well walls. After filling wells with 0.7% agarose containing t-MNP alone or with antibiotic, 7 mm punch biopsy needles is used to remove dressing circles to match the biopsies of the model. These are inverted to place the gelled well openings against the wound and the stabilizing 2% agarose on the top. This also serve to localize the t-MNP delivery under the condition of culture at the air/liquid interface. The dressing is removed or replaced every other day to follow complete eradication or determine the effects of continuing treatment, respectively. It is important to note that application of polarized intermittent or superimposed magnetic fields along with PEMF function to aid the delivery of t-MNPs from the localized dressing to and throughout the wound site.

Consequently, the wound distribution of the fluorescent t-MNPs is carefully monitored in addition to their co-distribution with any remaining fluorescent bacteria. In addition, modified agarose may allow higher concentrations of t-MNPs to be held and slowly released between dressing changes. Any successful dressing/treatment combination is also tested using a loaded dressing that has been lyophilized, useful for storage and field application. The proposed agarose dressing, or a layered composite dressing may also serve to decrease wound desiccation.

### Anticipated Results

t-MNPs is delivered at the burn site with increased efficiency and efficacy. The transparency of the agarose gel allows monitoring bacterial growth by bioluminescence directly. The presence of this dressing may also enhance the limited wound repair possible in this model, reflecting potential clinical benefits.

### Potential Pitfalls and Alternative Approaches

Should the agarose gel not work as anticipated, the use of other gels as carriers for the t-MNPs, for example, polyethylene glycol (PEGylated) fibrin gels or chitosan-based hydrogels is explored (Stoica, A. E. et al., 2020, Materials (Basel), 13(12):2853; Huang, W. et al., 2018, ACS Appl Mater Interfaces, 10(48):41076-41088; Seetheraman, S. et al., 2011, Acta Biomater, 7(7):2787-2796).

### Statistics

Statistics is performed using 1-tailed unpaired student's t-test and ANOVA for all the experiments, with p<0.05 considered significant. Data for multiple comparisons are analyzed using a one-way ANOVA (analysis of variance) followed by the Tukey test. All data are expressed as the mean and the standard error of the mean (SEM), where indicated. Values of p<0.05 is considered significant.

### Innovation

There are several innovative aspects in this study. The use of biofilm-targeting magnetic nanoparticles and electromagnetic fields are combined to create a novel approach mediated by multiple sites of intervention to prevent or decrease the bacterial protective effects of biofilm and produce bacterial killing. These effects can occur in the absence of antibiotics, minimizing the opportunity for antibiotic resistance, but may also substantially increase the efficacy of antibiotics. PEMF is applied with a non-contact device, beneficial with fragile burn wounds, and PEMF also enhances wound healing. The increased efficacy of PEMF/t-MNPs relative to antibiotics alone likely protect against the infection spreading to surrounding tissues, including bone. PEMF may also reduce scar tissue formation and have a synergistic effect with scar-reducing agents. The variety of nanoparticle targeting offer the additional benefits of targeting individual components of polymicrobial infections and increasing bactericidal effects by treating with a cocktail of differently targeted MNPs. Moreover, this therapy is employed in several different settings along the continuum of care, allowing a high degree of flexibility. PEMF/t-MNPs use is unique to this laboratory and generates an innovative platform for addressing the clinical need to prevent or correct infection in burn wounds.

### Impact

In the proposed treatment strategies, the absence or a much reduced concentration of conventional antibiotics and their only local application greatly reduce the possibility of developed resistance. The utilization of PEMF and t-MNP is expected to generate rapid treatment penetration of burn wounds and rapid bacterial killing even in cases of established biofilm produced by both gram-positive and gram-negative bacteria. Thus, the application of t-MNP/PEMF may greatly broaden the bactericidal spectrum and effectiveness of conventional antibiotics and lead to re-initiation of their use. These treatment assets are expected to remove protected bacteria and prevent long-term recurrence of local or systemic infection.

### Example 4: Electro Magnetic Acoustic Transducer Network in the Enhancement of Anti-Infective Strategies

A system utilizing pulsed electromagnetic fields (PEMF) was engineered to treat biofilm-based orthopedic implant and surgical site infections. The feasibility of using such a system as a non-invasive and safe technology for preventing or eradicating such infections was demonstrated. The hypothesis was that PEMF exhibits anti-biofilm properties by disrupting the attractive electrostatic interactions that work to keep the biofilm structure intact and keep the cells adhered to a physiological surface in the body or on an implant. PEMF also induces several additional anti-biofilm effects that could also enhance the efficacy of antibiotic and other anti-biofilm and antimicrobial therapies.

The primary goals accomplished were to optimize the anti-biofilm effectiveness of the PEMF system and determine if PEMFs biofilm eradication ability would be further useful in enhancing the effectiveness of antibiotic treatments. Through initial experiments using the PEMF system, it was determined that a pulsed EMF wave at a fundamental (burst) frequency of 15 Hz applied in high physiological frequency, at 40 kHz pulsed frequency and 1.2 mT magnetic field amplitude was advantageous over a low pulsed frequency.

Initial studies concerned the design of the EMATs network. The initial goal was to demonstrate the uniform distribution of both the acoustic waves and the magnetic field on the metal surfaces. To this extent, a computer model of the EMAT was constructed and a simulation study run a necessary step in the evaluation of the combined effect of the acousto-magnetic excitation and to obtain data in support of the controllable exposure of the bacteria by EMAT.

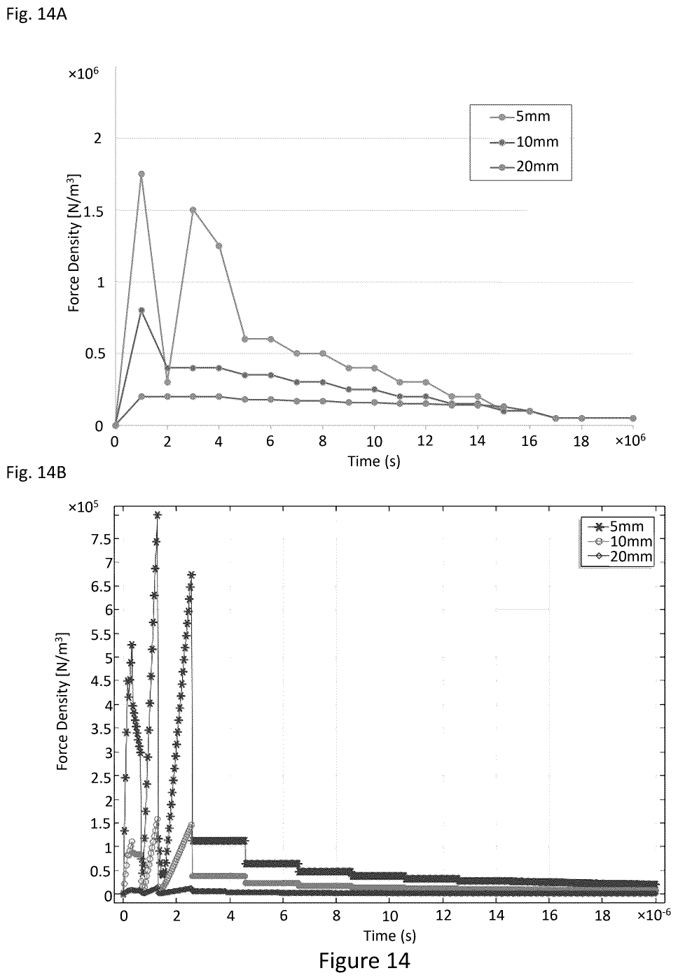

Data from the computer model and initial in vitro studies (without bacteria) confirmed the magnetic field components of the excitation field at the surface of the metal disks. However, the desired acoustic excitation from magnetic induction could not be reliably detected even with the transducers on the surface of the disk, which would not mimic future in vivo conditions. Despite several attempts to engineer an effective transducer, it was impossible to measure acoustic waves at the metal surface. In contrast, the simulated effect of the lift-off distance on the Lorentz force was in excellent agreement with the data obtained experimentally (FIG. 14A and FIG. 14B).

The transducer was then evaluated on disks with established biofilms and, regardless of the very weak ultrasonic waves, it produced desirable effects in terms of bacteria growth inhibition.

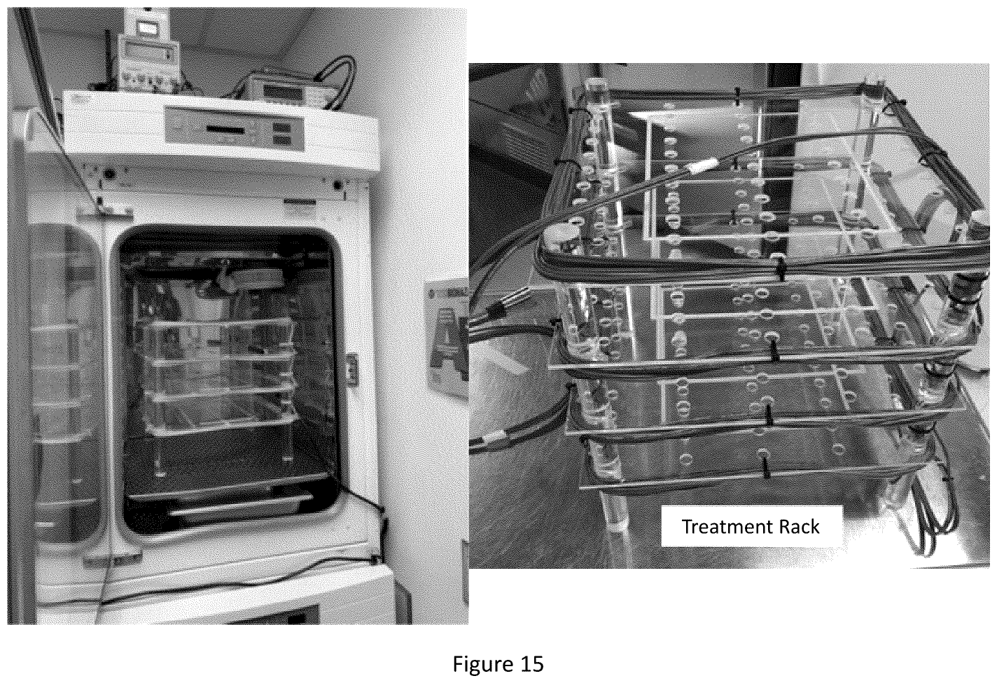

The data obtained guided in designing an experimental set-up to evaluate just the magnetic component of EMAT. It was reputed improbable that the very small ultrasound waves generated on the metal surface produced any significant effect on bacteria. To test this hypothesis, an EMF-only system was built consisting of an instrumented chain made of a simple coil obtained by wrapping an 18 AWG electrical wire around a polycarbonate rack designed to support well plates, a programmable function generator, a power supply, and a timer (FIG. 15). Two different systems were built to improve the throughput and increase efficiency.

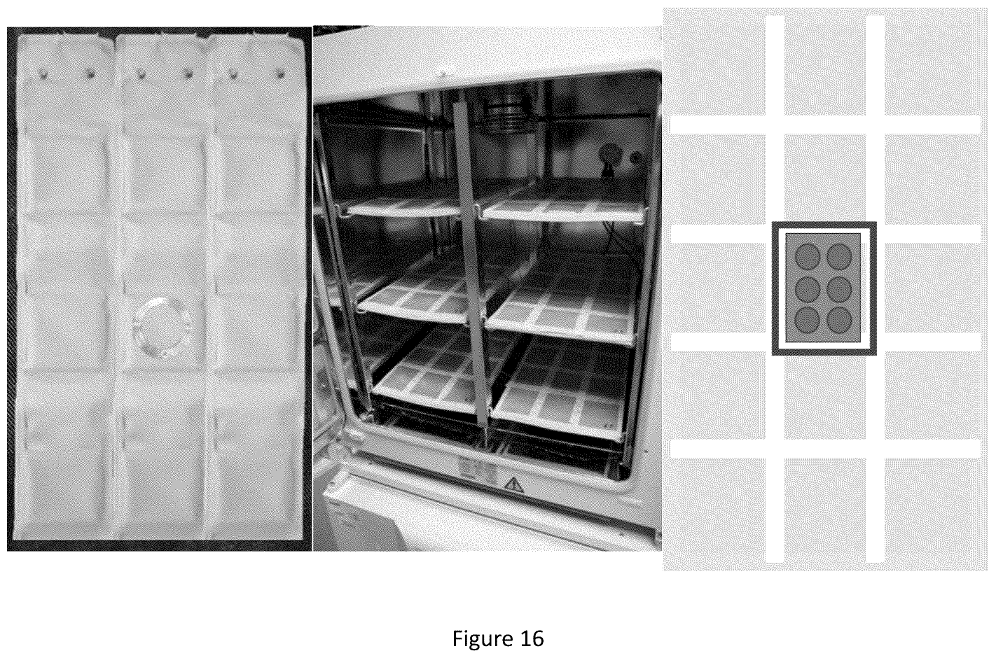

Simulation studies demonstrated that a system reduced to miniature coils driven by a small-factor board connected to a battery pack could deliver an equivalent EMF over a distance of several centimeters, which should adequately treat patients. Therefore, while testing various EMF conditions with the laboratory system, a set-up was engineered that could be adapted into a future wearable configuration. A coil-embedded mat was first engineered (FIG. 16, Left) to deliver the desired EMF to the tissue culture well plates containing the bacteria. The coil parameters were designed to replicate the local EMF value as close as possible and distributed in the mat at a distance that would deliver a uniform EMF over the mat area. The mat was engineered to be flexible and self-contained to avoid any possible contamination while in the incubator and be easily cleaned and disinfected.

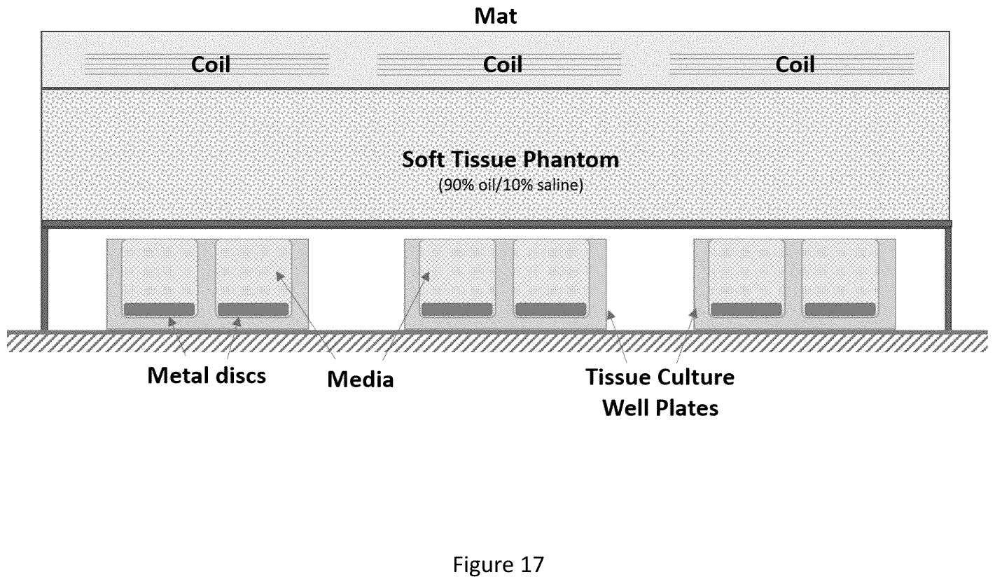

The mats were placed on top of the well plates (FIG. 17) at a distance of 30 mm which can be considered the average distance from the skin to possible metal implant location (normal BMI). Air, in this case, was the only media interposed between the mat and the well plate. Since the relative permeability of air and soft tissue is close, it was expected that this configuration represent the in vivo situation. To further evaluate if the dielectric properties would impact the EM pulse transmission, a soft tissue phantom was also constructed that could be interposed between the mat and the well plates (FIG. 17).

The phantom was made following a procedure described elsewhere. Briefly, an oil-in-gelatin dispersion was obtained by mixing 90% volume of aqueous gelatin and 10% volume of Canola oil. The aqueous gelatin was obtained by mixing 225 bloom gelatin (Sigma Aldrich) in 6% saline water. A surfactant (Triton X-100, Thermo Fisher) was also added as an emulsifier to decrease the surface tension between the oil and water. The oil mixture was contained in a sealed PVC bag that had an almost null dielectric loss factor. The dielectric properties of the soft tissue phantom were measured with an impedance probe over the frequency range relevant to the study. Measured dielectric properties were in agreement with well-accepted published results for human tissues. Since the conductivity of the material is mainly dependent on the saline concentration, while the permittivity is related to the oil volume percentage in the oil-in-gelatin dispersion, the phantom allowed mimicking different real-life condition, depending on the characteristics of the patient.

Staphylococcus epidermidis (S. epidermidis) ATCC 14990 was used for the biofilm experiments. The observed results were further confirmed by repeating said tests on S. epidermidis strain ATCC 35984. To test the above model the cells were first grown in Trypticase Soy Broth (TSB) (30 gl⁻¹ in purified water, autoclaved at 121° C. for 15 min; Becton, Dickinson and Company, Los Angeles, CA). The cells were incubated for 24 hours at 37° C. and then diluted 1:100 into TSB with glucose and plated. Five phases of experiments with this set-up have been successfully completed:

1) Evaluation of PEMFs ability to inhibit S. epidermidis biofilm formation on cell culture treated plastic plates.

2) Evaluation of PEMFs ability to eradicate fully formed S. epidermidis biofilms on cell culture treated plastic plates.

3) Evaluation of PEMFs ability to enhance the efficacy of antibiotic (specifically oxacillin and vancomycin) treatment against fully formed S. epidermidis biofilms on cell culture treated plastic plates.

4) Evaluation of PEMFs ability to eradicate fully formed S. epidermidis biofilms on titanium-alloy metal discs.

5) Evaluation of PEMFs ability to eradicate fully formed S. epidermidis biofilms when combined with magnetic functionalized nanoparticles (fNPs).

For the biofilm inhibition experiments PEMF was applied to all experimental plates immediately after the cells were plated, with the positive control being placed in a non-treated 37° C. incubator for the duration of treatment. Alternatively, for phases 2-4, cells were plated and then placed back in the non-treated 37° C. incubator for 24 hours (deemed adequate time for a biofilm to fully form through the literature and confirmed by preliminary experiments) before being exposed to PEMF.

In each phase, PEMF exposure was delivered for the following intervals:
0 hours (positive control group, in 37° C. incubator for entirety of experiment)
4 hours×2 cycles (exposed to PEMF hours 1-4, placed in 37° C. incubator hours 5-8, exposed to PEMF hours 9-12)
12 hours continuously
24 hours continuously

It is important to note that for all phase 3 trials, all PEMF exposure groups were tested as listed and compared to identical PEMF exposure times plus antibiotic. In phase 5 trials, PEMF exposure was delivered for alternate intervals of 0 hours, 3 hours, 6 hours, and 12 hours. This decision was due to the increased biofilm eradication ability of PEMF when combined with magnetic fNPs. As greater eradication was observed, it was deemed relevant to test shorter PEMF exposure intervals that are more applicable in the clinical setting.

At the conclusion of said experiments, crystal violet and alamarBlue assays were used to quantify the remaining viable biofilm in each well, and CFU/ml counts were used to quantify the planktonic (free-living) S. epidermidis cells.

### Phase 1 Results:

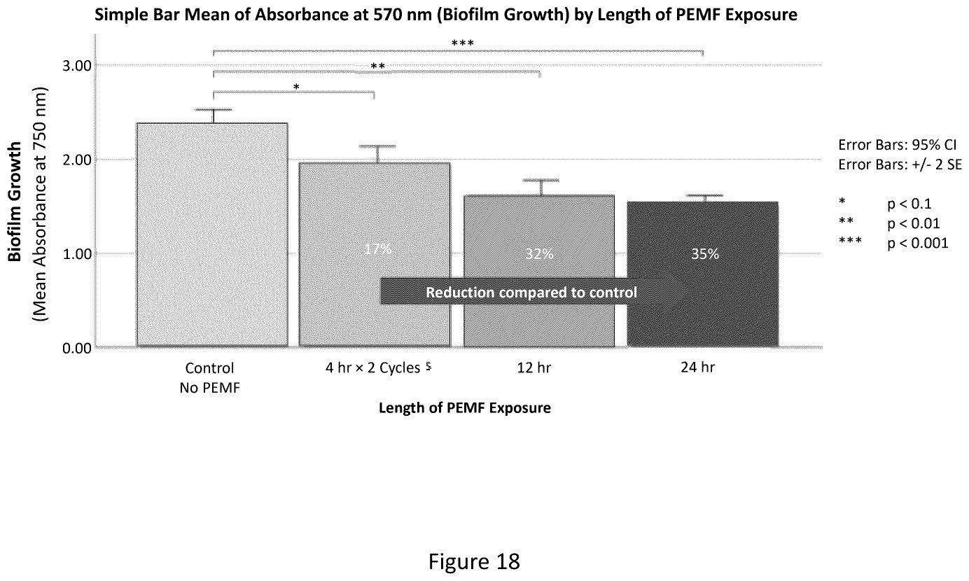

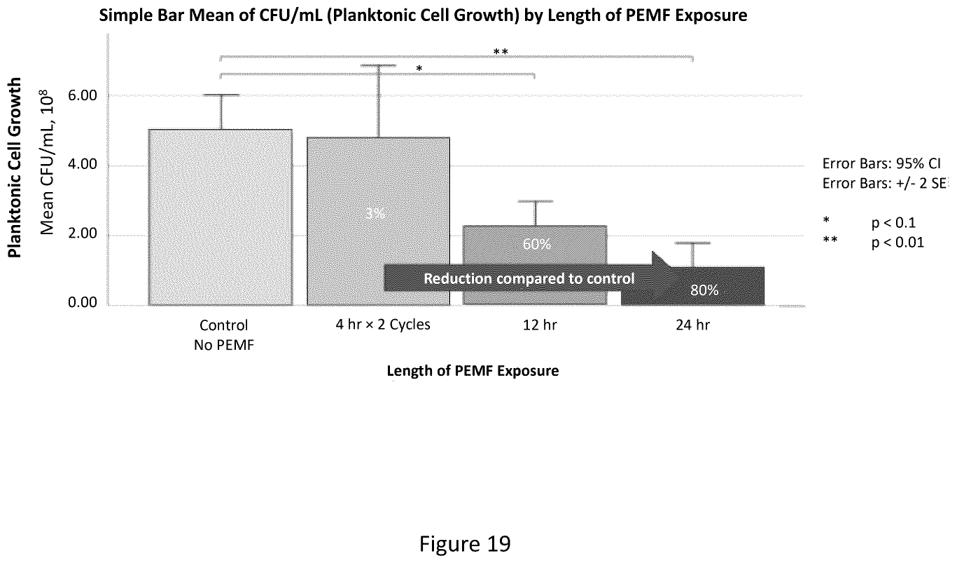

In the Phase 1 experiments described above it was shown that exposure to high-frequency PEMF (40 kHz) significantly inhibited S. epidermidis biofilm formation at all durations (FIG. 18), and significantly inhibited planktonic cell growth at 12 hours and 24 hours of exposure (FIG. 19). No significant inhibition in planktonic cell counts was seen at 4 hours×2 cycles of PEMF exposure.

### Phase 2 and Phase 3 Results:

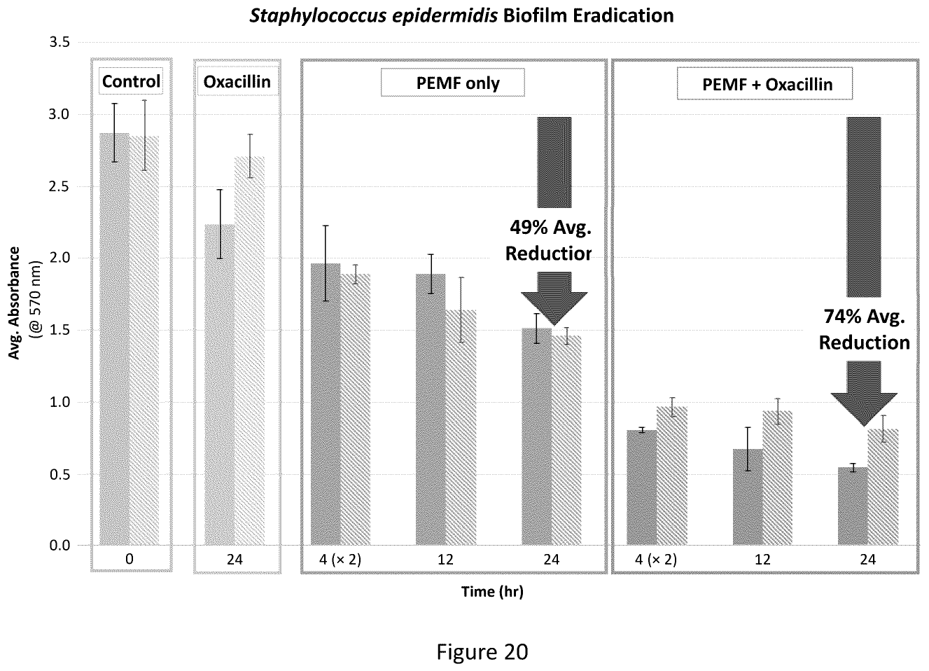

FIG. 20 shows PEMFs ability to eradicate pre-formed S. epidermidis biofilms in vitro. Furthermore, the combined treatment of PEMF exposure and antibiotics proved to be significantly synergistic as compared to the theoretical additive effect at all time points for both oxacillin and vancomycin. Neither oxacillin nor vancomycin alone significantly eradicated the S. epidermidis biofilm. This effect is far more relevant for an eventual clinical application, because for a patient presenting with an orthopedic or surgical site infection in the clinic, the bacteria will more than likely have already formed a full biofilm. Moreover, PEMFs ability to drastically improve the anti-biofilm efficacy of antibiotics will be paramount to future treatment because of antibiotics current inability to penetrate the extracellular polymeric matrix produced by the biofilm and therefore be ineffective in killing the Staph. cells.

The calculation performed to determine the theoretical additive effect of PEMF+antibiotic treatment and compare to the empirical effect of simultaneous PEMF and antibiotic treatment was a simple probability calculation: P(A)+(1−P(A))*P(B).

Further confirmation of results was obtained through SEM imaging of treated S. epidermidis biofilm samples, demonstrated in FIG. 4A through FIG. 4F. All samples were rinsed multiple times in phosphate buffered saline (PBS), glutaraldehyde fixed, ethanol dehydrated, dried, and sputter coated prior to imaging.

### Phase 4 Results:

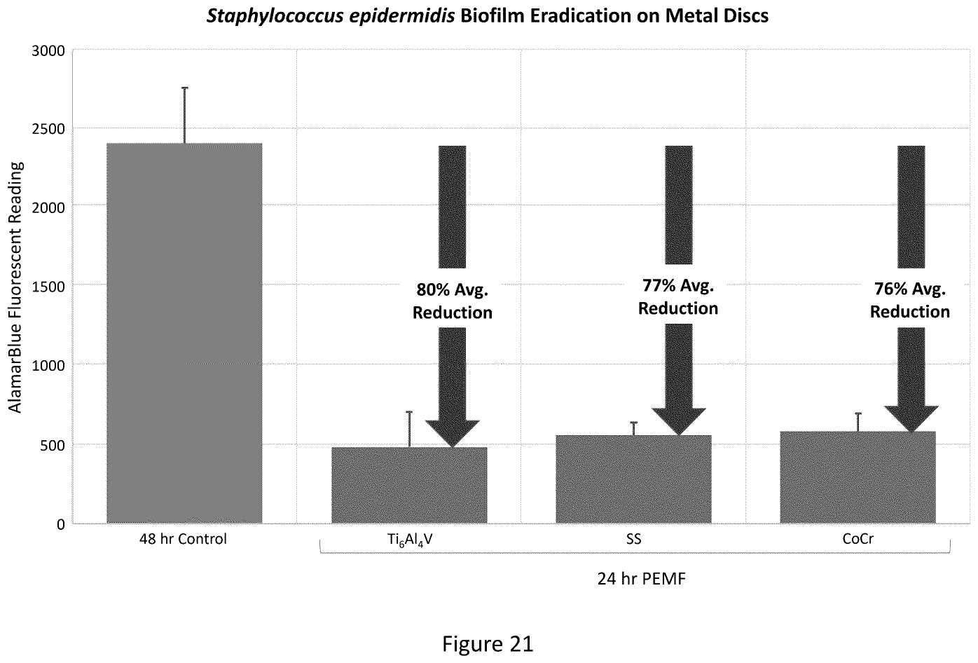

FIG. 21 shows even greater biofilm reduction by PEMF when the biofilm is grown on metal alloy discs. It was hypothesized that PEMF alone has a greater effect on the metal-alloy surface because of its ability to induce electrical charges on the metal, further disrupting the electrostatic forces working to keep the biofilm intact. Biofilm reduction by PEMF alone increased from 49% on a plastic surface (FIG. 18) to 80% on the titanium alloy surface (FIG. 21). Comparable reductions were observed on Stainless Steel (SS) and CoCrMo alloy discs. These results are very promising because they suggest PEMF can be a highly effective treatment biofilm-based orthopedic implant infection.

### Phase 5 Results:

Phase 5 of the project began initial testing of PEMFs ability to eradicate staphylococcal biofilms when combined empirically with magnetic functionalized nanoparticles (fNPs). It was hypothesized that the use of magnetic fNPs in combination with PEMF would be highly effective because fNPs can preferentially bind to bacteria or biofilm matrix components and then be oscillated by PEMF due to their magnetic nature, putting physical stress on the biofilm. Moreover, NPs have intrinsic bactericidal effects from their propensity to produce reactive oxygen species.

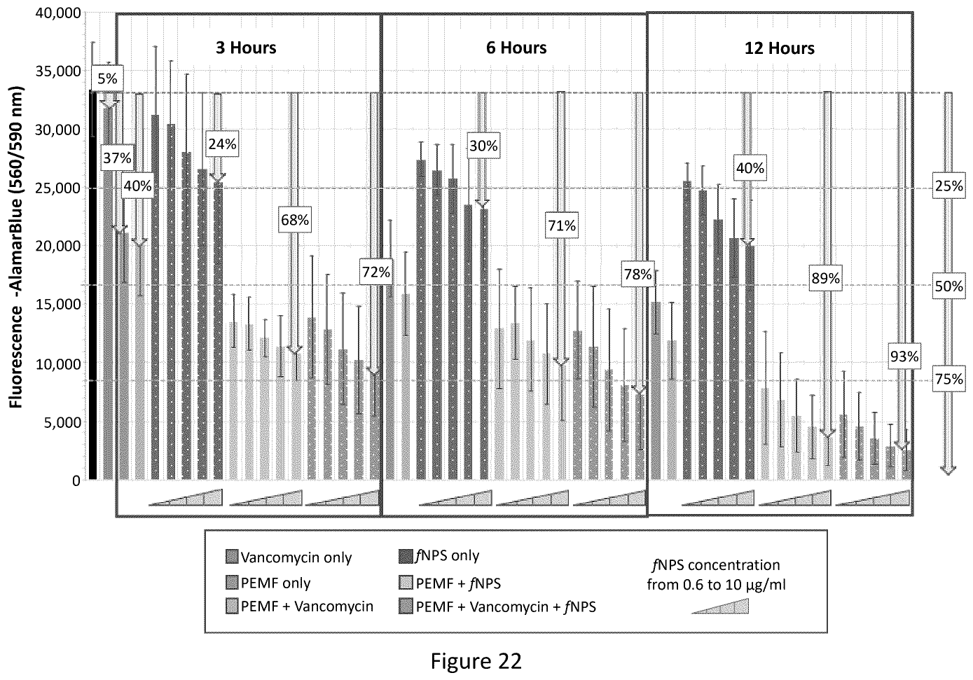

To perform this test, NPs functionalized with concanavalin A (Con-A) was used, due to Con-A's ability to preferentially bind to both the extracellular polymeric substance produced in Staphylococcal biofilms and S. epidermidis cells through a membrane receptor. FIG. 22 shows that drastic biofilm eradication was achieved through this technique. Moreover, the results showed that an increase in the eradication was achieved when PEMF/fNPs were combined to antibiotics. However, the increase although statistically significant was only marginal. A 93% eradication was observed at a fNPs concentrations of only 10 μg/ml when PEMF was combined with vancomycin.

Similar results were observed for S. aureus. However, replica of some of the tests on S. aureus is still undergoing. Tests involving the different metal alloys discs are also being finalized and initial data analysis shows an effect on Staph Aureus similar to that observed for Staph epidermidis.

### Example 5: The Use of Pulsed Electromagnetic Fields to Inhibit Staphylococcus epidermidis Biofilm and Planktonic Cell Growth

Staphylococcus epidermidis (S. epidermidis) has been implicated in multiple types of infection, including surgical site infections and orthopedic implant infections (Oliveira, W. F. et al., 2018, J Hop Infec. 98(2):111-117). Although these conditions were once successfully treated with antibiotics, the emergence of methicillin-resistant S. epidermidis strains (Namvar, A. E. et al., 2014, GMS Hyg Infect Control. 9(3):Doc 23) has created a need for a novel treatment. This issue is what the authors sought to address in the current study by determining the effects of a high-frequency pulsed electromagnetic field (PEMF) on S. epidermidis biofilm formation and planktonic cell growth. The primary hypothesis investigated was that S. epidermidis biofilm formation and planktonic cell growth is affected by the length of exposure to high-frequency PEMF.

The materials and methods employed in these experiments are now described.

S. epidermidis cells were grown through overnight incubation at 370 C in trypticase soy broth (TSB) media. On day two, cells were diluted 1:100 into TSB media with glucose and then aliquoted into 6-well cell culture plates for treatment. One plate was placed in a 370 C incubator where it remained for the entirety of the experiment (positive control group). Plates 2 through 4 were exposed to constant high-frequency PEMF in a separate 37° C. incubator. Specifically, Plate 2 was exposed to PEMF for 4 hours and then placed in the non-PEMF incubator for another 4 hours. It was then subjected again to PEMF exposure for another 4 hours, and returned to the non-PEMF incubator until the completion of the experiment at 24 hours (Plate 2 is referred to as 4-hour×2 cycles PEMF exposure group). Plate 3 was kept under PEMF exposure for 12 hours, then placed in the non-PEMF incubator for another 12 hours until completion of the 24-hour cycle (12-hour PEMF exposure group). Lastly, Plate 4 was placed in the PEMF incubator for the entirety of the experiment (24-hour PEMF exposure group). The PEMF apparatus was set to a high-frequency value of 40 kilohertz and a magnetic field amplitude of 1.2 mT for all groups and all replicates.

After the 24-hour cycle was completed for each plate, the level of planktonic cells was determined by serial dilutions and plating to measure the colony-forming units per milliliter (CFU/mL). Further, biofilm formation was assessed by staining biofilm-adhered cells with crystal violet (CV). After solubilizing the CV that stained the biofilm cells, the level of CV was measured via absorbance at a wavelength of 570 nanometers. The data from all replicate experiments were analyzed using a 1-way ANOVA test with Tukey post-hoc tests on SPSS.

The results of these experiments are now described.

High-frequency PEMF exposure significantly inhibited both S. epidermidis biofilm formation (p<0.001) and planktonic cell growth (p<0.001) as compared to the positive control group, supporting the primary hypothesis. More specifically, high-frequency PEMF exposure significantly inhibited biofilm growth in all groups (4-hour×2 cycle PEMF exposure group, p=0.004; 12-hour PEMF exposure group, p<0.001; 24-hour PEMF exposure group, p<0.001), and significantly inhibited planktonic cell growth in the 12-hour PEMF exposure group (p=0.029) and the 24-hour PEMF exposure group (p=0.001). n=9 for all groups tested for a total sample size of n=36 (FIG. 18 and FIG. 19).

High-frequency PEMF appears to be a viable solution to inhibit biofilm formation and planktonic cell growth of S. epidermidis. Further study, including animal and clinical trials, will be needed to derive a clinical treatment for S. epidermidis-based infections from this strategy. Still, these data confirm that high-frequency PEMF exposure is worthy of future study.

### Example 6: Establishment of a Model for Examining Musculoskeletal Infections

Musculoskeletal (MSK) infections are a major problem in orthopedic and trauma surgery with severe consequences for the affected patient. Such infections commonly arise from exposure of fractures to the battlefield environment at the time of injury. Both the direct exposure to pathogens in the field and the delivery of surgical services in low-resource settings put these patients at high risk of developing soft and deep tissue infections, some involving the joints, bones, and orthopedic implants. MSK infections are difficult to treat and are generally characterized by multiple surgeries, extended use of antibiotics, lengthy hospitalizations, and ambulatory care. Treatment costs of antibiotic-resistant infection have doubled since 2002, and now exceed $2 billion annually. Moreover, they are associated with high clinical failure rates and frequently result in loss of function and/or amputation.

Furthermore, the kinetics of treatment with conventional antibiotics favors the generation of antibiotic-resistant bacterial strains, which causes an immense physical and emotional burden, impacting readiness and often lasting a lifetime. Current strategies primarily concentrated on developing new antibiotics and novel antibiotic-delivery systems have had limited success and have not been able to avoid recurrent and persistent MSK infections. Moreover, even if an infection is eventually eradicated and the patient survives, a wounded soldier's bone may be too damaged by the time eradication occurs that chances of returning to the battlefield or even to active duty are low or nonexistent.

The difficulty in treating MSK infections raises in part by the capacity of bacteria to develop resistance mechanisms to evade the host immune system and antibiotic treatment. By forming a biofilm, bacteria become 100 to 1000-fold more tolerant to anti-microbial agents. Specifically, bacteria within biofilms are enclosed in a 3D network of a self-produced matrix of extracellular polymeric substances (EPS)—exopolysaccharides, proteins, extracellular DNA, and teichoic and lipoteichoic acids. EPS protects indwelling bacterial cells creating a physical barrier that limits antibiotics' ability to reach their target and inhibits phagocytosis by immune cells. Moreover, cells encased deep within the biofilm—known as persister cells—are downregulated metabolically into a slow-growing, essentially dormant state, decreasing the effectiveness of antibiotics designed to target active cell processes and making them less susceptible to cellular immune system detection. Furthermore, biofilm-based orthopedic implant infections are commonly caused by highly pathogenic gram-positive and gram-negative bacterium such as Staphylococci (aureus and epidermidis) and Pseudomonas aeruginosa (P. aeruginosa), respectively. Polymicrobial biofilms are also often found at orthopedic implant sites, highlighting the need for a broad-spectrum treatment.

With extremity trauma being the most common battlefield injury, post-traumatic osteomyelitis remains a significant concern for wounded soldiers and is associated with potential limb loss and substantial morbidity. The development of chronic osteomyelitis after incomplete initial debridement/treatment of either orthopedic implant-associated or post-traumatic bone infections remains a devastating and far too common outcome. Staphylococcus aureus (S. aureus) biofilm formation in the microtubule system of compact bones plays an important role in the pathogenesis of osteomyelitis, by much the same mechanism as in orthopedic implant infections. S. aureus infection of osteoblasts has been shown to significantly increase RANKL expression in their membrane. The increase in RANKL is likely to trigger osteoclast-induced bone resorption and bone destruction and may help explain why patients with osteomyelitis have significant bone loss. On top of this, a major factor in the high rates of recurrence and persistence—and a major challenge to treating osteomyelitis is the invasion of S. aureus into the host osteocyte lacunar-canalicular network.4 The 1.0-1.5 μm S. aureus cells invade this sub-μm network of canaliculi via self-mediated deformation and propagation into the network, a highly effective strategy by which it evades the host immune response. As canaliculi are too small for immune cells to enter, persisters can live safely within them for extended periods and cause a dangerous recurrent infection long after cessation of antibiotic treatment. Moreover, it has been demonstrated that bioburden— the ratio of bacteria to the number of available neutrophils, plays an important factor in establishing a biofilm infection. When the initial bacterial burden is large enough to overwhelm the immediate immune response and establish a mature biofilm, even a full immune response is not able to subsequently eradicate the infection. Consequently, recurrent and persistent infections now occur in more than 40% of wounded soldiers. Moreover, data from the Trauma Infectious Disease Outcomes Study (TIDOS) cohort have revealed a 27% rate of infectious complications in those evacuated after traumatic injury; this increases to 50% in the intensive care unit.

As a result of the previously successful results with treatment of biofilms with targeted nanoparticles and pulsed electromagnetic fields (PEMF), it was envisioned that these methods would provide successful methods of treatment for MSK infections. Additionally, PEMF has been previously shown to reinforce bone architecture, promote osteogenesis, and contrast the increase in RANKL and bone resorption associated with bacterial infection. As such, the targeted nanoparticle/PEMF method of treatment was envisioned to be even more successful in treatment of osteomyelitis.

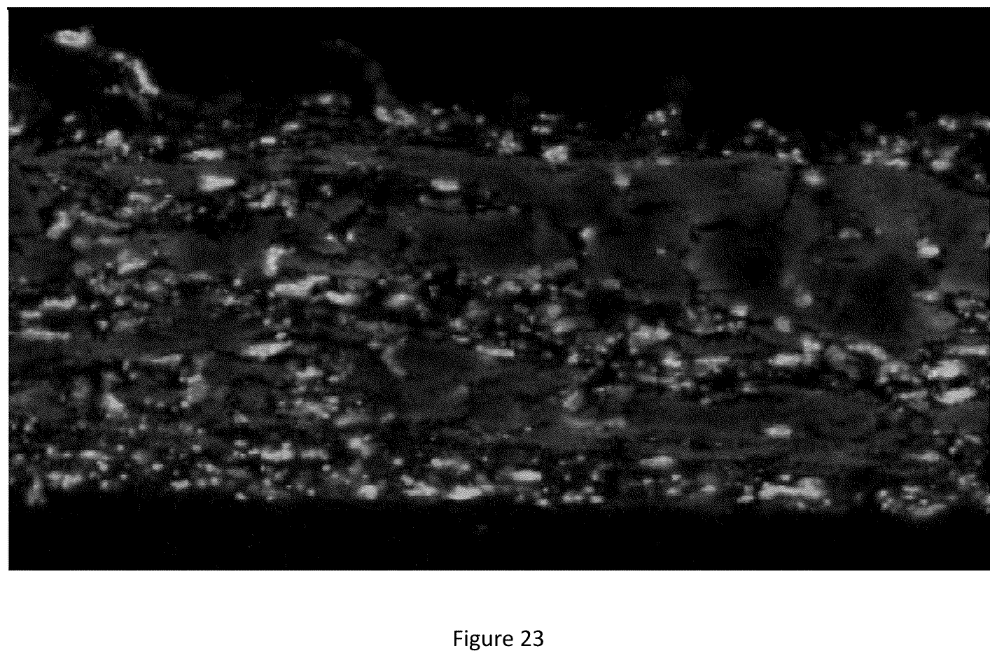

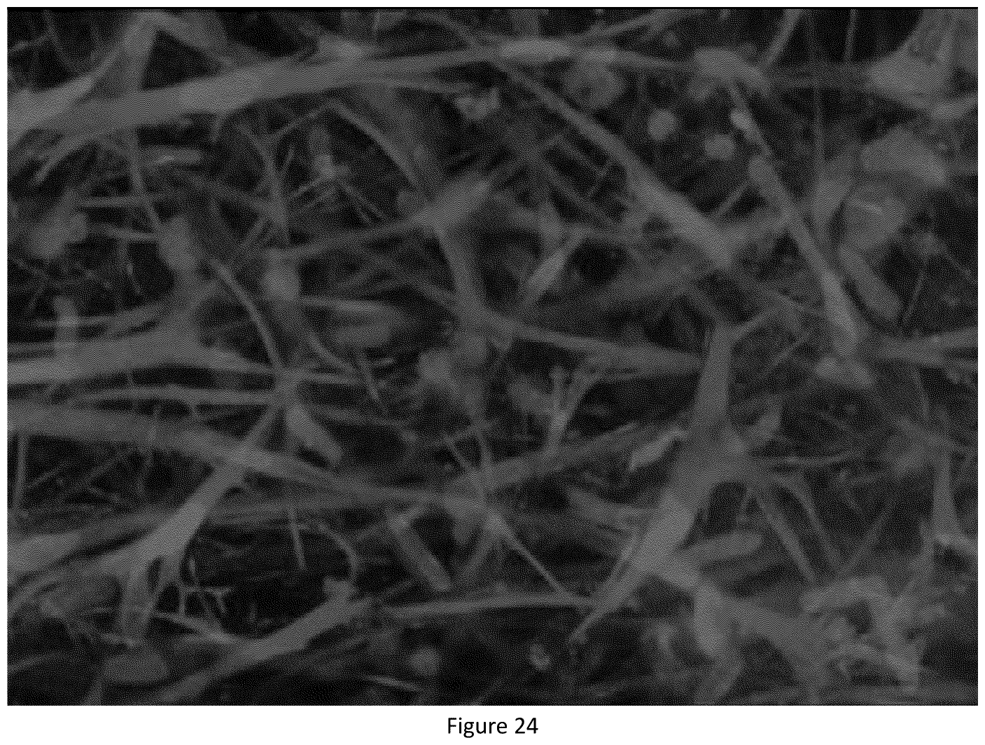

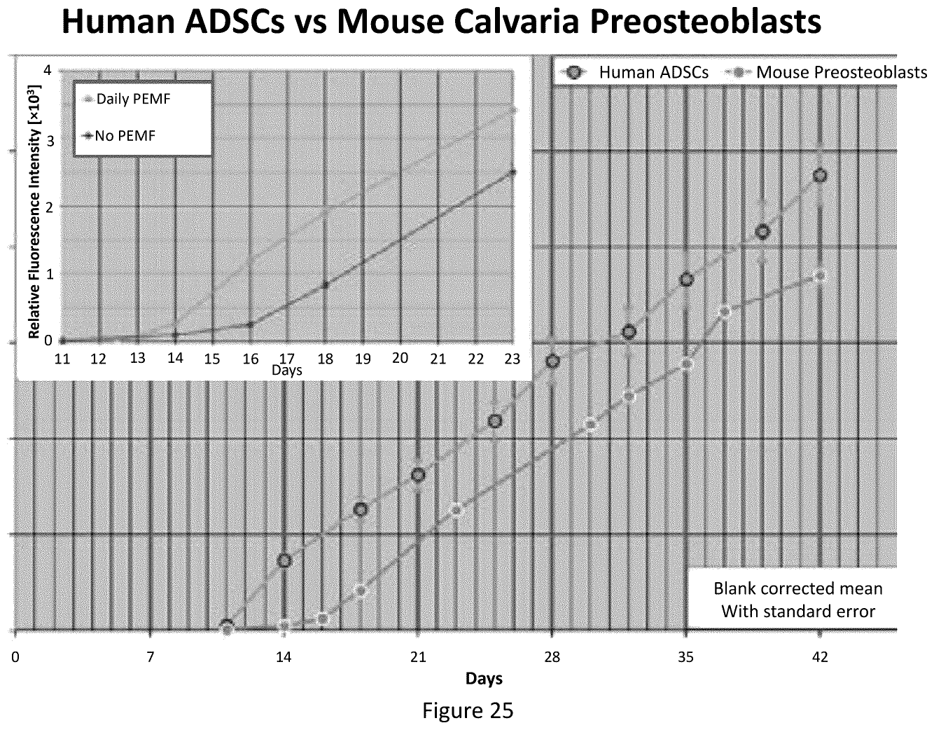

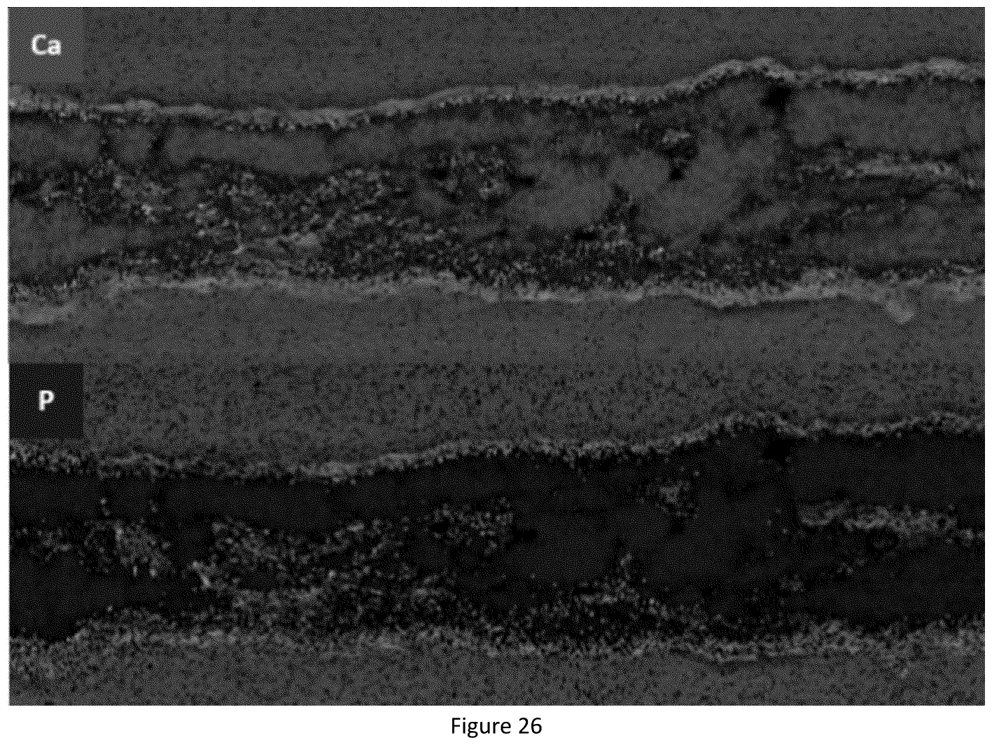

3D Bone Model: Osteocyte communication with osteoblasts and osteoclast precursors has been previously shown to be essential for normal bone turnover. As such, a 3D bone model was developed in order to directly evaluate biological stimuli for their capacity to induce or prevent osteolysis. Importantly, the model allows communication between osteocytes, osteoblasts, and osteoclast precursors. Moreover, the model provides an evaluation of the effects of several distinct stages of bone deposition using classical measures of bone quality for outcome determination, rather than short-term surrogates of osteoblast markers. Key elements of the model are the generation of two separate multilayers of mineralized bone matrix (FIG. 23) by polarized osteoblasts, differentiation and encasement of osteocytes exhibiting dendritic extensions in the mineralized matrix (FIG. 24), and the absence of a supporting scaffold. The model allows monitoring of important osteogenic markers such as mineral/matrix ratio, percent apatite, and mineral density, including quasi-real-time (FIG. 25) and terminal (FIG. 26) monitoring of mineralization.

Effect of PEMF on Bone Osteogenesis: The 3D bone model, with human-adipose-derived stem cells (hADSCs), was successfully utilized to determine the effects of timing on PEMF treatment on osteogenesis. Ring constructs were exposed to PEMF for 4 hours per day for the entire culture (Daily) or just during Day 1-Day 10, Day 11-Day 27, or Day 28-Day 63 and cultured without PEMF for the preceding or remaining days, and compared to no-PEMF controls. Osteogenesis was kinetically monitored by repeated fluorescence measurements of continuously present Alizarin Red S (ARS) and periodically confirmed by micro-CT. PEMF treatment induced statistically significant early-onset transient stimulation of the mineralization rate (˜4-fold, FIG. 25 inset). Therefore, in a defined, strong osteogenic environment, PEMF applied at different times was capable of further stimulating osteogenesis with the potential to enhance bone repair.

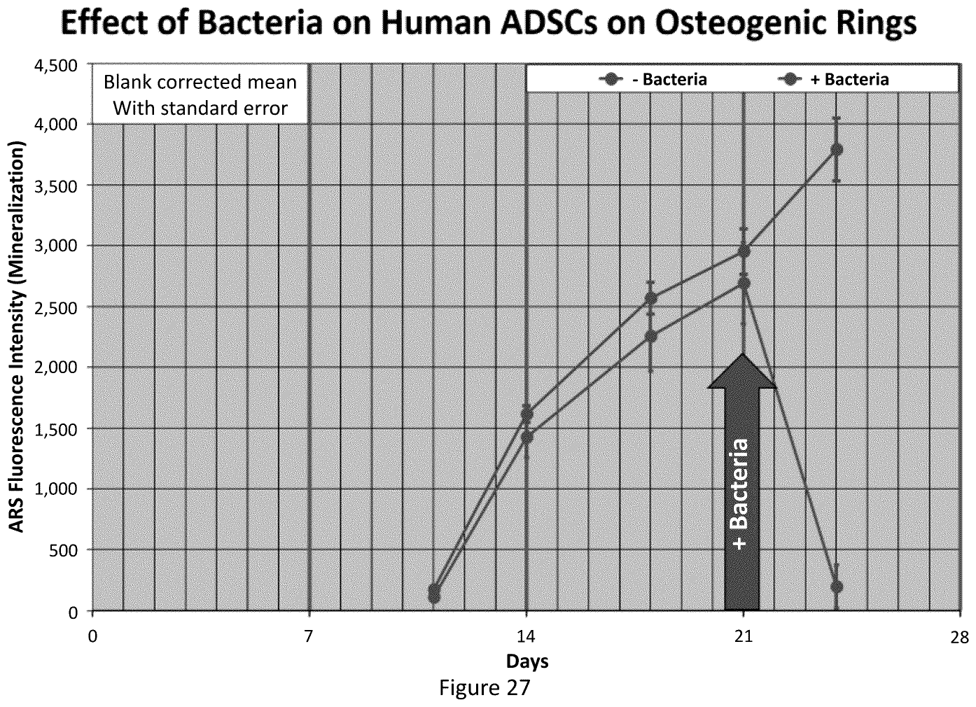

Bacterial Infection Reduces Preexisting Mineral Content: hADSCs were prepared in the established 3D bone model. When S. aureus was introduced to the hADSCs on osteogenic rings, a dramatic loss of ARS-fluorescence was observed within three days of infection. The reduction in fluorescence indicated a substantial loss of preexisting minerals and disruption/loss of the dendritic network (FIG. 27). Therefore, these results demonstrate that the 3D bone model with hADSCs is responsive to drivers of bone growth and differentiation as well as susceptible to the consequences of bacterial infection.

### Example 7: Treatment of Bacterial Infection of Bone by Targeted Nanoparticles and PEMF in a 3D Bone Model

Encouraged by successful development of a 3D bone model, the therapeutic ability of the previously prepared targeted nanoparticles and PEMF is examined in an infected 3D bone model.

Optimization of Antimicrobial Peptides: While Concanavalin-A was previously used for targeting nanoparticles to bacterial infections, additional antimicrobial peptides (AMPs) are investigated for optimal antimicrobial activity. In particular, LL-37 (SEQ ID NO:1) is investigated for its broad spectrum anti-microbial properties (i.e., Gram-positive and Gram-negative bacteria). Although free LL-37 requires relatively high concentrations to be effective, at which point it can stimulate a variety of responses in mammalian cells, these complications are addressed by immobilization of the peptide to nanoparticles, to increase local concentration, and modulation of the nanoparticle zeta potential to be positive, to reduce mammalian cell interactions.

Targeted nanoparticles will be prepared by reaction of nanoparticle-maleimide with antimicrobial peptides designed with an N-terminal cysteine. The coupling of the N-terminal cysteine and maleimide proceeds by a transyclization which protects the linkage from free thiols. As certain peptides, specifically LL-37, naturally form antiparallel dimers, tris(2-carboxyethyl)phosphine (TCEP) is added to the reaction to temporarily denature the peptides. After labelling of the nanoparticles, they are incubated in free peptide to form antiparallel tetramers, which is the functional conformation of the peptide for forming membrane-spanning channels. Subsequently, the nanoparticles are reacted with cysteine-lysine-fluorochrome-amides to fluorescently label the nanoparticles. Any remaining unreacted maleimide groups are quenched with a mixture of cysteine-amide and N-cysteine-lysine-amide, the ratio of which is controlled to manipulate the zeta potential of the nanoparticles.

Antimicrobial peptides being screened are:

N-terminal-cysteine-LL-37:
(SEQ ID NO: 1)
CLLGDFFRKSKEKIGKEFKRIVQRIKDFLRNLVPRTES
N-terminal-cysteine-WR12:
(SEQ ID NO: 2)
CRWWRWWRRWWRR
N-terminal-cysteine-WR18:
(SEQ ID NO: 3)
CWRRWWRRWWRWWRRWWRR
N-terminal-cysteine-WR34:
(SEQ ID NO: 4)
CRWWRWWRRWWRKWWRWWRRWWRKWWRWWRRWWRR

The WR12, WR18, and WR34 peptides are also tested with an N-terminal extension derived from LL-37, CLLGDF (SEQ ID NO:5) in place of a simple N-terminal cysteine (SEQ ID NOs:6, 7, and 8, respectively).

The WR peptides are synthetic peptides being investigated because WR12 was de novo designed to form standard amphipathic alpha-helices with 6 cationic charges and 50% hydrophobicity. Synthetic peptides provide significant advantages over naturally derived peptides, which tend to be toxic to host cells, easily degraded by proteases, susceptible to losing anti-microbial activity in the presence of physiological salt concentrations and costly to produce. WR12, in particular, has been demonstrated to have potent antibacterial activity against S. aureus (both methicillin- and vancomycin-resistant), S. epidermidis, and P. aeruginosa with significantly faster killing kinetics than traditional antibiotics, and is similarly potent against S. aureus persisters and established S. aureus, S. epidermidis, and P. aeruginosa biofilms. WR12 is thought to exhibit its anti-microbial effects through initial selective attraction to anionic lipids on the bacterial cell membrane, then permeabilization of the membrane, causing leakage of intracellular contents and cell death. One study showed it to cause greater than 95% leakage of preloaded intracellular calcein dye within 60 minutes of exposure to Staphylococcal membranes. Due to its direct effect on the cell membrane, WR12 is far more effective against bacteria in the dormant or quiescent state (persister and biofilm cell states, respectively) than traditional antibiotics, likely because it does not require bacterial metabolic activity to incite its killing mechanism, as antibiotics do to inhibit intracellular target processes. Moreover, the amphipathic nature and strong cationic charge of WR12 have been suggested to facilitate quicker diffusion through the biofilm EPS. The highly selective electrostatic attraction of WR12 to bacteria may also facilitate diffusion of WR12-functionalized MNPs into the canaliculi, allowing for more effective eradication of these immune-evasive persisters. Theoretically, drastically reducing the risk of recurrent infection. Finally, WR12 has been shown to have synergistic anti-microbial and anti-biofilm activity with other peptides and traditional antibiotics, and its effects are not inhibited by the presence of divalent cations. Imperatively, it also displays negligible cytotoxicity levels up to 20 times its minimum bactericidal concentration.

A key variable in making LL-37 more effective is its presentation to bacterial and mammalian cell membranes. The former to enhance killing and the latter to minimize contact and signaling as a side-effect of its antibiotic function. The work of Hassan et al. clearly demonstrated that high local density of immobilized Vancomycin (Vanc) arrayed on the surface of 200 nm MNP dramatically reduced the MIC (10-100-fold) relative to soluble Vanc. This is likely due to a fixed closer location between cell wall binding sites. The increased effectiveness was also capable of converting a Vanc-resistant strain to Vanc-sensitive. It was suggested that the benefits of immobilization and close proximity membrane interactions might be a generalizable paradigm for other conventional antibiotics.

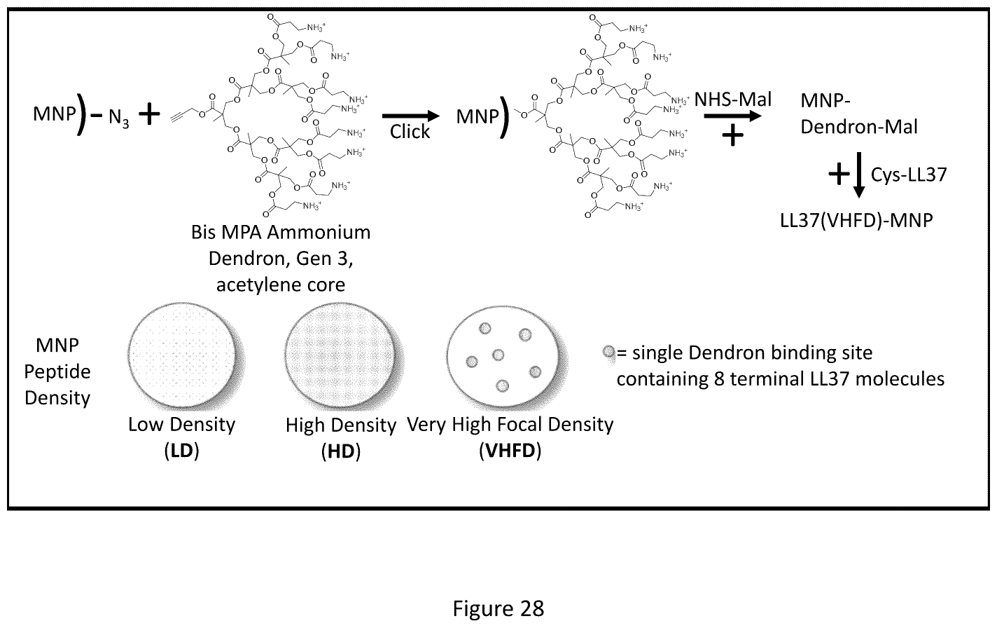

Consequently, the effectiveness of LL-37 and other AMPs are routinely evaluated at three different relative densities, Low Density (LD), High Density, and Very High Focal Density (VHFD). The first two are engineered by changing the peptide concentration and the coupling duration within an 8-hour interval. The latter is constructed based on the close proximity (very high density) of the end groups of a dendron and a very low number of conjugation sites (FIG. 28). In this way, a small number of peptides, but at a high-density, are used, thereby facilitating cooperativity in membrane disruption. By controlling the zeta potential, such nanoparticles do not closely approach mammalian cell membranes due to charge repulsion, thus offering specificity and safety.

Biofilm Metal Pin Assay for Bactericidal AMPs: For the purposes of mimicking the bacteria/metal interactions of implanted metal devices and prostheses, and providing a facile platform for screening selected AMPs, a modified Calgary peg 96-well assay is used. Suspended 1 cm sections (pins) of stateless steel and titanium Kirshner wire are used in place of the conventional plastic pegs. Both systems benefit from the fact that only biofilm-contained bacteria remain with the pin/peg to be assayed for bactericidal capacity of the treatment solutions. The removable pins are pre-treated in situ with 20% human platelet-poor plasma and cultured in medium containing the same to more closely approximate the in vivo environment and support biofilm formation by coagulates-positive SA. Following time-course or standard 2-3 day growth periods, the arrayed pins are washed and assayed for metabolic activity using AlamarBlue fluorescence with a bottom optic in a BMG plate reader. Subsequently, confirmation of bacterial killing is performed by pin removal to microfuge tubes, brief treatment with TrypLE (to disrupt fibrin-based biofilms), and low-power sonication before quantitation by conventional CFU assays.

Standardized screening assays are performed with 10 replicate pins/condition and the following variables evaluated: No treatment (Control), AMP alone, nanoparticles alone, AMP-nanoparticles alone, PEMF alone, PEMF+nanoparticles, PEMF+AMP-nanoparticles, and Vancomycin (0.5 ug/ml). Treatment of 4, 8, 18, 24, and 48 hours is examined. Pilot studies are performed using 2, 10 and 20 μg/ml of nanoparticles to establish a routine dosage range. Additionally, for treatments with high bacterial killing bacterial recovery is measured after treatment ends. The pin assay is well suited to this approach, allowing AlamarBlue assayed pins to continue in a treatment-free culture so that the few remaining bacteria have the opportunity to proliferate to more accurately quantifiable levels over the next 4, 12, or 24 hours and be re-assayed by AlamarBlue and CFU. This recovery period is paired with planktonic bacterial standards of 1, 10, and 100 bacteria per assay for comparison.

The above treatments are challenged by S. aureus Xen36 bioluminescent bacterial so that an additional marker of cell death is available, and experience is gained for the osteogenic ring assay (ring assay). In addition, it represents the most common infecting organism for PJI. Treatments that are strongly bactericidal are subsequently challenged with S. epidermidis and then a polymicrobial three-part infection with bioluminescent S. aureus (IVISbrite Staphylococcus aureus Xen36, Perkin Elmer), MRSA (IVISbrite™ Staphylococcus aureus Xen31), and P. aeruginosa (IVISbrite™ Pseudomonas aeruginosa Xen41) as individual bacteria or a mixture. In the latter case, the combination of three simultaneously immobilized AMPs (HNP-1, hBD-1, and LL-37) is tested, which has been shown to completely eradicate similar polymicrobial infections and demonstrated the advantage of utilizing peptide anchorage to increase bacterial membrane stress for killing.

Optimization of Nanoparticle Characteristics: Synthesized nanoparticles and targeted nanoparticles are characterized by biochemical assays for AMP content as described above and by SEM of wafer displayed particles as done for quantitative and morphological characterization of prosthetic wear particles in the range of 5-1000 nm. This generates targeted nanoparticle data on particle size, shape, and number. Experimentation begins with MNPs in the range of 60-120 nm in diameter (see above), and adjusted by increasing the core size during synthesis, when necessary. Biofilm penetration analysis uses MRSA (MW2)-GFP²⁰ and PA-GFP (ATCC 10145GFP) on biofilm pins with complementary Alexa Fluor fluorochrome labeled targeted nanoparticles (see synthesis) using confocal or 2-photon microscope (FIG. 24) (UCLA CNSI Core Facility). The strategy presented above for quenching the coupling reaction allows varying of fluorochrome color and content and adjustment of zeta potential with the introduction of primary amines, independent of, and without modifying, the coupled targeted nanoparticles. In addition, this generates MNPs that are PFA fixable, facilitating microscopic localization. Zeta potentials are determined by laser diffraction light scattering and tend to be +3 or greater to facilitate targeted nanoparticle ionic attraction to the negatively charged bacterial surface. Early data determine when high+zeta potential inhibits the bactericidal effects or leads to detrimental interactions with mammalian cells and loss of selectivity.

Identification of Optimal PEMF Signal: Initially, the above PEMF variables are screened using the above-described pin assay with S. aureus Xen36 and LL-37-MNP. The optimal conditions are used to screen other targeted nanoparticles and their presentation to bacteria. The best targeted nanoparticle(s)/presentations are then used to rescreen the characteristics of the PEMF signal. This is an essential process because it sets the stage for the approach to the in vivo animal model through the intermediate osteogenic ring model. Both models may reflect changes in optimal PEMF characteristics but benefit from the data collected from the higher throughput pin model. PEMF treatments used are characterized by a magnetic field amplitude from 0.5 to 2.0 mT and at least two different pulse frequencies (e.g., 5 kHz and 50 kHz), and a triangular waveform at different exposure times (e.g., 4, 12, and 24 hours) utilizing a custom PEMF device.

### Example 8: Examination of the Impact of Bacterial Infections on Mineralizing Live Bone and the Susceptibility to Killing by Targeted Nanoparticles/PEMF

With any given metal implant or device an infection confronts two interfaces, one with the metal surface as modulated by the in vivo environment, modeled with the pin assay, and the second with the surrounding bone, modeled with 3D osteogenic ring culture (ring culture). The bone interface is much more complex than the metal one, including osteoblast lineage cells and their derived osteocytes, robust collagen predominant extracellular matrix, and biologically deposited hydroxyapatite mineral and its accessory proteins. As indicated by clinical practice the bone interface is especially prone to bacterial colonization and the establishment of non-proliferative, metabolically inactive, persister bacteria that act a reservoir of bacteria capable of causing recurrence after antibiotic treatment has been terminated. Indeed, such cells protected from conventional antibiotics by biofilm and physical isolation from immune cells by sequestration in the osteocyte-derived lacuno-canalicular system are a major challenge in resolving bone infections. The most successful antimicrobial treatments from the pin assay are examined with the complex ring assay to determine efficacy in an environment approximating that in vivo. Ring culture is used with hADSC as it provides parallel osteogenesis to the more fully characterized mouse pre-osteoblast ring culture and is clinically relevant. In addition, it has been shown that it is responsive to bacterial growth with loss of the accumulated mineral (FIG. 27), a transition that allows greater opportunity of protected bacterial growth or dormancy.

Treatment of Bacteria in a 3D Bone Culture Model with Targeted Nanoparticles/PEMF: Osteogenic 3D ring cultures are established using hADSC expanded to fifth culture in monolayer using alpha-MEM supplemented with 2% human platelet lysate and passaged with TrypLE. Passaged cells are suspended in 6 mg/ml fibrinogen, mixed with calcium and thrombin, and cast in wells containing Teflon rings with tabs. After 1 hour at 37° C., rings are lifted with tabs and placed on supports in 24-well black-walled plates. Cells are cultured to day 11 in differentiation medium containing 10 nM estrogen, 10 nM ascorbate, 10 nM ascorbate-2-phosphate, 0.5 mM beta glycerol phosphate, 4 mM tranexamic acid, 100 ng/ml BMP9, 2% platelet lysate, and primosin as an antibiotic. On day 11 the media is replaced with fresh media with tranexamic acid removed, 10 nM dexamethasone and 0.5 ug/ml ARS added, and beta glycerol phosphate increased to 3 mM to support mineral deposition in mineralizing medium.

On Day 35, rings are inoculated for four hours with S. aureus Xen 36 or other bacteria, washed, cultured with feeding for two days to establish biofilm, and then subjected to treatment with the chosen targeted nanoparticle/PEMF antimicrobial treatment for between four hours and two days. The targeted nanoparticles used are fluorescently labeled during synthesis with the appropriate complementary Alexa Fluor dye so that nanoparticle penetration of the ring can be determined. In some instances, after treatment, culturing of rings is continued in treatment-free medium for up to 4 days to determine if, and to what extent, the infection recovers by monitoring bioluminescence, fluorescence, and CFU. In this case parallel cultures are established with planktonic bacteria at 1, 10, 100 cells without rings to determine the number of reservoir bacteria remaining after treatment and recovery. If no bacteria are detected after the recovery period of this treatment protocol, it is assumed that complete bacterial eradication has occurred.

Continuous culture of rings in ARS and periodic reading of fluorescence provide a stable record of hADSC differentiation down the osteoblast lineage to osteocytes marked by mineralization of the culture (FIGS. 25 and 27). Growth of bacteria is measured daily (6 rings per condition) by bioluminescence (Spectrum, Perkin Elmer) for bioluminescent S. aureus (IVISbrite; Perkin Elmer) and treatment is quantified by a decline relative to the pretreatment bioluminescence. Experiments are repeated with MRSA (MW2)-GFP20 or P. aeruginosa-GFP (ATCC 10145GFP) in order to determine their growth (BMG, Omega) and distribution by fluorescence microscopy. In both cases, ARS mineralization-dependent fluorescence is measured daily (FIG. 27). At the end of treatment, rings are fixed in 4% PFA at 4° C. overnight, demineralized, and the osteocyte dendrite network visualized by confocal and 2-photon microscopy (necessary to image the full depth of the construct at 700 microns) using Alexa Fluor phalloidin (FIG. 2; Thermo Fisher) of various colors and the membrane stain Dil (1,1′-dioctadecyl-3,3,3′3′-tetramethylindocarbocyanine perchlorate). Bacteria are imaged following staining with a fixation-insensitive GFP antibody. Dil is useful because it only stains osteocyte membranes in network-linked osteocytes and thus provides an early indicator of subsequent mineral loss and matrix/cellular degradation. These results enable determination of the extent of damage to the dendrite network and the degree of loss of the mineralized matrix under the absence and presence of bacteria, and the presence of a documented (pin assay) antimicrobial treatment with targeted nanoparticles/PEMF. Because of the complex bone-like environment, experiments are repeated with higher doses of nanoparticles/PEMF than were optimal in the pin assay or with different presentations (if applicable). Cell number are determined by counting Hoechst-stained nuclei by image analysis as a measure of proliferation or construct degradation.

Additional experiments are employed to determine if targeted nanoparticle/PEMF prevents formation of biofilm and ring degradation by applying nanoparticles/PEMF after the 4h inoculation and wash steps. Prevention of accumulated ARS staining loss during the following two usual bacterial growth days indicates biofilm prevention activity. Measuring bioluminescence, fluorescence and CFU from such a low inoculum provides a stringent test of whether it is actually possible to achieve bacterial eradication.

PEMF-Dependent Stimulation of Bone Repair During Treatment: The effects of PEMF alone and targeted nanoparticles/PEMF on recovery of bone-like character is examined in the ring model. Ring cultures infected with bioluminescent bacteria (6 rings/variable) are treated with nanoparticles/PEMF to the absence of bioluminescence and then transferred to mineralization medium with or without targeted nanoparticles and treated with or without PEMF for 7 days. ARS fluorescence and bioluminescence are measured daily. Nanoparticles are dosed every other day with medium change. Recovery is assessed in fixed intact rings and frozen sections by 2-photon and confocal microscopy using probes for osteocyte dendrites (phalloidin), osteocyte-derived E11 (early osteocyte marker), sclerostin (late osteocyte marker) (antibodies from R&D Systems), and Dil (Invitrogen) as a marker of dendritic network connectivity. Both mineralized and demineralized samples are evaluated, to take advantage of the ARS stain. Cell number is determined by counting nuclei by image analysis as a measure of proliferation.

Investigation of Toxicity on Human Cells: Osteogenic ring culture is predominantly used here to provide a bone-like environment for sequestration of bacteria to mimic their resistance to conventional antibiotics. However, it is also used in the absence of bacteria to determine whether optimized treatments from pin assay screens exhibit toxic effects on cells and processes relevant to bone repair. Conditions are tested for inhibition of proliferation of hADSC and osteoblasts using the AlamarBlue assay by exposing rings to 3 different doses of pin assay-selected targeted nanoparticle/PEMF treatment over 2 days, on day 1 and day 7, of ring culture. To test for osteocyte differentiation and function, ARS incorporation in rings is assayed between day 14 and day 21, a period of steady early ARS/mineral deposition. For treatments including PEMF, an arm of the study also includes a no-PEMF group, to control for PEMF only effects. In all cases, PEMF is 4h/day, daily and targeted nanoparticles are replenished every other day. Six rings are used for each condition.

### Example 8: Treatment of Bacterial Infections in a Mouse Prosthesis Model

After optimization of targeted nanoparticles and PEMF characteristics, the efficacy of the treatment is studied in vivo in a PJI animal model. The model offers the opportunity to study the evolution of the infection in quasi-real time. Moreover, the presence of the metal k-wire in the mouse joint and in contact with the bone not only reproduces the conditions encountered in arthroplasty but also mimics the interaction between bone, metal, and bacteria characteristic of any trauma fixation device.

To minimize the number of animals, a pilot study is first performed with the scope of verifying if the best treatment conditions previously identified are confirmed in mice. The best treatment identified is selected and compared to no treatment and standard-of-care (administration of antibiotics). For this pilot study 5 mice per condition are tested.

Preparation of S. aureus: A frozen stock of S. aureus Xen36 is streaked on TSB plates (Tryptic Soy Broth with 1.5% bacto agar, Teknova, Hollister, CA) containing 200 ug/mL kanamycin (Sigma-Aldrich, St Louis, MO) to ensure the stability of the lux operon and incubated overnight at 37° C. Single colonies exhibiting bioluminescence are selected and cultured overnight for 16 hrs in TSB+200 μg/mL kanamycin in a shaking incubator at 196 rpm. Subcultures (2 hr; 1:50 dilution) yielding mid-logarithmic phase cells are pelleted via centrifugation, resuspended and washed in PBS, and quantified by spectroscopy (600 nm; BioMate 3, ThermoFisher Scientific).

Murine model of periprosthetic joint infection (PJI): A murine model is used to assess the efficacy of treatments. Briefly, twelve-week-old, 20-25 g C57BL/6 wild-type mice (Jackson Laboratory, Bar Harbor, ME) are used for all experiments. In some experiments, 12-week-old LysEGFP mice, a genetically engineered mouse line that possesses green-fluorescent myeloid cells (mostly neutrophils) due to a knock-in of enhanced green fluorescence protein (EGFP) into the lysozyme M gene, are used. All mice are housed 4 per cage and stored with an appropriate 12-hour light/dark cycle with free access to water and a standard pellet diet. Mice are anesthetized via inhalation of isoflurane (2%). A skin incision is made over the right knee, followed by a medial parapatellar arthrotomy to expose the distal femur. The femoral medullary canal is broached and sequentially reamed using a 25-gauge followed by a 21-gauge needle. A medical-grade titanium Kirschner wire (0.8 mm in diameter, 6 mm in length; DePuy Synthes, Warsaw, IN) is then placed in a retrograde fashion into the femoral medullary canal and left proud with 1 mm of wire protruding into the joint space. The wire tip is inoculated via pipette with 1×10³ CFUs of S. aureus Xen36 in 2 μL of PBS. Sterile control mice receive 2 μL of sterile saline (0.9% NaCl) following an identical surgical process. The surgical site is then closed using polyglycolic acid 5-0 sutures. To ensure proper placement of the implant, mice undergo high resolution x-ray imaging on PODO using an IVIS Lumina X5 (PerkinElmer, Waltham, MA).

Quantification of in vivo S. aureus burden (in vivo bioluminescence imaging): Mice are anesthetized with inhalation isoflurane (2%) and in vivo bioluminescence imaging (IVIS) is performed using the Lumina II® imaging system (Caliper Life Sciences) as previously described.

Quantification of neutrophil recruitment to the infected post-operative joint (in vivo fluorescence imaging): LysEGFP mice, which possess fluorescent neutrophils, are anesthetized with inhalation isoflurane (2%) and in vivo fluorescence imaging is performed using the Lumina II® imaging system (Caliper Life Sciences) as previously described. EGFP-expressing neutrophils at the post-operative site are visualized using the GFP filter for excitation (445-490 nm) and emission (515-575 nm) at an exposure time of 0.5 seconds.

Visualization of biofilms: Mice are euthanized, implants are harvested, and biofilm formation on the intra-articular end of the implants is visualized using a field emission variable-pressure scanning electron microscope (VP-SEM) (FE-SEM Zeiss Supra VP40). VP-SEM enables the direct visualization of biofilms without the need for sputter-coating.

Quantification of adherent S. aureus bacteria on the implants: Bacteria are detached from the implants by sonication in 1 ml 0.3% Tween-80 in TSB for 10 minutes followed by vortexing for 5 minutes. Adherent CFUs are counted after overnight culture.

Biodistribution of targeted nanoparticles and Toxicological Responses: The short-term (acute) toxicity of targeted nanoparticles is investigated after administration by intra-articular injection. The mice are sacrificed 24 h after injection, and blood samples are collected in heparin-coated tubes and centrifuged for 10 minutes at 1000×g within 30 minutes of collection to obtain the plasma sample. Blood chemistry, liver, and kidney functional parameters, total leukocyte count (WBC), erythrocyte count (RBC), platelets (Plt), hemoglobin (Hgb), and hematocrit (Hct) are detected. IL-6, IL-1β, and TNF-α levels in plasma are analyzed using ELISA kits according to the manufacturer's instructions. Lipid peroxidation is assessed by measuring the malondialdehyde (MDA) levels.

Histological analysis of the major organs is performed to see if targeted nanoparticles could induce pathological changes. After the blood is collected, the mice are killed by cervical dislocation. The organs are isolated by incision immediately after necropsy, fixed in a 10% formalin solution containing neutral phosphate-buffered saline (PBS), and stored at 4° C. The tissues are routinely processed, embedded in paraffin, sectioned at 3-5 mm, and then stained with hematoxylin and eosin (H&E) for microscopic examination.

Although the targeted nanoparticles are injected locally, they may translocate to other parts of the body after gaining access to the systemic circulation. The biodistribution of targeted nanoparticles assessment is based on the quantification of iron in excised tissue samples using an inductively coupled plasma-mass spectrometry (ICP-MS) method. The major organ samples are weighed, digested, and analyzed for iron content. Briefly, before the elemental analysis, the tissue samples of interest are homogenized for 60 s with a tissue homogenizer (Fisher Scientific). All the homogenates are transferred to Teflon containers and acidified with 5 mL of 100% ultrahigh purity nitric acid and digested at 95° C. for 3 hr before drying and re-dissolving in 5% nitric acid.

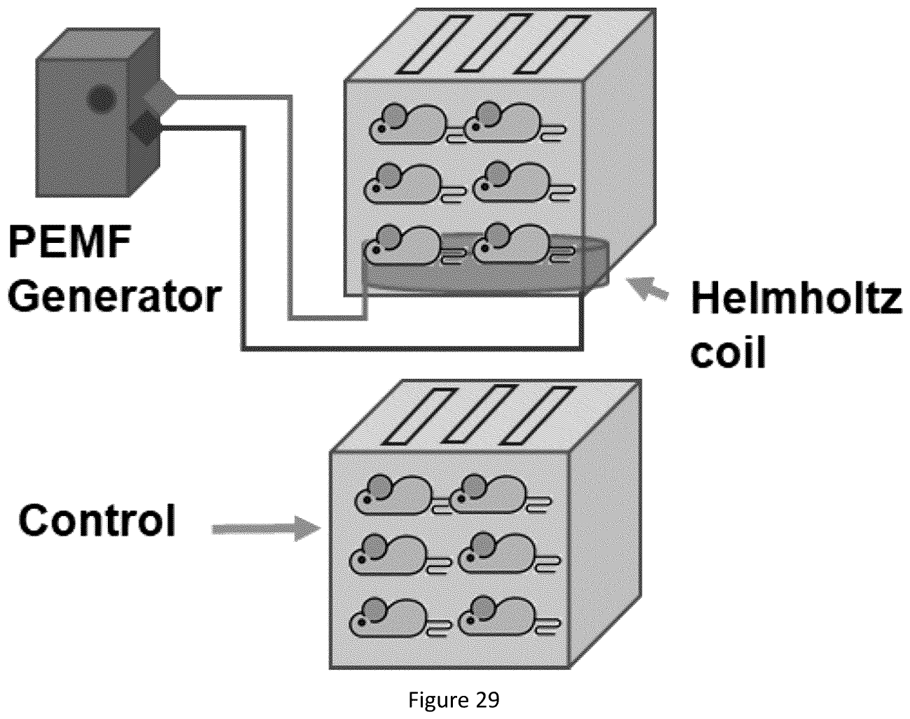

PEMF Apparatus: An apparatus adapted to accommodate mouse cages will be utilized. Briefly, an AC power supply and AC/DC converter provide 48 V DC power through a switching mode power supply (SMPS), and energy in the form of pulses is transferred to the PEMF-generating coil. The pulse output is 28 Hz, and a driving pulse in the form of a triangular wave is generated using a microprocessor (ATtiny2313A, Microchip, Chandler, AZ, USA) with a duty ratio of 50%, which is the on-time to off-time ratio. A 3V driving pulse is generated, and current amplification for driving the coil is obtained via a solid-state relay (SSR). The SSR plays a key role in transferring energy to the magnetic field coil by amplifying the 3 V pulse signal generated by the microprocessor to 48 V. The coil is placed under the cage (FIG. 29).

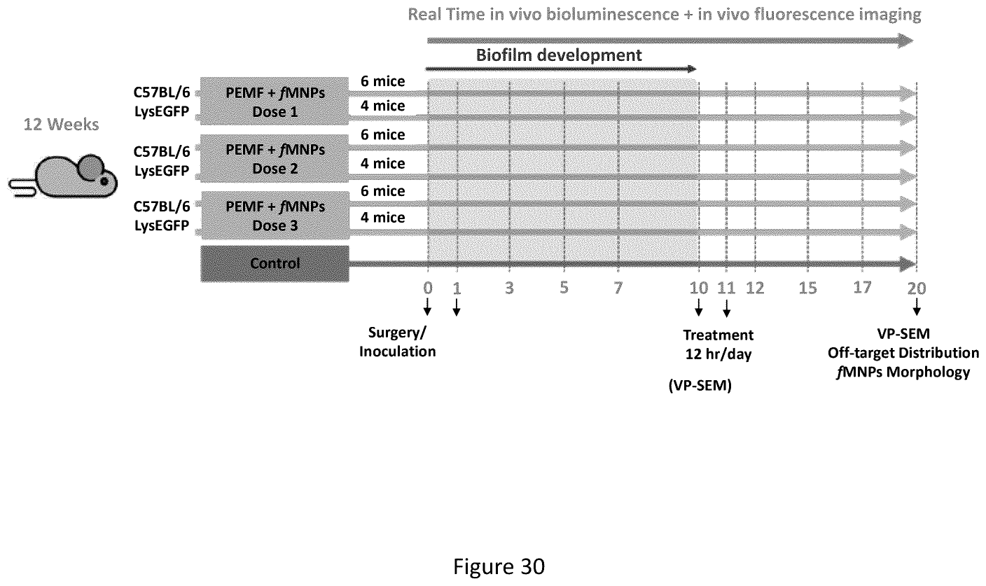

Pilot study: An outline of the pilot study is shown in FIG. 30. The rationale behind the pilot study is to determine the optimal dose of the targeted nanoparticles and to verify that the in vitro treatments actually translate to the animal study.

Four groups of mice are tested. One group serves as control (no treatment), the other three groups receive the optimal treatment from the in vitro studies with targeted nanoparticles at three different dosages. The two different types of mice are used in each group to allow both in vivo bioluminescence and in vivo neutrophil response quantification. PEMF signal characteristics are derived from the in vitro results. After surgery and inoculation, real time bioluminescent and fluorescence imaging is carried out at 0, 1, 3, 5, 7, and 10 days. Full biofilm formation is confirmed at 10-days post-surgery via in vivo imaging. At day 10, local administration of targeted nanoparticles are delivered at the joint site by injection according to a dosage established in vitro.

The treatment groups then undergo PEMF treatment for 12 hour a day for two consecutive days (days 10 and 11). Real time bioluminescent and fluorescence imaging continues at days 11, 12, 15, 17, and 20 post surgery. At 20-days post-surgery mice are sacrificed. Eradication of biofilm from the k-wire is confirmed via VP-SEM. Off target distribution to target organs is assessed according to reported procedure. Isolation of targeted nanoparticles from tissues is achieved using a protocol developed to isolate nanoparticles from orthopedic implants and tissues. Briefly, 100 mg of tissue from the joint site and various organs undergoes enzymatic digestion with proteinase K followed by a density gradient ultracentrifugation to purify the nanoparticles. Isolation, collection, and display of the nanoparticles occur via ultracentrifugation on Si-wafer coated with a monolayer of marine mussel glue. Analysis and characterization of the particles is performed via SEM (Zeiss Supra VP-40) and EDX analysis.

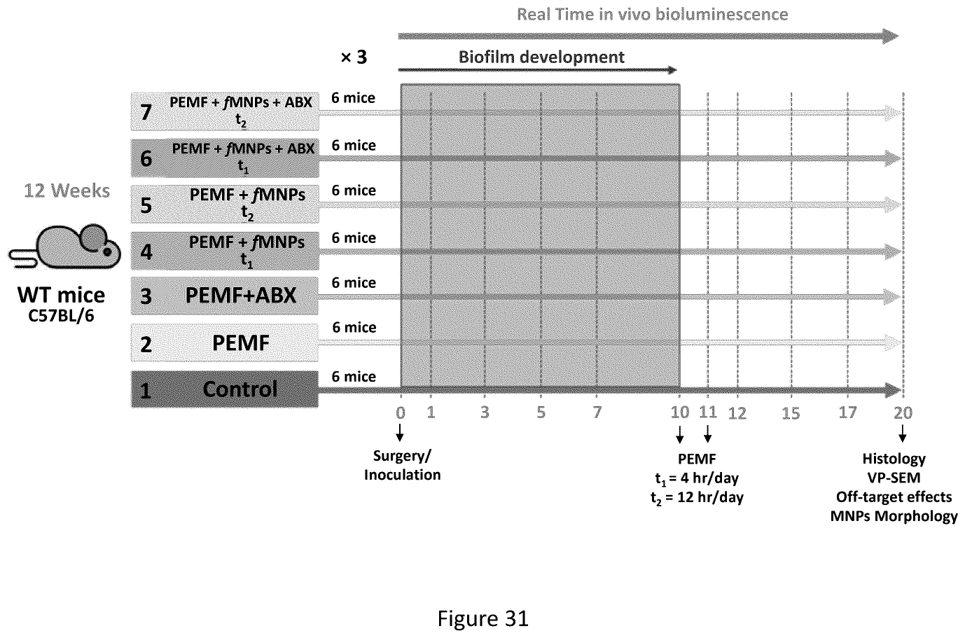

Main Study: An outline of the main study is shown in FIG. 31. Forty-two mice are divided into seven groups. In the first group, mice undergo surgery but do not receive any treatment (control). In the other groups mice receive treatment on days 10 and 11 after surgery. In group 2, only PEMF treatment is administered for 12 hr/day. In group 3, treatments with PEMF and vancomycin (120 mg/kg twice daily) are carried out on two days for 12 hr/day. In groups 4 and 5 PEMF and targeted nanoparticles (PEMF characteristics and nanoparticle dosage derived from pilot study) are administered at 12 hr/day in group 4 and 4 hr/day in group 5. Finally, groups 6 and 7 are repeated treatments of groups 4 and 5 with the addition of vancomycin. Each experiment is run in triplicate. Real time in vivo bioluminescence is carried out on the highlighted intervals in FIG. 31. Histology, quantification of bacteria on the implant, implant surface analysis via VP-SEM, nanoparticle content in tissue, morphological analysis of nanoparticles in tissue, and off-target effects are examined after euthanasia on day 20 after surgery.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for disrupting a biofilm comprising:
a) contacting a biofilm with a nanoparticle, wherein the nanoparticle comprises:
one or more metals; and
one or more targeting ligands on the surface of the nanoparticle;
b) interacting the one or more targeting ligands with one or more selected from the group consisting of: the biofilm matrix, a cell membrane of a bacterium in the biofilm, and a cell wall of a bacterium in the biofilm;
c) activating the nanoparticles by exposing them to an electromagnetic field; and
d) disrupting the biofilm via the activated nanoparticles.

2. The method of claim 1, wherein the one or more metals are one or more selected from the group consisting of: gold, silver, titanium, iron, zinc, and oxides thereof.

3. The method of claim 1, wherein the one or more targeting ligand is selected from the group consisting of a lectin, a quorum sensing (QS) signaling molecule, and combinations thereof.

4. The method of claim 1, wherein the one or more targeting ligands are one or more selected from the group consisting of: concanavalin-A, LL-37 (SEQ ID NO:1), WR12 (SEQ ID NO:2), WR18 (SEQ ID NO:3), WR34 (SEQ ID NO:4), LL-37-Linker-WR12 (SEQ ID NO:6), LL-37-Linker-WR18 (SEQ ID NO:7), and LL-Linker-WR34 (SEQ ID NO:8).

5. The method of claim 1, wherein the biofilm is located within a subject.

6. The method of claim 5, wherein the method further comprises administering an effective dose of at least one first therapeutic agent to the subject.

7. The method of claim 6, wherein the at least one first therapeutic agent is one or more selected from the group consisting of an antibiotic, an antiseptic, and combinations thereof.

8. The method of claim 7, wherein the antibiotic is selected from the group consisting of: oxacillin, vancomycin, cephalexin, gentamicin, augmentin, amikacin, and combinations thereof.

9. The method of claim 6, wherein the at least one first therapeutic agent is administered to the subject by at least one method selected from the group consisting of: intra-respiratory, nasal, topical, oral, intravenous, intraperitoneal, intramuscular, transmucosal, buccal, rectal, vaginal, sublingual topically, and combinations thereof.

10. The method of claim 6, wherein the at least one nanoparticle comprises at least one second therapeutic agent.

11. The method of claim 10, wherein the at least one second therapeutic agent is selected from the group consisting of: anti-viral agents, anti-bacterial agents, anti-biofilm agents, chemotherapeutic agents, anti-inflammatory agents, antiseptics, anesthetics, analgesics, pharmaceutical agents, small molecules, peptides, nucleic acids, and combinations thereof.

12. The method of claim 5, wherein the composition is administered to the subject by at least one method selected from the group consisting of: intra-respiratory, nasal, topical, oral, intravenous, intraperitoneal, intramuscular, transmucosal, buccal, rectal, vaginal, sublingual topically, and combinations thereof.

13. The method of claim 1, wherein the electromagnetic field is one selected from the group consisting of: a static electromagnetic field, a time-varying electromagnetic field, and a pulsed electromagnetic field, and a combinations thereof.

14. The method of claim 13, wherein the pulsed electromagnetic filed has a pulse length ranging between 0.01 seconds to 24 hours.

15. The method of claim 1, wherein the electromagnetic field has a frequency ranging between 1 to 800 kHz.

16. The method of claim 1, wherein the electromagnetic field has an amplitude ranging between 0.5 to 2 mT.

17. The method of claim 1, wherein the electromagnetic field has a waveform selected from the group consisting of: a square waveform, a triangular waveform, and combinations thereof.

18-19. (canceled)

20. The method of claim 6, wherein the administration of the at least one first therapeutic agent occurs at least partially simultaneously with at least one of: administration of the nanoparticles and positioning in electromagnetic field.

21. The method of claim 6, wherein the administration of the at least one first therapeutic agent occurs before administration of the nanoparticles and positioning in electromagnetic field.

22. The method of claim 6, wherein the administration of the at least one first therapeutic agent occurs after administration of the nanoparticles and positioning in electromagnetic field.
